SEQUENCE LISTING (208 sequences)

SEQ ID NO: 1 - protein, 247 aa, Artificial sequence
  Synthesized polypeptide
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 2 - protein, 29 aa, Artificial sequence
  Synthesized polypeptide
AAIEVMYPPPYLGGGGSGGGGSGGGGSLE

SEQ ID NO: 3 - protein, 185 aa, Artificial sequence
  Synthesized polypeptide
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 4 - DNA, 741 bp, Artificial sequence
  Synthesized polynucleotide
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgg

SEQ ID NO: 5 - DNA, 90 bp, Artificial sequence
  Synthesized polynucleotide
gcggccgcaattgaagttatgtatcctcctccttacctaggtggcggcggttctggtggcggcggttctggtggcggcggttctctcgag

SEQ ID NO: 6 - DNA, 555 bp, Artificial sequence
  Synthesized polynucleotide
gatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatc

SEQ ID NO: 7 - DNA, 1386 bp, Artificial sequence
  Synthesized polynucleotide
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgggcggccgcaattgaagttatgtatcctcctccttacctaggtggcggcggttctggtggcggcggttctggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatc

SEQ ID NO: 8 - protein, 160 aa, Artificial sequence
  Synthesized polypeptide
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 9 - DNA, 480 bp, Artificial sequence
  Synthesized polynucleotide
cagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggacccacgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 10 - DNA, 1311 bp, Artificial sequence
  Synthesized polynucleotide
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgggcggccgcaattgaagttatgtatcctcctccttacctaggtggcggcggttctggtggcggcggttctggtggcggcggttctctcgagcagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggacccacgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 11 - protein, 20 aa, Artificial sequence
  Synthesized polypeptide
AAAGGGGSGGGGSGGGGSLE

SEQ ID NO: 12 - DNA, 60 bp, Artificial sequence
  Synthesized polynucleotide
gcggccgcaggtggcggcggttctggtggcggcggttctggtggcggcggttctctcgag

SEQ ID NO: 13 - DNA, 1356 bp, Artificial sequence
  Synthesized polynucleotide
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgggcggccgcaggtggcggcggttctggtggcggcggttctggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatc

SEQ ID NO: 14 - DNA, 1281 bp, Artificial sequence
  Synthesized polynucleotide
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgggcggccgcaggtggcggcggttctggtggcggcggttctggtggcggcggttctctcgagcagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggacccacgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 15 - protein, 6 aa, Artificial sequence
  Synthesized polypeptide
SGYNWH

SEQ ID NO: 16 - protein, 16 aa, Artificial sequence
  Synthesized polypeptide
YIHYTGSTNYNPALRS

SEQ ID NO: 17 - protein, 9 aa, Artificial sequence
  Synthesized polypeptide
IYNGNSFPY

SEQ ID NO: 18 - protein, 17 aa, Artificial sequence
  Synthesized polypeptide
KSSQSLFNSGNQKNYLT

SEQ ID NO: 19 - protein, 7 aa, Artificial sequence
  Synthesized polypeptide
WASTRES

SEQ ID NO: 20 - protein, 9 aa, Artificial sequence
  Synthesized polypeptide
QNAYSFPYT

SEQ ID NO: 21 - protein, 118 aa, Artificial sequence
  Synthesized polypeptide
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSS

SEQ ID NO: 22 - protein, 114 aa, Artificial sequence
  Synthesized polypeptide
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 23 - protein, 243 aa, Artificial sequence
  Synthesized polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 24 - DNA, 729 bp, Artificial sequence
  Synthesized polynucleotide
gaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgt

SEQ ID NO: 25 - DNA, 1356 bp, Artificial sequence
  Synthesized polynucleotide
gaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgtgcggccgcaggtggcggcggttctggtggcggcggttctggtggcggcggttctctcgagatgcggtggaacactttctggggcatcctgtgcctcagcctcctagctgttggcacttgccaggacgatgccgagaacattgaatacaaagtctccatctcaggaaccagtgtagagttgacgtgccctctagacagtgacgagaacttaaaatgggaaaaaaatggccaagagctgcctcagaagcatgataagcacctggtgctccaggatttctcggaagtcgaggacagtggctactacgtctgctacacaccagcctcaaataaaaacacgtacttgtacctgaaagctcgagtgtgtgagtactgtgtggaggtggacctgacagcagtagccataatcatcattgttgacatctgtatcactctgggcttgctgatggtcatttattactggagcaagaataggaaggccaaggccaagcctgtgacccgaggaaccggtgctggtagcaggcccagagggcaaaacaaggagcggccaccacctgttcccaacccagactatgagcccatccgcaaaggccagcgggacctgtattctggcctgaatcagagagcagtc

SEQ ID NO: 26 - protein, 5 aa, Artificial sequence
  Synthesized polypeptide
DYEMH

SEQ ID NO: 27 - protein, 17 aa, Artificial sequence
  Synthesized polypeptide
AIHPGSGDTAYNQRFKG

SEQ ID NO: 28 - protein, 6 aa, Artificial sequence
  Synthesized polypeptide
FYSYAY

SEQ ID NO: 29 - protein, 16 aa, Artificial sequence
  Synthesized polypeptide
RSSQSLVHSNGNTYLQ

SEQ ID NO: 30 - protein, 7 aa, Artificial sequence
  Synthesized polypeptide
KVSNRFS

SEQ ID NO: 31 - protein, 9 aa, Artificial sequence
  Synthesized polypeptide
SQSIYVPYT

SEQ ID NO: 32 - protein, 115 aa, Artificial sequence
  Synthesized polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSA

SEQ ID NO: 33 - protein, 113 aa, Artificial sequence
  Synthesized polypeptide
DIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 34 - protein, 189 aa, Artificial sequence
  Synthesized polypeptide
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 35 - DNA, 567 bp, Artificial sequence
  Synthesized polynucleotide
atgcggtggaacactttctggggcatcctgtgcctcagcctcctagctgttggcacttgccaggacgatgccgagaacattgaatacaaagtctccatctcaggaaccagtgtagagttgacgtgccctctagacagtgacgagaacttaaaatgggaaaaaaatggccaagagctgcctcagaagcatgataagcacctggtgctccaggatttctcggaagtcgaggacagtggctactacgtctgctacacaccagcctcaaataaaaacacgtacttgtacctgaaagctcgagtgtgtgagtactgtgtggaggtggacctgacagcagtagccataatcatcattgttgacatctgtatcactctgggcttgctgatggtcatttattactggagcaagaataggaaggccaaggccaagcctgtgacccgaggaaccggtgctggtagcaggcccagagggcaaaacaaggagcggccaccacctgttcccaacccagactatgagcccatccgcaaaggccagcgggacctgtattctggcctgaatcagagagcagtc

SEQ ID NO: 36 - protein, 22 aa, Artificial sequence
  Synthesized polypeptide
VKQTLNFDLLKLAGDVESNPGP

SEQ ID NO: 37 - DNA, 66 bp, Artificial sequence
  Synthesized polynucleotide
gtgaaacagactttgaattttgaccttctgaagttggcaggagacgttgagtccaaccctgggccc

SEQ ID NO: 38 - protein, 133 aa, Artificial sequence
  Synthesized polypeptide
MAQMMTLSLLSLVLALCIPWTQGSDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG

SEQ ID NO: 39 - DNA, 399 bp, Artificial sequence
  Synthesized polynucleotide
atggctcagatgatgactctgagcctccttagcctggtcctggctctctgcatcccctggacccaaggcagtgatggagggggacaggactgctgccttaagtacagccagaagaaaattccctacagtattgtccgaggctataggaagcaagaaccaagtttaggctgtcccatcccggcaatcctgttcttaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagagga

SEQ ID NO: 40 - protein, 154 aa, Artificial sequence
  Synthesized polypeptide
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 41 - DNA, 462 bp, Artificial sequence
  Synthesized polynucleotide
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtata

SEQ ID NO: 42 - protein, 19 aa, Artificial sequence
  Synthesized polypeptide
ATNFSLLKQAGDVEENPGP

SEQ ID NO: 43 - DNA, 57 bp, Artificial sequence
  Synthesized polynucleotide
gctactaacttcagcctgctgaagcaggctggagacgtggaggagaaccctggacct

SEQ ID NO: 44 - protein, 62 aa, Artificial sequence
  Synthesized polypeptide
KRKICFIQKALRGKFVSYRRREENLFHTEGVKRKICFIQKALRGKFVSYRRREENLFHTEGV

SEQ ID NO: 45 - DNA, 192 bp, Artificial sequence
  Synthesized polynucleotide
aagaggaaaatttgtttcatacagaaggcgttaagaggaaaatttgtttcatacagaaggcgttaagaggaaaatttgtttcatacagaaggcgttaagaggaaaatttgtttcatacagaaggcgttaagaggaaaatttgtttcatacagaaggcgttaagaggaaaatttgtttcatacagaaggcgtt

SEQ ID NO: 46 - protein, 36 aa, Artificial sequence
  Synthesized polypeptide
NIVTPPYYFSSINSINCLPCRAAYHPCSLLTVTSSP

SEQ ID NO: 47 - DNA, 114 bp, Artificial sequence
  Synthesized polynucleotide
aacatcgtgacacccccatattatttttccagcattaacagtataaattgcctcccatgctgaagagctgcctatcacccttgctaatcactcctcacagtgacctcaagtcct

SEQ ID NO: 48 - protein, 542 aa, Artificial sequence
  Synthesized polypeptide
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 49 - DNA, 1626 bp, Artificial sequence
  Synthesized polynucleotide
atgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatcttgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 50 - protein, 409 aa, Artificial sequence
  Synthesized polypeptide
MRIPVDPSTSRRFTPPSTAFPCGGGGGGKMGENSGALSAQATAGPGGRTRPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKIEDQTKAFPDRFGDLRMRVTPSTPSPRGSLSTTSHFSSQAQTPIQGSSDLNPFSDPRQFDRSFPTLQSLTESRFPDPRMHYPGAMSAAFPYSATPSGTSLGSLSVAGMPASSRFHHTYLPPPYPGAPQSQSGPFQANPAPYHLFYGASSGSYQFSMAAAGGGERSPTRMLTSCPSGASVSAGNLMNPSLGQADGVEADGSHSNSPTALSTPGRMDEAVWRPY

SEQ ID NO: 51 - DNA, 1227 bp, Artificial sequence
  Synthesized polynucleotide
atgcgtattcccgtagacccgagcaccagccgccgcttcactcccccctccacggccttcccctgcggcggcggcggcggcggcaagatgggcgagaacagcggcgcgctaagcgcgcaggcaaccgcgggccccggcggccgcacccggcccgaagtgcgctcgatggtggacgtgctggccgaccacgcgggagagctcgtgcgcaccgacagccccaacttcctctgctccgtgctgccctcgcactggcgctgcaacaagacgctgccggtcgccttcaaggtggtggccctgggggatgtgccggatggaacggtggtgaccgtgatggccggcaatgatgagaactactccgccgagctgcgcaacgcttccgctgtcatgaagaaccaagtggccaggttcaacgaccttcgattcgtgggccgcagtgggcgagggaagagtttcacgctcacaatcaccgtgttcaccaaccctacccaagtggctacctaccaccgagccatcaaggtcactgtggatggaccccgggaaccccgacggcaccggcagaagatagaagaccagaccaaggccttccccgaccgctttggagacctgcgcatgcgtgtaacaccaagcacacccagcccccgtggctctctcagcaccacgagccacttcagcagccaggcccagaccccaatccaaggctcctcagacctgaaccccttctccgacccccgccagtttgaccgctccttccctacgctgcagagcctcacagagagccgcttcccggaccccaggatgcactacccgggagccatgtctgccgccttcccctacagcgccacaccatcgggcaccagcctgggcagcctgagcgtggcgggcatgccggccagcagccgcttccaccacacctacctccctccgccctaccccggggccccacagagccagagcgggccctttcaggccaaccccgcgccctaccacctcttttacggcgcctcctccggctcctaccagttctccatggcagccgcgggaggtggtgagcgctcgcccacccgcatgctgacctcctgccccagcggcgcttcggtgtcagcaggcaacctcatgaaccccagcctgggccaggctgatggcgtggaagccgacggcagccacagcaactcgcccacggccctgagcacgccgggccgcatggacgaggccgtgtggcggccctac

SEQ ID NO: 52 - protein, 17 aa, Artificial sequence
  Synthesized polypeptide
NKISLFSLHLCVGFLCE

SEQ ID NO: 53 - DNA, 51 bp, Artificial sequence
  Synthesized polynucleotide
aataaaatatctttattttcattacatctgtgtgttggttttttgtgtgag

SEQ ID NO: 54 - protein, 261 aa, Artificial sequence
  Synthesized polypeptide
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 55 - protein, 579 aa, Artificial sequence
  Synthesized polypeptide
MAGTVRTACLLVAMLLGLGCLGQAQPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQMMNPGLPESVLDINECLRGARRDLKVFGSFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNGGKLTTTIGKLCAHSQQRQYRSAYYPEDLFIDKKILKVAHVEHEETLSSRRRELIQKLKSFINFYSALPGYICSHSPVAENDTLCWNGQELVERYSQKAARNGMKNQFNLHELKMKGPEPVVSQIIDKLKHINQLLRTMSVPKGKVLDKSLDEEGLESGDCGDDEDECIGSSGDGMVKVKNQLRFLAELAYDLDVDDAPGNKQHGNQKDNEITTSHSVGNMPSPLKILISVAIYVACFFFLVH

SEQ ID NO: 56 - protein, 247 aa, Artificial sequence
  Synthesized polypeptide
QVQLQQSGAELARPGASVKMSCKASGYTFTSYTMHWVKQRPGQGLEWIGYINPSSGYTNYNQKFKDKATLTADKSSSTAYMQLSSLTSEDSAVYYCARIYYGNSFAYWGQGTTVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 57 - protein, 5 aa, Artificial sequence
  Synthesized polypeptide
SYTMH

SEQ ID NO: 58 - protein, 17 aa, Artificial sequence
  Synthesized polypeptide
YINPSSGYTNYNQKFKD

SEQ ID NO: 59 - protein, 9 aa, Artificial sequence
  Synthesized polypeptide
IYYGNSFAY

SEQ ID NO: 60 - protein, 17 aa, Artificial sequence
  Synthesized polypeptide
KSSQSLLNSGNQKNYLT

SEQ ID NO: 61 - protein, 7 aa, Artificial sequence
  Synthesized polypeptide
WASTRES

SEQ ID NO: 62 - protein, 9 aa, Artificial sequence
  Synthesized polypeptide
QNDYSYPLT

SEQ ID NO: 63 - protein, 118 aa, Artificial sequence
  Synthesized polypeptide
QVQLQQSGAELARPGASVKMSCKASGYTFTSYTMHWVKQRPGQGLEWIGYINPSSGYTNYNQKFKDKATLTADKSSSTAYMQLSSLTSEDSAVYYCARIYYGNSFAYWGQGTTVTVSS

SEQ ID NO: 64 - protein, 114 aa, Artificial sequence
  Synthesized polypeptide
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 65 - protein, 242 aa, Artificial sequence
  Synthesized polypeptide
QVQLQQSGPELVKPGALVKISCKASGYTFTSYDINWVKQRPGQGLEWIGWIYPGDGSTKYNEKFKGKATLTADKSSSTAYMQLSSLTSENSAVYFCARGGYRYDEAMDYWGQGTTVTVSSGGGGSGGGGSGGGGSQIVLTQSPAIMSASPGEKVTMTCSASSSISYMHWYQQKPGTSPKRWIYDTSKLASGVPARFSGSGSGTSYSLTISSMEAEDAATYYCHQRSSYPYTFGGGTKLEIKR

SEQ ID NO: 66 - protein, 5 aa, Artificial sequence
  Synthesized polypeptide
SYDIN

SEQ ID NO: 67 - protein, 17 aa, Artificial sequence
  Synthesized polypeptide
WIYPGDGSTKYNEKFKG

SEQ ID NO: 68 - protein, 11 aa, Artificial sequence
  Synthesized polypeptide
GGYRYDEAMDY

SEQ ID NO: 69 - protein, 10 aa, Artificial sequence
  Synthesized polypeptide
SASSSISYMH

SEQ ID NO: 70 - protein, 7 aa, Artificial sequence
  Synthesized polypeptide
DTSKLAS

SEQ ID NO: 71 - protein, 9 aa, Artificial sequence
  Synthesized polypeptide
HQRSSYPYT

SEQ ID NO: 72 - protein, 120 aa, Artificial sequence
  Synthesized polypeptide
QVQLQQSGPELVKPGALVKISCKASGYTFTSYDINWVKQRPGQGLEWIGWIYPGDGSTKYNEKFKGKATLTADKSSSTAYMQLSSLTSENSAVYFCARGGYRYDEAMDYWGQGTTVTVSS

SEQ ID NO: 73 - protein, 107 aa, Artificial sequence
  Synthesized polypeptide
QIVLTQSPAIMSASPGEKVTMTCSASSSISYMHWYQQKPGTSPKRWIYDTSKLASGVPARFSGSGSGTSYSLTISSMEAEDAATYYCHQRSSYPYTFGGGTKLEIKR

SEQ ID NO: 74 - protein, 247 aa, Artificial sequence
  Synthesized polypeptide
QIQLVQSGPELKKPGETVKISCKASGYTFTNYGMNWVKQAPGKGLKWMGWINTNTGEPTYAEEFKGRFAFSLETSASTAYLQINNLKNEDTATYFCARFSYGNSFAYWGQGTTVTVSSGGGGSGGGGSGGGGSDIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPLTFGAGTKLELKR

SEQ ID NO: 75 - protein, 5 aa, Artificial sequence
  Synthesized polypeptide
NYGMN

SEQ ID NO: 76 - protein, 17 aa, Artificial sequence
  Synthesized polypeptide
WINTNTGEPTYAEEFKG

SEQ ID NO: 77 - protein, 9 aa, Artificial sequence
  Synthesized polypeptide
FSYGNSFAY

SEQ ID NO: 78 - protein, 17 aa, Artificial sequence
  Synthesized polypeptide
KSSQSLLNSGNQKNYLA

SEQ ID NO: 79 - protein, 7 aa, Artificial sequence
  Synthesized polypeptide
GASTRES

SEQ ID NO: 80 - protein, 9 aa, Artificial sequence
  Synthesized polypeptide
QNDHSYPLT

SEQ ID NO: 81 - protein, 118 aa, Artificial sequence
  Synthesized polypeptide
QIQLVQSGPELKKPGETVKISCKASGYTFTNYGMNWVKQAPGKGLKWMGWINTNTGEPTYAEEFKGRFAFSLETSASTAYLQINNLKNEDTATYFCARFSYGNSFAYWGQGTTVTVSS

SEQ ID NO: 82 - protein, 114 aa, Artificial sequence
  Synthesized polypeptide
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPLTFGAGTKLELKR

SEQ ID NO: 83 - protein, 247 aa, Artificial sequence
  Synthesized polypeptide
DVQLQESGPDLVKPSQSLSLTCTVTGYSITSGYNWHWIRQFPGNKMEWMGYIHYTGSTNYNPSLRSRISITRDTSKNQFFLQLNSVTTDDTATYYCTRIYNGNSFPYWGQGTSVTVSSGGGGSGGGGSGGGGSDIVMTQSPSSLTVTPGEKVTMTCKSSQSLFNSGNQKNYLTWYQQRPGQPPKMLIYWASTRESGVPDRFTGSGSGTDFTLTISSVQAEDLAVFYCQNAYSFPYTFGGGTKLEIKR

SEQ ID NO: 84 - protein, 6 aa, Artificial sequence
  Synthesized polypeptide
SGYNWH

SEQ ID NO: 85 - protein, 16 aa, Artificial sequence
  Synthesized polypeptide
YIHYTGSTNYNPSLRS

SEQ ID NO: 86 - protein, 9 aa, Artificial sequence
  Synthesized polypeptide
IYNGNSFPY

SEQ ID NO: 87 - protein, 17 aa, Artificial sequence
  Synthesized polypeptide
KSSQSLFNSGNQKNYLT

SEQ ID NO: 88 - protein, 7 aa, Artificial sequence
  Synthesized polypeptide
WASTRES

SEQ ID NO: 89 - protein, 9 aa, Artificial sequence
  Synthesized polypeptide
QNAYSFPYT

SEQ ID NO: 90 - protein, 118 aa, Artificial sequence
  Synthesized polypeptide
DVQLQESGPDLVKPSQSLSLTCTVTGYSITSGYNWHWIRQFPGNKMEWMGYIHYTGSTNYNPSLRSRISITRDTSKNQFFLQLNSVTTDDTATYYCTRIYNGNSFPYWGQGTSVTVSS

SEQ ID NO: 91 - protein, 114 aa, Artificial sequence
  Synthesized polypeptide
DIVMTQSPSSLTVTPGEKVTMTCKSSQSLFNSGNQKNYLTWYQQRPGQPPKMLIYWASTRESGVPDRFTGSGSGTDFTLTISSVQAEDLAVFYCQNAYSFPYTFGGGTKLEIKR

SEQ ID NO: 92 - protein, 247 aa, Artificial sequence
  Synthesized polypeptide
QVQLQQSGAELARPGASVKMSCKASGYTFTSYTMHWVKQRPGQGLEWIGYIDPSSGYTNYNQKFKDKATLTADKSSSTAYMQLSSLTSEDSAVYYCARIYYGNSFAYWGQGTTVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 93 - protein, 5 aa, Artificial sequence
  Synthesized polypeptide
SYTMH

SEQ ID NO: 94 - protein, 17 aa, Artificial sequence
  Synthesized polypeptide
YIDPSSGYTNYNQKFKD

SEQ ID NO: 95 - protein, 9 aa, Artificial sequence
  Synthesized polypeptide
IYYGNSFAY

SEQ ID NO: 96 - protein, 17 aa, Artificial sequence
  Synthesized polypeptide
KSSQSLLNSGNQKNYLT

SEQ ID NO: 97 - protein, 7 aa, Artificial sequence
  Synthesized polypeptide
WASTRES

SEQ ID NO: 98 - protein, 9 aa, Artificial sequence
  Synthesized polypeptide
QNDYSYPLT

SEQ ID NO: 99 - protein, 118 aa, Artificial sequence
  Synthesized polypeptide
QVQLQQSGAELARPGASVKMSCKASGYTFTSYTMHWVKQRPGQGLEWIGYIDPSSGYTNYNQKFKDKATLTADKSSSTAYMQLSSLTSEDSAVYYCARIYYGNSFAYWGQGTTVTVSS

SEQ ID NO: 100 - protein, 114 aa, Artificial sequence; identical to SEQ ID NO: 64
  Synthesized polypeptide

SEQ ID NO: 101 - protein, 247 aa, Artificial sequence
  Synthesized polypeptide
QVQLQQSGAELARPGASVKMSCKASGYTFTSYTMHWVKQRPGQGLEWIGYINPASGYTNYNQKFKDKATLTADKSSSTAYMQLSSLTSEDSAVYYCARIYYGNSFAYWGQGTTVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 102 - protein, 5 aa, Artificial sequence
  Synthesized polypeptide
SYTMH

SEQ ID NO: 103 - protein, 17 aa, Artificial sequence
  Synthesized polypeptide
YINPASGYTNYNQKFKD

SEQ ID NO: 104 - protein, 9 aa, Artificial sequence
  Synthesized polypeptide
IYYGNSFAY

SEQ ID NO: 105 - protein, 17 aa, Artificial sequence
  Synthesized polypeptide
KSSQSLLNSGNQKNYLT

SEQ ID NO: 106 - protein, 7 aa, Artificial sequence
  Synthesized polypeptide
WASTRES

SEQ ID NO: 107 - protein, 9 aa, Artificial sequence
  Synthesized polypeptide
QNDYSYPLT

SEQ ID NO: 108 - protein, 118 aa, Artificial sequence
  Synthesized polypeptide
QVQLQQSGAELARPGASVKMSCKASGYTFTSYTMHWVKQRPGQGLEWIGYINPASGYTNYNQKFKDKATLTADKSSSTAYMQLSSLTSEDSAVYYCARIYYGNSFAYWGQGTTVTVSS

SEQ ID NO: 109 - protein, 114 aa, Artificial sequence; identical to SEQ ID NO: 64
  Synthesized polypeptide

SEQ ID NO: 110 - protein, 247 aa, Artificial sequence; identical to SEQ ID NO: 101
  Synthesized polypeptide

SEQ ID NO: 111 - protein, 5 aa, Artificial sequence
  Synthesized polypeptide
SYTMH

SEQ ID NO: 112 - protein, 17 aa, Artificial sequence
  Synthesized polypeptide
YINPASGYTNYNQKFKD

SEQ ID NO: 113 - protein, 9 aa, Artificial sequence
  Synthesized polypeptide
IYYGNSFAY

SEQ ID NO: 114 - protein, 17 aa, Artificial sequence
  Synthesized polypeptide
KSSQSLLNSGNQKNYLT

SEQ ID NO: 115 - protein, 7 aa, Artificial sequence
  Synthesized polypeptide
WASTRES

SEQ ID NO: 116 - protein, 9 aa, Artificial sequence
  Synthesized polypeptide
QNDYSYPLT

SEQ ID NO: 117 - protein, 118 aa, Artificial sequence; identical to SEQ ID NO: 108
  Synthesized polypeptide

SEQ ID NO: 118 - protein, 114 aa, Artificial sequence; identical to SEQ ID NO: 64
  Synthesized polypeptide

SEQ ID NO: 119 - protein, 247 aa, Artificial sequence
  Synthesized polypeptide
QVQLVQSGAEVKKPGASVKVSCKASGYTFTSYTMHWVRQAPGQGLEWMGYINPASGYTNYNQKFKDRVTMTRDTSTSTAYMELSSLRSEDTAVYYCARIYYGNSFAYWGQGTLVTVSSGGGGSGGGGSGGGGSDIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYSYPLTFGGGTKVEIKR

SEQ ID NO: 120 - protein, 5 aa, Artificial sequence
  Synthesized polypeptide
SYTMH

SEQ ID NO: 121 - protein, 17 aa, Artificial sequence
  Synthesized polypeptide
YINPASGYTNYNQKFKD

SEQ ID NO: 122 - protein, 9 aa, Artificial sequence
  Synthesized polypeptide
IYYGNSFAY

SEQ ID NO: 123 - protein, 17 aa, Artificial sequence
  Synthesized polypeptide
KSSQSLLNSGNQKNYLT

SEQ ID NO: 124 - protein, 7 aa, Artificial sequence
  Synthesized polypeptide
WASTRES

SEQ ID NO: 125 - protein, 9 aa, Artificial sequence
  Synthesized polypeptide
QNDYSYPLT

SEQ ID NO: 126 - protein, 118 aa, Artificial sequence
  Synthesized polypeptide
QVQLVQSGAEVKKPGASVKVSCKASGYTFTSYTMHWVRQAPGQGLEWMGYINPASGYTNYNQKFKDRVTMTRDTSTSTAYMELSSLRSEDTAVYYCARIYYGNSFAYWGQGTLVTVSS

SEQ ID NO: 127 - protein, 114 aa, Artificial sequence
  Synthesized polypeptide
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYSYPLTFGGGTKVEIKR

SEQ ID NO: 128 - protein, 247 aa, Artificial sequence
  Synthesized polypeptide
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 129 - protein, 6 aa, Artificial sequence
  Synthesized polypeptide
SGYNWH

SEQ ID NO: 130 - protein, 16 aa, Artificial sequence
  Synthesized polypeptide
YIHYTGSTNYNPALRS

SEQ ID NO: 131 - protein, 9 aa, Artificial sequence
  Synthesized polypeptide
IYNGNSFPY

SEQ ID NO: 132 - protein, 17 aa, Artificial sequence
  Synthesized polypeptide
KSSQSLFNSGNQKNYLT

SEQ ID NO: 133 - protein, 7 aa, Artificial sequence
  Synthesized polypeptide
WASTRES

SEQ ID NO: 134 - protein, 9 aa, Artificial sequence
  Synthesized polypeptide
QNAYSFPYT

SEQ ID NO: 135 - protein, 118 aa, Artificial sequence
  Synthesized polypeptide
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSS

SEQ ID NO: 136 - protein, 114 aa, Artificial sequence; identical to SEQ ID NO: 22
  Synthesized polypeptide

SEQ ID NO: 137 - protein, 247 aa, Artificial sequence
  Synthesized polypeptide
QVQLQESGGGLVQPGRSLRLSCAASGFTFSSYAMHWVRQAPGKGLEWVSAISGSGGSTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 138 - protein, 10 aa, Artificial sequence
  Synthesized polypeptide
GFTFSSYAMH

SEQ ID NO: 139 - protein, 17 aa, Artificial sequence
  Synthesized polypeptide
AISGSGGSTYYADSVKG

SEQ ID NO: 140 - protein, 12 aa, Artificial sequence
  Synthesized polypeptide
DRRGSHADAFDV

SEQ ID NO: 141 - protein, 14 aa, Artificial sequence
  Synthesized polypeptide
TGTSSDVGGYNYVS

SEQ ID NO: 142 - protein, 7 aa, Artificial sequence
  Synthesized polypeptide
GNSNRPS

SEQ ID NO: 143 - protein, 10 aa, Artificial sequence
  Synthesized polypeptide
QSYDSSLRVV

SEQ ID NO: 144 - protein, 121 aa, Artificial sequence
  Synthesized polypeptide
QVQLQESGGGLVQPGRSLRLSCAASGFTFSSYAMHWVRQAPGKGLEWVSAISGSGGSTYYADSVKGRFTISRDNSKNTLYLQMNSLRAEDTAVYYCAKDRRGSHADAFDVWGQGTLVTVSS

SEQ ID NO: 145 - protein, 111 aa, Artificial sequence
  Synthesized polypeptide
QSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 146 - protein, 247 aa, Artificial sequence
  Synthesized polypeptide
QVQLQESGGGLVQPGRSLRLSCAASGFTFSTYAMTWVRQAPGKGLEWVSSISSSGESTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 147 - protein, 10 aa, Artificial sequence
  Synthesized polypeptide
GFTFSTYAMT

SEQ ID NO: 148 - protein, 17 aa, Artificial sequence
  Synthesized polypeptide
SISSSGESTYYADSVKG

SEQ ID NO: 149 - protein, 12 aa, Artificial sequence
  Synthesized polypeptide
DRRGSHADAFDV

SEQ ID NO: 150 - protein, 14 aa, Artificial sequence
  Synthesized polypeptide
TGTSSDVGGYNYVS

SEQ ID NO: 151 - protein, 7 aa, Artificial sequence
  Synthesized polypeptide
GNSNRPS

SEQ ID NO: 152 - protein, 10 aa, Artificial sequence
  Synthesized polypeptide
QSYDSSLRVV

SEQ ID NO: 153 - protein, 121 aa, Artificial sequence
  Synthesized polypeptide
QVQLQESGGGLVQPGRSLRLSCAASGFTFSTYAMTWVRQAPGKGLEWVSSISSSGESTYYADSVKGRFTISRDNSKNTLYLQMNSLRAEDTAVYYCAKDRRGSHADAFDVWGQGTLVTVSS

SEQ ID NO: 154 - protein, 111 aa, Artificial sequence; identical to SEQ ID NO: 145
  Synthesized polypeptide

SEQ ID NO: 155 - protein, 247 aa, Artificial sequence
  Synthesized polypeptide
QVQLQESGGGLVQPGRSLRLSCAASGFTFSTYAMAWVRQAPGKGLEWVSEISSSGSRTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 156 - protein, 10 aa, Artificial sequence
  Synthesized polypeptide
GFTFSTYAMA

SEQ ID NO: 157 - protein, 17 aa, Artificial sequence
  Synthesized polypeptide
EISSSGSRTYYADSVKG

SEQ ID NO: 158 - protein, 12 aa, Artificial sequence
  Synthesized polypeptide
DRRGSHADAFDV

SEQ ID NO: 159 - protein, 14 aa, Artificial sequence
  Synthesized polypeptide
TGTSSDVGGYNYVS

SEQ ID NO: 160 - protein, 7 aa, Artificial sequence
  Synthesized polypeptide
GNSNRPS

SEQ ID NO: 161 - protein, 10 aa, Artificial sequence
  Synthesized polypeptide
QSYDSSLRVV

SEQ ID NO: 162 - protein, 121 aa, Artificial sequence
  Synthesized polypeptide
QVQLQESGGGLVQPGRSLRLSCAASGFTFSTYAMAWVRQAPGKGLEWVSEISSSGSRTYYADSVKGRFTISRDNSKNTLYLQMNSLRAEDTAVYYCAKDRRGSHADAFDVWGQGTLVTVSS

SEQ ID NO: 163 - protein, 111 aa, Artificial sequence; identical to SEQ ID NO: 145
  Synthesized polypeptide

SEQ ID NO: 164 - protein, 247 aa, Artificial sequence
  Synthesized polypeptide
QVQLQESGGGLVQPGRSLRLSCAASGFTFSTYAMNWVRQAPGKGLEWVSAISMSGESTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 165 - protein, 10 aa, Artificial sequence
  Synthesized polypeptide
GFTFSTYAMA

SEQ ID NO: 166 - protein, 17 aa, Artificial sequence
  Synthesized polypeptide
AISMSGESTYYADSVKG

SEQ ID NO: 167 - protein, 12 aa, Artificial sequence
  Synthesized polypeptide
DRRGSHADAFDV

SEQ ID NO: 168 - protein, 14 aa, Artificial sequence
  Synthesized polypeptide
TGTSSDVGGYNYVS

SEQ ID NO: 169 - protein, 7 aa, Artificial sequence
  Synthesized polypeptide
GNSNRPS

SEQ ID NO: 170 - protein, 10 aa, Artificial sequence
  Synthesized polypeptide
QSYDSSLRVV

SEQ ID NO: 171 - protein, 121 aa, Artificial sequence
  Synthesized polypeptide
QVQLQESGGGLVQPGRSLRLSCAASGFTFSTYAMNWVRQAPGKGLEWVSAISMSGESTYYADSVKGRFTISRDNSKNTLYLQMNSLRAEDTAVYYCAKDRRGSHADAFDVWGQGTLVTVSS

SEQ ID NO: 172 - protein, 111 aa, Artificial sequence; identical to SEQ ID NO: 145
  Synthesized polypeptide

SEQ ID NO: 173 - protein, 247 aa, Artificial sequence
  Synthesized polypeptide
QVQLQESGGGLVQPGRSLRLSCAASGFTFSSYAMHWVRQAPGKGLEWVSAISSSGGSTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGHKFPVSWYQQYPGKAPKLLIYKNLLRPSGVPDRFSGSKSGTSASLAITGLQAEDGADYYCQSYDSSLRVVFGGGTKVTVLG

SEQ ID NO: 174 - protein, 10 aa, Artificial sequence
  Synthesized polypeptide
GFTFSSYAMH

SEQ ID NO: 175 - protein, 17 aa, Artificial sequence
  Synthesized polypeptide
AISSSGGSTYYADSVKG

SEQ ID NO: 176 - protein, 12 aa, Artificial sequence
  Synthesized polypeptide
DRRGSHADAFDV

SEQ ID NO: 177 - protein, 14 aa, Artificial sequence
  Synthesized polypeptide
TGTSSDVGHKFPVS

SEQ ID NO: 178 - protein, 7 aa, Artificial sequence
  Synthesized polypeptide
KNLLRPS

SEQ ID NO: 179 - protein, 10 aa, Artificial sequence
  Synthesized polypeptide
QSYDSSLRVV

SEQ ID NO: 180 - protein, 121 aa, Artificial sequence
  Synthesized polypeptide
QVQLQESGGGLVQPGRSLRLSCAASGFTFSSYAMHWVRQAPGKGLEWVSAISSSGGSTYYADSVKGRFTISRDNSKNTLYLQMNSLRAEDTAVYYCAKDRRGSHADAFDVWGQGTLVTVSS

SEQ ID NO: 181 - protein, 111 aa, Artificial sequence
  Synthesized polypeptide
QSALTQPPSASGSPGQSVTISCTGTSSDVGHKFPVSWYQQYPGKAPKLLIYKNLLRPSGVPDRFSGSKSGTSASLAITGLQAEDGADYYCQSYDSSLRVVFGGGTKVTVLG

SEQ ID NO: 182 - protein, 247 aa, Artificial sequence
  Synthesized polypeptide
QVQLQESGGGLVQPGRSLRLSCAASGFTFSSYAMHWVRQAPGKGLEWVSAISSSGGSTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGLMHNVSWYQQYPGKAPKLLIYKSSSRPSGVPDRFSGSKSGTSASLAITGLQAEDGADYYCQSYDSSLRVVFGGGTKVTVLG

SEQ ID NO: 183 - protein, 10 aa, Artificial sequence
  Synthesized polypeptide
GFTFSSYAMH

SEQ ID NO: 184 - protein, 17 aa, Artificial sequence
  Synthesized polypeptide
AISSSGGSTYYADSVKG

SEQ ID NO: 185 - protein, 12 aa, Artificial sequence
  Synthesized polypeptide
DRRGSHADAFDV

SEQ ID NO: 186 - protein, 14 aa, Artificial sequence
  Synthesized polypeptide
TGTSSDVGLMHNVS

SEQ ID NO: 187 - protein, 7 aa, Artificial sequence
  Synthesized polypeptide
KSSSRPS

SEQ ID NO: 188 - protein, 10 aa, Artificial sequence
  Synthesized polypeptide
QSYDSSLRVV

SEQ ID NO: 189 - protein, 121 aa, Artificial sequence; identical to SEQ ID NO: 180
  Synthesized polypeptide

SEQ ID NO: 190 - protein, 111 aa, Artificial sequence
  Synthesized polypeptide
QSALTQPPSASGSPGQSVTISCTGTSSDVGLMHNVSWYQQYPGKAPKLLIYKSSSRPSGVPDRFSGSKSGTSASLAITGLQAEDGADYYCQSYDSSLRVVFGGGTKVTVLG

SEQ ID NO: 191 - protein, 247 aa, Artificial sequence
  Synthesized polypeptide
QVQLQESGGGLVQPGRSLRLSCAASGFTFSSYAMHWVRQAPGKGLEWVSAISSSGRSTYYADSVEGRFTISRDNSKNTLYLQMNSLRAEDTAVYYCAKDRRGSHADALNVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 192 - protein, 10 aa, Artificial sequence
  Synthesized polypeptide
GFTFSSYAMH

SEQ ID NO: 193 - protein, 17 aa, Artificial sequence
  Synthesized polypeptide
AISSSGRSTYYADSVEG

SEQ ID NO: 194 - protein, 12 aa, Artificial sequence
  Synthesized polypeptide
DRRGSHADALNV

SEQ ID NO: 195 - protein, 14 aa, Artificial sequence
  Synthesized polypeptide
TGTSSDVGGYNYVS

SEQ ID NO: 196 - protein, 7 aa, Artificial sequence
  Synthesized polypeptide
KSSSRPS

SEQ ID NO: 197 - protein, 10 aa, Artificial sequence
  Synthesized polypeptide
QSYDSSLRVV

SEQ ID NO: 198 - protein, 121 aa, Artificial sequence
  Synthesized polypeptide
QVQLQESGGGLVQPGRSLRLSCAASGFTFSSYAMHWVRQAPGKGLEWVSAISSSGRSTYYADSVEGRFTISRDNSKNTLYLQMNSLRAEDTAVYYCAKDRRGSHADALNVWGQGTLVTVSS

SEQ ID NO: 199 - protein, 111 aa, Artificial sequence; identical to SEQ ID NO: 145
  Synthesized polypeptide

SEQ ID NO: 200 - protein, 243 aa, Artificial sequence
  Synthesized polypeptide
QVQLQQSGTELVRPGASVKLSCKALGYTFTDYEMHWVKQTPVHGLEWIGAIHPGSGDTAYNQRFKGKATLTADKSSSTAYMEYSSLTSEDSAVYYCTRFYSYAYWGQGTLVTVSAGGGGSGGGGSGGGGSDVVMTQTPLSLPVSLGDQASISCRSSQSLVHSNGNTYLQWYLQKPGQSPKLLIYKVSNRFSGVPDRFSGRGSGTDFTLKISRVEAEDLGVYFCSQSIYVPYTFGGGTKLEIKR

SEQ ID NO: 201 - protein, 5 aa, Artificial sequence
  Synthesized polypeptide
DYEMH

SEQ ID NO: 202 - protein, 17 aa, Artificial sequence
  Synthesized polypeptide
AIHPGSGDTAYNQRFKG

SEQ ID NO: 203 - protein, 6 aa, Artificial sequence
  Synthesized polypeptide
FYSYAY

SEQ ID NO: 204 - protein, 16 aa, Artificial sequence
  Synthesized polypeptide
RSSQSLVHSNGNTYLQ

SEQ ID NO: 205 - protein, 7 aa, Artificial sequence
  Synthesized polypeptide
KVSNRFS

SEQ ID NO: 206 - protein, 10 aa, Artificial sequence
  Synthesized polypeptide
SQSIYVPYTF

SEQ ID NO: 207 - protein, 115 aa, Artificial sequence
  Synthesized polypeptide
QVQLQQSGTELVRPGASVKLSCKALGYTFTDYEMHWVKQTPVHGLEWIGAIHPGSGDTAYNQRFKGKATLTADKSSSTAYMEYSSLTSEDSAVYYCTRFYSYAYWGQGTLVTVSA

SEQ ID NO: 208 - protein, 113 aa, Artificial sequence
  Synthesized polypeptide
DVVMTQTPLSLPVSLGDQASISCRSSQSLVHSNGNTYLQWYLQKPGQSPKLLIYKVSNRFSGVPDRFSGRGSGTDFTLKISRVEAEDLGVYFCSQSIYVPYTFGGGTKLEIKR

(19) United States
(12) Patent Application Publication
(10) Pub. No.: US 2022/0127372 A1
(43) Pub. Date: Apr. 28, 2022

(54) TCR FUSION PROTEIN AND CELL EXPRESSING TCR FUSION PROTEIN

(71) Applicant: CAFA THERAPEUTICS LIMITED, Dublin (IE)

(72) Inventors: Zonghai LI, Shanghai (CN); Hua JIANG, Shanghai (CN); Peng WANG, Shanghai (CN)

(21) Appl. No.: 17/427,780

(22) PCT Filed: Feb. 2, 2020
(86) PCT No.: PCT/CN2020/074169

§ 371 Date: Aug. 2, 2021

(30) Foreign Application Priority Data
Feb. 1, 2019 (CN) 201910105233.0
Sep. 5, 2019 (CN) 201910838439.4

(51) Int. Cl. C07K 16/30; C07K 14/725; C07K 16/28; A61P 35/00

(52) U.S. Cl. CPC C07K 16/303; C07K 14/7051; C07K 16/28; A61K 2039/505; C07K 2319/02; C07K 2317/622; A61P 35/00

(57) ABSTRACT

Disclosed is a T cell receptor (TCR) fusion protein (TFP). The fusion protein comprises a TCR subunit (or referred to as a TCR unit) and an antigen recognition unit that recognizes an antigen. The antigen is GPC3 or claudin 18.2. Also disclosed is a T cell containing the fusion protein, a pharmaceutical composition and an application method of using the fusion protein or the T cell to treat diseases such as cancer. The use of TFP or T cells not only inhibits the growth of tumor cells, but also releases fewer cytokines, thereby effectively reducing the possibility of cytokine storms.

### TECHNICAL FIELD

The present invention relates to the field of immunotherapy. More particular, the present invention relates to a TCR fusion protein and cells expressing the TCR fusion protein.

### BACKGROUND

Currently, immunotherapy has become indispensable in the clinical treatment of tumors. Drugs and solutions used in immunotherapy involve various stages of the body's immune system to recognize and attack cancer cells. Existing tumor immune drugs include following types: antibodies targeting cancer cells, adoptive cell therapy, oncolytic viruses, dendritic cell-related therapies, tumor vaccines at the DNA and protein levels, immune-activating cytokines, and other immune regulatory compounds, among which, antibody drugs against T cell checkpoint-inhibiting proteins and tumor antigen-specific T cell adoptive therapy have achieved breakthroughs in recent years and have attracted wide attention.

Genetic engineering based on T cells includes CAR-T and TCR-T. The former requires the construction of a chimeric antigen receptor, usually by connecting a single-chain antibody to the intracellular segment of CD3ζ through a hinge region and a transmembrane segment, and then the chimeric antigen receptor will be transduced into T cells through virus, the single-chain antibody binds to the antigen on the tumor cell surface so as to activate the intracellular signal of CD3ζ, which in turn causes the transduced T cells to kill tumor cells. The latter usually requires the transduction of a TCR that can specifically recognize the tumor antigen peptide/MHC complex into T cells, and then use these TCRs to form a new TCR complex with the CD3 subunit inside the T cell, so that the T cell can specifically target tumor cells, activate the signal pathway of the entire TCR complex, and achieve the purpose of killing tumor cells.

The method described herein is a new modification for T cells, which fuses a single-chain antibody with CD3ε or CD3γ, and then forms a new TCR complex with other subunits of the endogenous TCR complex, thereby both using the targeting properties of the single-chain antibody and the entire signaling pathway of the TCR complex.

### SUMMARY OF THE INVENTION

The purpose of the present invention is to provide a TCR fusion protein and T cells expressing the TCR fusion protein, so as to improve application effects of tumor immunotherapy in solid tumors.

The technical solution adopted in the present invention is as follows:

In the first aspect, the present invention provides a T cell receptor (TCR) fusion protein (TFP), the fusion protein comprising:

(a) a TCR subunit (or a TCR unit); and

(b) an antigen recognition unit that recognizes the antigen;

the antigen is GPC3 or claudin 18.2;

wherein the TCR subunit and the antigen recognition unit are operably connected.

In a specific embodiment, the TCR subunit comprises:

(i) at least a part of the extracellular domain of TCR, and

(ii) a TCR intracellular domain comprising a stimulatory domain derived from an intracellular signaling domain of CD3ε, CD3γ, CD3δ, TCRα, or TCRβ.

In a preferred embodiment, the TFP is incorporated into the TCR when expressed in T cells.

In a specific embodiment, the light chain LCDR1, LCDR2, and LCDR3 of the amino acid sequence of the antigen recognition unit that recognizes GPC3 are independently selected from or have 70-100% sequence identity with the light chain LCDR1, LCDR2, and LCDR3 shown in the following table, and/or the heavy chain HCDR1, HCDR2 and HCDR3 of the amino acid sequence of the antigen recognition unit that recognizes GPC3 are independently selected from or have 70-100% sequence identity with the heavy chain HCDR1, HCDR2 and HCDR3 shown in the following table:

| LCDR1 | LCDR2 | LCDR3 | HCDR1 | HCDR2 | HCDR3 |
|---|---|---|---|---|---|
| TGTSSDVGGYNYVS | GNSNRP | QSYDSSLRV | GFTFSSYAM | AISGSGGSTYYADSVK | DRRGSHADAFD |
| | S | V | H | G | V |
| TGTSSDVGGYNYVS | GNSNRP | QSYDSSLRV | GFTFSTYAMT | SISSSGESTYYADSVKG | DRRGSHADAFD |
| | S | V | | | V |
| TGTSSDVGGYNYVS | GNSNRP | QSYDSSLRV | GFTFSTYAM | EISSSGSRTYYADSVKG | DRRGSHADAFD |
| | S | V | A | | V |
| TGTSSDVGGYNYVS | GNSNRP | QSYDSSLRV | GFTFSTYAM | AISMSGESTYYADSVK | DRRGSHADAFD |
| | S | V | A | G | V |
| TGTSSDVGHKFPVS | KNLLRP | QSYDSSLRV | GFTFSSYAM | AISSSGGSTYYADSVKG | DRRGSHADAFD |
| | S | V | H | | V |
| TGTSSDVGLMHNVS | KSSSRPS | QSYDSSLRV | GFTFSSYAM | AISSSGGSTYYADSVKG | DRRGSHADAFD |
| | | V | H | | V |
| TGTSSDVGGYNYVS | KSSSRPS | QSYDSSLRV | GFTFSSYAM | AISSSGRSTYYADSVEG | DRRGSHADALN |
| | | V | H | | V |
| RSSQSLVHSNGNTYL | KVSNRF | SQSIYVPYT | DYEMH | AIHPGSGDTAYNQRFK | FYSYAY |
| Q | S | | | G | |
| RSSQSLVHSNGNTYL | KVSNRF | SQSIYVPYTF | DYEMH | AIHPGSGDTAYNQRFK | FYSYAY |
| Q | S | | | G | |

or

the light chain LCDR1, LCDR2, and LCDR3 of the amino acid sequence of the antigen recognition unit that recognizes claudin 18.2 are independently selected from or have 70-100% sequence identity with the light chain LCDR1, LCDR2, and LCDR3 shown in the following table, and/or the heavy chain HCDR1, HCDR2 and HCDR3 of the amino acid sequence of the antigen recognition unit that recognizes claudin 18.2 are independently selected from or have 70-100% sequence identity with the heavy chain HCDR1, HCDR2 and HCDR3 shown in the following table;

| LCDR1 | LCDR2 | LCDR3 | HCDR1 | HCDR2 | HCDR3 |
|---|---|---|---|---|---|
| KSSQSLLNSGNQKNYLT | WASTRES | QNDYSYPLT | SYTMH | YINPSSGYTNYNQKFKD | IYYGNSFAY |
| SASSSISYMH | DTSKLAS | HQRSSYPYT | SYDIN | WIYPGDGSTKYNEKFKG | GGYRYDEAMDY |
| KSSQSLLNSGNQKNYLA | GASTRES | QNDHSYPLT | NYGMN | WINTNTGEPTYAEEFKG | FSYGNSFAY |
| KSSQSLFNSGNQKNYLT | WASTRES | QNAYSFPYT | SGYNWH | YIHYTGSTNYNPSLRS | IYNGNSFPY |
| KSSQSLLNSGNQKNYLT | WASTRES | QNDYSYPLT | SYTMH | YIDPSSGYTNYNQKFKD | IYYGNSFAY |
| KSSQSLLNSGNQKNYLT | WASTRES | QNDYSYPLT | SYTMH | YINPASGYTNYNQKFKD | IYYGNSFAY |
| KSSQSLLNSGNQKNYLT | WASTRES | QNDYSYPLT | SYTMH | YINPASGYTNYNQKFKD | IYYGNSFAY |
| KSSQSLLNSGNQKNYLT | WASTRES | QNDYSYPLT | SYTMH | YINPASGYTNYNQKFKD | IYYGNSFAY |
| KSSQSLFNSGNQKNYLT | WASTRES | QNAYSFPYT | SGYNWH | YIHYTGSTNYNPALRS | IYNGNSFPY |
| KSSQSLFNSGNQKNYLT | WASTRES | QNAYSFPYT | SGYNWH | YIHYTGSTNYNPALRS | IYNGNSFPY. |

In a specific embodiment, the light chain variable region of the amino acid sequence of the antigen recognition unit that recognizes GPC3 is independently selected from or has 70-100% sequence identity with the light chain variable regions shown in the following table, and/or the heavy chain variable region of the amino acid sequence of the antigen recognition unit that recognizes GPC3 is independently selected from or has 70-100% sequence identity with the heavy chain variable region shown in the following table;

| VH | VL |
|---|---|
| QVQLQESGGGLVQPGRSLRLSCAASGFTFSSYAMHWVRQA | QSALTQPPSASGSPGQSVTISCTGTSSDVGGYNYVSW |
| PGKGLEWVSAISGSGGSTYYADSVKGRFTISRDNSKNTLYL | YQQYPGKAPKWYGNSNRPSGVPDRFSGSKSGTSAS |
| QMNSLRAEDTAVYYCAKDRRGSHADAFDVWGQGTLVTVS | LAITGLQAEDGADYYCQSYDSSLRVVFGGGTKVTVL |
| S | G |
| QVQLQESGGGLVQPGRSLRLSCAASGFTFSTYAMTWVRQA | QSALTQPPSASGSPGQSVTISCTGTSSDVGGYNYVSW |
| PGKGLEWVSSISSSGESTYYADSVKGRFTISRDNSKNTLYLQ | YQQYPKAPKWYGNSNRPSGVPDRFSGSKSGTSAS |
| MNSLRAEDTAVYYCAKDRRGSHADAFDVWGQGTLVTVSS | LAITGLQAEDGADYYCQSYDSSLRVVFGGGTKVTVL |
| | G |
| QVQLQESGGGLVQPGRSLRLSCAASGFTFSTYAMAWVRQA | QSALTQPPSASGSPGQSVTISCTGTSSDVGGYNYVSW |
| PGKGLEWVSEISSSGSRTYYADSVKGRFTISRDNSKNTLYLQ | YQQYPGKAPKLLIYGNSNRPSGVPDRFSGSKSGTSAS |
| MNSLRAEDTAVYYCAKDRRGSHADAFDVWGQGTLVTVSS | LAITGLQAEDGADYYCQSYDSSLRVVFGGGTKVTVL |
| | G |
| QVQLQESGGGLVQPGRSLRLSCAASGFTFSTYAMNWVRQA | QSALTQPPSASGSPGQSVTISCTGTSSDVGGYNYVSW |
| PGKGLEWVSAISMSGESTYYADSVKGRFTISRDNSKNTLYL | YQQYPGKAPKWYGNSNRPSGVPDRFSGSKSGTSAS |
| QMNSLRAEDTAVYYCAKDRRGSHADAFDVWGQGTLVTVS | LAITGLQAEDGADYYCQSYDSSLRVVFGGGTKVTVL |
| S | G |
| QVQLQESGGGLVQPGRSLRLSCAASGFTFSSYAMHWVRQA | QSALTQPPSASGSPGQSVTISCTGTSSDVGHKFPVSWY |
| PGKGLEWVSAISSSGGSTYYADSVKGRFTISRDNSKNTLYLQ | QQYPGKAPKLLIYKNLLRPSGVPDRFSGSKSGTSASLA |
| MNSLRAEDTAVYYCAKDRRGSHADAFDVWGQGTLVTVSS | ITGLQAEDGADYYCQSYDSSLRVVFGGGTKVTVLG |
| QVQLQESGGGLVQPGRSLRLSCAASGFTFSSYAMHWVRQA | QSALTQPPSASGSPGQSVTISCTGTSSDVGLMHNVSW |
| PGKGLEWVSAISSSGGSTYYADSVKGRFTISRDNSKNTLYLQ | YQQYPGKAPKLLIYKSSSRPSGVPDRFSGSKSGTSASL |
| MNSLRAEDTAVYYCAKDRRGSHADAFDVWGQGTLVTVSS | AITGLQAEDGADYYCQSYDSSLRVVFGGGTKVTVLG |
| QVQLQESGGGLVQPGRSLRLSCAASGFTFSSYAMHWVRQA | QSALTQPPSASGSPGQSVTISCTGTSSDVGGYNYVSW |
| PGKGLEWVSAISSSGRSTYYADSVEGRFTISRDNSKNTLYLQ | YQQYPGKAPKWYGNSNRPSGVPDRFSGSKSGTSAS |
| MNSLRAEDTAVYYCAKDRRGSHADALNVWGQGTLVTVSS | LAITGLQAEDGADYYCQSYDSSLRVVFGGGTKVTVL |
| | G |
| EVQLVQSGAEVKKPGASVKVSCKASGYTFSDYEMHWVRQ | DIVMTQTPLSLPVTPGEPASISCRSSQSLVHSNGNTYL |
| APGQGLEWMGAIHPGSGDTAYNQRFKGRVTITADKSTSTAY | QWYLQKPGQSPQLLIYKVSNRFSGVPDRFSGSGSGTD |
| MELSSLRSEDTAVYYCARFYSYAYWGQGTLVTVSA | FTLKISRVEAEDVGVYYCSQSIYVPYTFGQGTKLEIKR |
| QVQLQQSGTELVRPGASVKLSCKALGYTFTDYEMHWVKQT | DVVMTQTPLSLPVSLGDQASISCRSSQSLVHSNGNTY |
| PVHGLEWIGAIHPGSGDTAYNQRFKGKATLTADKSSSTAYM | LQWYLQKPGQSPKLLIYKVSNRFSGVPDRFSGRGSGT |
| EYSSLTSEDSAVYYCTRFYSYAYWGQGTLVTVSA | DFTLKISRVEAEDLGVYFCSQSIYVPYTFGGGTKLEIK |
| | R |

or,

the light chain variable region of the amino acid sequence of the antigen recognition unit that recognizes claudin18.2 is independently selected from or has 70-100% sequence identity with the light chain variable regions shown in the following table, and/or the heavy chain variable region of the amino acid sequence of the antigen recognition unit that recognizes claudin18.2 is independently selected from or has 70-100% sequence identity with the heavy chain variable region shown in the following table;

| VH | VL |
|---|---|
| QVQLQQSGAELARPGASVKMSCKASGYTFTSYTMHWVKQ | DIVMTQSPSSLTVTAGEKVTMSCKSSQSLLNSGNQKNY |
| RPGQGLEWIGYINPSSGYTNYNQKFKDKATLTADKSSSTA | LTWYQQKPGQPPKLLIYWASTRESGVPDRFTGSGSGTD |
| YMQLSSLTSEDSAVYYCARIYYGNSFAYWGQGTTVTVSS | FTLTISSVQAEDLAVYYCQNDYSYPLTFGAGTKLELKR |
| QVQLQQSGPELVKPGALVMSCKASGYTFTSYDINWVKQR | QIVLTQSPAIMSASPGEKVTMTCSASSSISYMHWYQQK |
| PGQGLEWIGWIYPGDGSTKYNEKFKGKATLTADKSSSTAY | PGTSPKRWIYDTSKLASGVPARFSGSGSGTSYSLTISSM |
| MQLSSLTSENSAVYFCARGGYRYDEAMDYWGQGTTVTVS | EAEDAATYYCHQRSSYPYTFGGGTKLEIKR |
| S | |
| QIQLVQSGPELKKPGETVKISCKASGYTFTNYGMNWVKQA | DIVMTQSPSSLSVSAGEKVTMSCKSSQSLLNSGNQKNY |
| PGKGLKWMGWINTNTGEPTYAEEFKGRFAFSLETSASTAY | LAWYQQKPGQPPKLLIYGASTRESGVPDRFTGSGSGTD |
| LQINNLKNEDTATYFCARFSYGNSFAYWGQGTTVTVSS | FTLTISSVQAEDLAVYYCQNDHSYPLTFGAGTKLELKR |
| DVQLQESGPDLVKPSQSLSLTCTVTGYSITSGYNWHWIRQF | DIVMTQSPSSLTVTPGEKVTMTCKSSQSLFNSGNQKNY |
| PGNKMEWMGYIHYTGSTNYNPSLRSR1SITRDTSKNQFFLQ | LTWYQQRPGQPPKMLIYWASTRESGVPDRFTGSGSGT |
| LNSVTTDDTATYYCTRIYNGNSFPYWGQGTSVTVSS | DFTLTISSVQAEDLAVFYCQNAYSFPYTFGGGTKLEIKR |
| QVQLQQSGAELARPGASVKMSCKASGYTFTSYTMHWVKQ | DIVMTQSPSSLTVTAGEKVTMSCKSSQSLLNSGNQKNY |
| RPGQGLEWIGYIDPSSGYTNYNQKFKDKATLTADKSSSTA | LTWYQQKPGQPPKLLIYWASTRESGVPDRFTGSGSGTD |
| YMQLSSLTSEDSAVYYCARIYYGNSFAYWGQGTTVTVSS | FTLTISSVQAEDLAVYYCQNDYSYPLTFGAGTKLELKR |
| QVQLQQSGAELARPGASVKMSCKASGYTFTSYTMHWVKQ | DIVMTQSPSSLTVTAGEKVTMSCKSSQSLLNSGNQKNY |
| RPGQGLEWIGYINPASGYTNYNQKFKDKATLTADKSSSTA | LTWYQQKPGQPPKLLIYWASTRESGVPDRFTGSGSGTD |
| YMQLSSLTSEDSAVYYCARIYYGNSFAYWGQGTTVTVSS | FTLTISSVQAEDLAVYYCQNDYSYPLTFGAGTKLELKR |
| QVQLQQSGAELARPGASVKMSCKASGYTFTSYTMHWVKQ | DIVMTQSPSSLTVTAGEKVTMSCKSSQSLLNSGNQKNY |
| RPGQGLEWIGYINPASGYTNYNQKFKDKATLTADKSSSTA | LTWYQQKPGQPPKLLIYWASTRESGVPDRFTGSGSGTD |
| YMQLSSLTSEDSAVYYCARIYYGNSFAYWGQGTTVTVSS | FTLTISSVQAEDLAVYYCQNDYSYPLTFGAGTKLELKR |
| QVQLVQSGAEVKKPGASVKVSCKASGYTFTSYTMHWVRQ | DIVMTQSPDSLAVSLGERATINCKSSQSLLNSGNQKNY |
| APGQGLEWMGYINPASGYTNYNQKFKDRVTMTRDTSTST | LTWYQQKPGQPPKLLIYWASTRESGVPDRFSGSGSGTD |
| AYMELSSLRSEDTAVYYCARIYYGNSFAYWGQGTLVTVSS | FTLTISSLQAEDVAVYYCQNDYSYPLTFGGGTKVEIKR |
| QVQLQESGPGLVKPSQTLSLTCTVSGGSISSGYNWHWIRQP | DIVMTQSPDSLAVSLGERATINCKSSQSLFNSGNQKNY |
| PGKGLEWIGYIHYTGSTNYNPALRSRVTISVDTSKNQFSLK | LTWYQQKPGQPPKLLIYWASTRESGVPDRFSGSGSGTD |
| LSSVTAADTAVYYCARlYNGNSFPYWGQGTTVTVSS | FTLTISSLQAEDVAVYYCQNAYSFPYTFGGGTKLEIKR |
| QVQLQESGPGLIKPSQTLSLTCTVSGGSISSGYNWHWIRQPP | DIVMTQSPDSLAVSLGERATINCKSSQSLFNSGNQKNY |
| GKGLEWIGYIHYTGSTNYNPALRSRVTISVDTSKNQFSLKL | LTWYQQKPGQPPKLLIYWASTRESGVPDRFSGSGSGTD |
| SSVTAADTAIYYCARIYNGNSFPYWGQGTTVTVSS | FTLTISSLQAEDVAVYYCQNAYSFPYTFGGGTKLEIKR. |

In a specific embodiment, the light chain LCDR1, LCDR2, and LCDR3 of the amino acid sequence of the antigen recognition unit that recognizes GPC3 are or have 70-100% sequence identity with a combination of the light chain LCDR1, LCDR2, and LCDR3 shown in any row of the following table, and/or the heavy chain HCDR1, HCDR2, and HCDR3 of the amino acid sequence of the antigen recognition unit that recognizes GPC3 are or have 70-100% sequence identity with a combination of the heavy chain HCDR1, HCDR2, and HCDR3 shown in any row of the following table:

| LCDR1 | LCDR2 | LCDR3 | HCDR1 | HCDR2 | HCDR3 |
|---|---|---|---|---|---|
| TGTSSDVGGYNYVS | GNSNRP | QSYDSSLRV | GFTFSSYAM | AISGSGGSTYYADSVK | DRRGSHADAFD |
| | S | V | H | G | V |
| TGTSSDVGGYNYVS | GNSNRP | QSYDSSLRV | GFTFSTYAMT | SISSSGESTYYADSVKG | DRRGSHADAFD |
| | S | V | | | V |
| TGTSSDVGGYNYVS | GNSNRP | QSYDSSLRV | GFTFSTYAM | EISSSGSRTYYADSVKG | DRRGSHADAFD |
| | S | V | A | | V |
| TGTSSDVGGYNYVS | GNSNRP | QSYDSSLRV | GFTFSTYAM | AISMSGESTYYADSVK | DRRGSHADAFD |
| | S | V | A | G | V |
| TGTSSDVGHKFPVS | KNLLRP | QSYDSSLRV | GFTFSSYAM | AISSSGGSTYYADSVKG | DRRGSHADAFD |
| | S | V | H | | V |
| TGTSSDVGLMHNVS | KSSSRPS | QSYDSSLRV | GFTFSSYAM | AISSSGGSTYYADSVKG | DRRGSHADAFD |
| | | V | H | | V |
| TGTSSDVGGYNYVS | KSSSRPS | QSYDSSLRV | GFTFSSYAM | AISSSGRSTYYADSVEG | DRRGSHADALN |
| | | V | H | | V |
| RSSQSLVHSNGNTYL | KVSNRF | SQSIYVPYT | DYEMH | AIHPGSGDTAYNQRFK | FYSYAY |
| Q | S | | | G | |
| RSSQSLVHSNGNTYL | KVSNRF | SQSIYVPYTF | DYEMH | AIHPGSGDTAYNQRFK | FYSYAY; |
| Q | S | | | G | |

or

the light chain LCDR1, LCDR2, and LCDR3 of the amino acid sequence of the antigen recognition unit that recognizes claudin18.2 are or have 70-100% sequence identity with a combination of the light chain LCDR1, LCDR2, and LCDR3 shown in any row of the following table, and/or the heavy chain HCDR1, HCDR2, and HCDR3 of the amino acid sequence of the antigen recognition unit that recognizes claudin18.2 are or have 70-100% sequence identity with a combination of the heavy chain HCDR1, HCDR2, and HCDR3 shown in any row of the following table:

| LCDR1 | LCDR2 | LCDR3 | HCDR1 | HCDR2 | HCDR3 |
|---|---|---|---|---|---|
| KSSQSLLNSGNQKNYLT | WASTRES | QNDYSYPLT | SYTMH | YINPSSGYTNYNQKFKD | IYYGNSFAY |
| SASSSISYMH | DTSKLAS | HQRSSYPYT | SYDIN | WIYPGDGSTKYNEKFKG | GGYRYDEAMDY |
| KSSQSLLNSGNQKNYLA | GASTRES | QNDHSYPLT | NYGMN | WINTNTGEPTYAEEFKG | FSYGNSFAY |
| KSSQSLFNSGNQKNYLT | WASTRES | QNAYSFPYT | SGYNWH | YIHYTGSTNYNPSLRS | IYNGNSFPY |
| KSSQSLLNSGNQKNYLT | WASTRES | QNDYSYPLT | SYTMH | YIDPSSGYTNYNQKFKD | IYYGNSFAY |
| KSSQSLLNSGNQKNYLT | WASTRES | QNDYSYPLT | SYTMH | YINPASGYTNYNQKFKD | IYYGNSFAY |
| KSSQSLLNSGNQKNYLT | WASTRES | QNDYSYPLT | SYTMH | YINPASGYTNYNQKFKD | IYYGNSFAY |
| KSSQSLLNSGNQKNYLT | WASTRES | QNDYSYPLT | SYTMH | YINPASGYTNYNQKFKD | IYYGNSFAY |
| KSSQSLFNSGNQKNYLT | WASTRES | QNAYSFPYT | SGYNWH | YIHYTGSTNYNPALRS | IYNGNSFPY |
| KSSQSLFNSGNQKNYLT | WASTRES | QNAYSFPYT | SGYNWH | YIHYTGSTNYNPALRS | IYNGNSFPY. |

In a specific embodiment, the light chain variable region and the heavy chain variable region of the amino acid sequence of the antigen recognition unit that recognizes GPC3 are or have 70-100% sequence identity with a combination of the light chain variable region and the heavy chain variable region shown in any row of the following table:

| VH | VL |
|---|---|
| QVQLQESGGGLVQPGRSLRLSCAASGFTFSSYAMHWVRQA | QSALTQPPSASGSPGQSVTISCTGTSSDVGGYNYVSWY |
| PGKGLEWVSAISGSGGSTYYADSVKGRFTISRDNSKNTLYL | QQYPGKAPKLLIYGNSNRPSGVPDRFSGSKSGTSASLA |
| QMNSLRAEDTAVYYCAKDRRGSHADAFDVWGQGTLVTVS | ITGLQAEDGADYYCQSYDSSLRVVFGGGTKVTVLG |
| S | |
| QVQLQESGGGLVQPGRSLRLSCAASGFTFSTYAMTWVRQA | QSALTQPPSASGSPGQSVTISCTGTSSDVGGYNYVSWY |
| PGKGLEWVSSISSSGESTYYADSVKGRFTISRDNSKNTLYLQ | QQYPGKAPKLLIYGNSNRPSGVPDRFSGSKSGTSASLA |
| MNSLRAEDTAVYYCAKDRRGSHADAFDVWGQGTLVTVSS | ITGLQAEDGADYYCQSYDSSLRVVFGGGTKVTVLG |
| QVQLQESGGGLVQPGRSLRLSCAASGFTFSTYAMAWVRQA | QSALTQPPSASGSPGQSVTISCTGTSSDVGGYNYVSWY |
| PGKGLEWVSEISSSGSRTYYADSVKGRFTISRDNSKNTLYLQ | QQYPGKAPKLLIYGNSNRPSGVPDRFSGSKSGTSASLA |
| MNSLRAEDTAVYYCAKDRRGSHADAFDVWGQGTLVTVSS | ITGLQAEDGADYYCQSYDSSLRVVFGGGTKVTVLG |
| QVQLQESGGGLVQPGRSLRLSCAASGFTFSTYAMNWVRQA | QSALTQPPSASGSPGQSVTISCTGTSSDVGGYNYVSWY |
| PGKGLEWVSAISMSGESTYYADSVKGRFTISRDNSKNTLYL | QQYPGKAPKLLIYGNSNRPSGVPDRFSGSKSGTSASLA |
| QMNSLRAEDTAVYYCAKDRRGSHADAFDVWGQGTLVTVS | ITGLQAEDGADYYCQSYDSSLRVVFGGGTKVTVLG |
| S | |
| QVQLQESGGGLVQPGRSLRLSCAASGFTFSSYAMHWVRQA | QSALTQPPSASGSPGQSVTISCTGTSSDVGHKFPVSWY |
| PGKGLEWVSAISSSGGSTYYADSVKGRFTISRDNSKNTLYL | QQYPGKAPKLLIYKNLLRPSGVPDRFSGSKSGTSASLA |
| QMNSLRAEDTAVYYCAKDRRGSHADAFDVWGQGTLVTVS | ITGLQAEDGADYYCQSYDSSLRVVFGGGTKVTVLG |
| S | |
| QVQLQESGGGLVQPGRSLRLSCAASGFTFSSYAMHWVRQA | QSALTQPPSASGSPGQSVTISCTGTSSDVGLMHNVSW |
| PGKGLEWVSAISSSGGSTYYADSVKGRFTISRDNSKNTLYL | YQQYPGKAPKLLIYKSSSRPSGVPDRFSGSKSGTSASL |
| QMNSLRAEDTAVYYCAKDRRGSHADAFDVWGQGTLVTVS | AITGLQAEDGADYYCQSYDSSLRVVFGGGTKVTVLG |
| S | |
| QVQLQESGGGLVQPGRSLRLSCAASGFTFSSYAMHWVRQA | QSALTQPPSASGSPGQSVTISCTGTSSDVGGYNYVSWY |
| PGKGLEWVSAISSSGRSTYYADSVEGRFTISRDNSKNTLYLQ | QQYPGKAPKLLIYGNSNRPSGVPDRFSGSKSGTSASLA |
| MNSLRAEDTAVYYCAKDRRGSHADALNVWGQGTLVTVSS | ITGLQAEDGADYYCQSYDSSLRVVFGGGTKVTVLG |
| EVQLVQSGAEVKKPGASVKVSCKASGYTFSDYEMHWVRQ | DIVMTQTPLSLPVTPGEPASISCRSSQSLVHSNGNTYLQ |
| APGQGLEWMGAIHPGSGDTAYNQRFKGRVTITADKSTSTA | WYLQKPGQSPQLLIYKVSNRFSGVPDRFSGSGSGTDFT |
| YMELSSLRSEDTAVYYCARFYSYAYWGQGTLVTVSA | LKISRVEAEDVGVYYCSQSIYVPYTFGQGTKLEIKR |
| QVQLQQSGTELVRPGASVKLSCKALGYTFTDYEMHWVKQ | DVVMTQTPLSLPVSLGDQASISCRSSQSLVHSNGNTYL |
| TPVHGLEWIGAIHPGSGDTAYNQRFKGKATLTADKSSSTAY | QWYLQKPGQSPKLLIYKVSNRFSGVPDRFSGRGSGTD |
| MEYSSLTSEDSAVYYCTRFYSYAYWGQGTLVTVSA | FTLKISRVEAEDLGVYFCSQSIYVPYTFGGGTKLEIKR; |

or,

the light chain variable region and the heavy chain variable region of the amino acid sequence of the antigen recognition unit that recognizes claudin 18.2 are or have 70-100% sequence identity with a combination of the light chain variable region and the heavy chain variable region shown in any row of the following table;

| VH | VL |
|---|---|
| QVQLQQSGAELARPGASVKMSCKASGYTFTSYTMHWVKQ | DIVMTQSPSSLTVTAGEKVTMSCKSSQSLLNSGNQK |
| RPGQGLEWIGYINPSSGYTNYNQKFKDKATLTADKSSSTA | NYLTWYQQKPGQPPKLLIYWASTRESGVPDRFTGSG |
| YMQLSSLTSEDSAVYYCARIYYGNSFAYWGQGTTVTVSS | SGTDFTLTISSVQAEDLAVYYCQNDYSYPLTFGAGT |
| | KLELKR |
| QVQLQQSGPELVKPGALVKISCKASGYTFTSYDINWVKQR | QIVLTQSPAIMSASPGEKVTMTCSASSSISYMHWYQQ |
| PGQGLEWIGWIYPGDGSTKYNEKFKGKATLTADKSSSTAY | KPGTSPKRWIYDTSKLASGVPARFSGSGSGTSYSLTIS |
| MQLSSLTSENSAVYFCARGGYRYDEAMDYWGQGTTVTVS | SMEAEDAATYYCHQRSSYPYTFGGGTKLEIKR |
| S | |
| QIQLVQSGPELKKPGETVKISCKASGYTFTNYGMNWVKQA | DIVMTQSPSSLSVSAGEKVTMSCKSSQSLLNSGNQK |
| PGKGLKWMGWINTNTGEPTYAEEFKGRFAFSLETSASTAY | NYLAWYQQKPGQPPKLLIYGASTRESGVPDRFTGSG |
| LQINNLKNEDTATYFCARFSYGNSFAYWGQGTTVTVSS | SGTDFTLTISSVQAEDLAVYYCQNDHSYPLTFGAGT |
| | KLELKR |
| DVQLQESGPDLVKPSQSLSLTCTVTGYSITSGYNWHWIRQF | DIVMTQSPSSLTVTPGEKVTMTCKSSQSLFNSGNQK |
| PGNKMEWMGYIHYTGSTNYNPSLRSRISITRDTSKNQFFLQ | NYLTWYQQRPGQPPKMLIYWASTRESGVPDRFTGS |
| LNSVTTDDTATYYCTRIYNGNSFPYWGQGTSVTVSS | GSGTDFTLTISSVQAEDLAVFYCQNAYSFPYTFGGGT |
| | KLEIKR |
| QVQLQQSGAELARPGASVKMSCKASGYTFTSYTMHWVKQ | DIVMTQSPSSLTVTAGEKVTMSCKSSQSLLNSGNQK |
| RPGQGLEWIGYIDPSSGYTNYNQKFKDKATLTADKSSSTA | NYLTWYQQKPGQPPKLLIYWASTRESGVPDRFTGSG |
| YMQLSSLTSEDSAVYYCARIYYGNSFAYWGQGTTVTVSS | SGTDFTLTISSVQAEDLAVYYCQNDYSYPLTFGAGT |
| | KLELKR |
| QVQLQQSGAELARPGASVKMSCKASGYTFTSYTMHWVKQ | DIVMTQSPSSLTVTAGEKVTMSCKSSQSLLNSGNQK |
| RPGQGLEWIGYINPASGYTNYNQKFKDKATLTADKSSSTA | NYLTWYQQKPGQPPKLLIYWASTRESGVPDRFTGSG |
| YMQLSSLTSEDSAVYYCARIYYGNSFAYWGQGTTVTVSS | SGTDFTLTISSVQAEDLAVYYCQNDYSYPLTFGAGT |
| | KLELKR |
| QVQLQQSGAELARPGASVKMSCKASGYTFTSYTMHWVKQ | DIVMTQSPSSLTVTAGEKVTMSCKSSQSLLNSGNQK |
| RPGQGLEWIGYINPASGYTNYNQKFKDKATLTADKSSSTA | NYLTWYQQKPGQPPKLLIYWASTRESGVPDRFTGSG |
| YMQLSSLTSEDSAVYYCARIYYGNSFAYWGQGTTVTVSS | SGTDFTLTISSVQAEDLAVYYCQNDYSYPLTFGAGT |
| | KLELKR |
| QVQLVQSGAEVKKPGASVKVSCKASGYTFTSYTMHWVRQ | DIVMTQSPDSLAVSLGERATINCKSSQSLLNSGNQKN |
| APGQGLEWMGYINPASGYTNYNQKFKDRVTMTRDTSTST | YLTWYQQKPGQPPKLLIYWASTRESGVPDRFSGSGS |
| AYMELSSLRSEDTAVYYCARIYYGNSFAYWGQGTLVTVSS | GTDFTLTISSLQAEDVAVYYCQNDYSYPLTFGGGTK |
| | VEIKR |
| QVQLQESGPGLVKPSQTLSLTCTVSGGSISSGYNWHWIRQP | DIVMTQSPDSLAVSLGERATINCKSSQSLFNSGNQKN |
| PGKGLEWIGYIHYTGSTNYNPALRSRVTISVDTSKNQFSLK | YLTWYQQKPGQPPKLLIYWASTRESGVPDRFSGSGS |
| LSSVTAADTAVYYCARIYNGNSFPYWGQGTTVTVSS | GTDFTLTISSLQAEDVAVYYCQNAYSFPYTFGGGTK |
| | LEIKR |
| QVQLQESGPGLIKPSQTLSLTCTVSGGSISSGYNWHWIRQPP | DIVMTQSPDSLAVSLGERATINCKSSQSLFNSGNQKN |
| GKGLEWIGYIHYTGSTNYNPALRSRVTISVDTSKNQFSLKL | YLTWYQQKPGQPPKLLIYWASTRESGVPDRFSGSGS |
| SSVTAADTAIYYCARIYNGNSFPYWGQGTTVTVSS | GTDFTLTISSLQAEDVAVYYCQNAYSFPYTFGGGTK |
| | LEIKR. |

In a specific embodiment, the amino acid sequence of the antigen recognition unit that recognizes GPC3 is selected from or has 70-100% sequence identity with the sequence shown in the following table;

QVQLQESGGGLVQPGRSLRLSCAASGFTFSSYAMHWVRQAPGKGLEWVSA
ISGSGGSTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSTYAMTWVRQAPGKGLEWVSS
ISSSGESTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSTYAMAWVRQAPGKGLEWVSE
ISSSGSRTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSTYAMNWVRQAPGKGLEWVSA
ISMSGESTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSSYAMHWVRQAPGKGLEWVSA
ISSSGGSTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGHKFPVSWYQQYPGKAPKLLIYKNLLRPSGVPDRF
SGSKSGTSASLAITGLQAEDGADYYCQSYDSSLRVVFGGGTKVTVLG
QVQLQESGGGLVQPGRSLRLSCAASGFTFSSYAMHWVRQAPGKGLEWVSA
ISSSGGSTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGLMHNVSWYQQYPGKAPKLLIYKSSSRPSGVPDRF
SGSKSGTSASLAITGLQAEDGADYYCQSYDSSLRVVFGGGTKVTVLG
QVQLQESGGGLVQPGRSLRLSCAASGFTFSSYAMHWVRQAPGKGLEWVSA
ISSSGRSTYYADSVEGRFTISRDNSKNTLYLQMNSLRAEDTAVYYCAKDR
RGSHADALNVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
EVQLVQSGAEVKKPGASVKVSCKASGYTFSDYEMHWVRQAPGQGLEWMGA
IHPGSGDTAYNQRFKGRVTITADKSTSTAYMELSSLRSEDTAVYYCARFY
SYAYWGQGTLVTVSAGGGGSGGGGSGGGGSDIVMTQTPLSLPVTPGEPAS
ISCRSSQSLVHSNGNTYLQWYLQKPGQSPQLLIYKVSNRFSGVPDRFSGS
GSGTDFTLKISRVEAEDVGVYYCSQSIYVPYTFGQGTKLEIKR
QVQLQQSGTELVRPGASVKLSCKALGYTFTDYEMHWVKQTPVHGLEWIGA
IHPGSGDTAYNQRFKGKATLTADKSSSTAYMEYSSLTSEDSAVYYCTRFY
SYAYWGQGTLVTVSAGGGGSGGGGSGGGGSDVVMTQTPLSLPVSLGDQAS
ISCRSSQSLVHSNGNTYLQWYLQKPGQSPKLLIYKVSNRFSGVPDRFSGR
GSGTDFTLKISRVEAEDLGVYFCSQSIYVPYTFGGGTKLEIKR

or,

the amino acid sequence of the antigen recognition unit that recognizes claudin18.2 is selected from or has 70-100% sequence identity with the sequence shown in the following table;

QVQLQQSGAELARPGASVKMSCKASGYTFTSYTMHWVKQRPGQGLEWIGY
INPSSGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLQQSGPELVKPGALVKISCKASGYTFTSYDINWVKQRPGQGLEWIGW
IYPGDGSTKYNEKFKGKATLTADKSSSTAYMQLSSLTSENSAVYFCARGG
YRYDEAMDYWGQGTTVTVSSGGGGSGGGGSGGGGSQIVLTQSPAIMSASP
GEKVTMTCSASSSISYMHWYQQKPGTSPKRWIYDTSKLASGVPARFSGSG
SGTSYSLTISSMEAEDAATYYCHQRSSYPYTFGGGTKLEIKR
QIQLVQSGPELKKPGETVKISCKASGYTFTNYGMNWVKQAPGKGLKWMGW
INTNTGEPTYAEEFKGRFAFSLETSASTAYLQINNLKNEDTATYFCARFS
YGNSFAYWGQGTTVTVSSGGGGSGGGGSGGGGSDIVMTQSPSSLSVSAGE
KVTMSCKSSQSLLNSGNQKNYLAWYQQKPGQPPKLLIYGASTRESGVPDR
FTGSGSGTDFTLTISSVQAEDLAVYYCQNDHSYPLTFGAGTKLELKR
DVQLQESGPDLVKPSQSLSLTCTVTGYSITSGYNWHWIRQFPGNKMEWMG
YIHYTGSTNYNPSLRSRISITRDTSKNQFFLQLNSVTTDDTATYYCTRIY
NGNSFPYWGQGTSVTVSSGGGGSGGGGSGGGGSDIVMTQSPSSLTVTPGE
KVTMTCKSSQSLFNSGNQKNYLTWYQQRPGQPPKMLIYWASTRESGVPDR
FTGSGSGTDFTLTISSVQAEDLAVFYCQNAYSFPYTFGGGTKLEIKR
QVQLQQSGAELARPGASVKMSCKASGYTFTSYTMHWVKQRPGQGLEWIGY
IDPSSGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLQQSGAELARPGASVKMSCKASGYTFTSYTMHWVKQRPGQGLEWIGY
INPASGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLQQSGAELARPGASVKMSCKASGYTFTSYTMHWVKQRPGQGLEWIGY
INPASGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLVQSGAEVKKPGASVKVSCKASGYTFTSYTMHWVRQAPGQGLEWMGY
INPASGYTNYNQKFKDRVTMTRDTSTSTAYMELSSLRSEDTAVYYCARIY
YGNSFAYWGQGTLVTVSSGGGGSGGGGSGGGGSDIVMTQSPDSLAVSLGE
RATINCKSSQSLLNSGNQKNYLTWYQQKPGQPPKLLIYWASTRESGVPDR
FSGSGSGTDFTLTISSLQAEDVAVYYCQNDYSYPLTFGGGTKVEIKR
QVQLQESGPGLVKPSQTLSLTCTVSGGSISSGYNWHWIRQPPGKGLEWIG
YIHYTGSTNYNPALRSRVTISVDTSKNQFSLKLSSVTAADTAVYYCARIY
NGNSFPYWGQGTTVTVSSGGGGSGGGGSGGGGSDIVMTQSPDSLAVSLGE
RATINCKSSQSLFNSGNQKNYLTWYQQKPGQPPKLLIYWASTRESGVPDR
FSGSGSGTDFTLTISSLQAEDVAVYYCQNAYSFPYTFGGGTKLEIKR
QVQLQESGPGLIKPSQTLSLTCTVSGGSISSGYNWHWIRQPPGKGLEWIG
YIHYTGSTNYNPALRSRVTISVDTSKNQFSLKLSSVTAADTAIYYCARIY
NGNSFPYWGQGTTVTVSSGGGGSGGGGSGGGGSDIVMTQSPDSLAVSLGE
RATINCKSSQSLFNSGNQKNYLTWYQQKPGQPPKLLIYWASTRESGVPDR
FSGSGSGTDFTLTISSLQAEDVAVYYCQNAYSFPYTFGGGTKLEIKR.

In a preferred embodiment, the antigen recognition unit is connected to the extracellular domain of the TCR via a linker sequence.

In a preferred embodiment, the linker sequence includes (G₄S)ₙ, where n=1 to 4; or the encoding sequence of the linker has the nucleic acid sequence shown in SEQ ID NO: 5, 11 or 12.

In a preferred embodiment, the TCR subunit comprises an extracellular domain, and/or transmembrane domain, and/or intracellular domain; preferably, at least two of the extracellular domain, transmembrane domain and intracellular domain are from the same TCR subunit.

In a preferred embodiment, the TCR subunit comprises a TCR intracellular domain containing a stimulatory domain, and the stimulatory domain is selected from the intracellular signaling domain of CD3ε, CD3γ, or CD3δ, or an amino acid sequence thereof having at least one modification.

In a preferred embodiment,

(i) a light chain variable region of the amino acid sequence of the antigen recognition unit that binds to GPC3, wherein the light chain variable region comprises an amino acid sequence having at least one but not more than 30 modifications as compared with amino acid sequence of the light chain variable region of the anti-GPC3 antibody provided herein, or a sequence having 90-99% identity with the amino acid sequence of the light chain variable region of the anti-GPC3 antibody provided herein; or

(ii) a light chain variable region of the amino acid sequence of the antigen recognition unit that binds to claudin18.2, wherein the light chain variable region comprises an amino acid sequence having at least one but not more than 30 modifications as compared with amino acid sequence of the light chain variable region of the anti-claudin18.2 antibody provided herein, or a sequence having 90-99% identity with the amino acid sequence of the light chain variable region of the anti-claudin18.2 antibody provided herein.

In a preferred example,

(i) a heavy chain variable region of the amino acid sequence of the antigen recognition unit that binds to GPC3, wherein the heavy chain variable region comprises an amino acid sequence having at least one but not more than 30 modifications as compared with amino acid sequence of the heavy chain variable region of the anti-GPC3 antibody provided herein, or a sequence having 90-99% identity with the amino acid sequence of the heavy chain variable region of the anti-GPC3 antibody provided herein; or

(ii) a heavy chain variable region of the amino acid sequence of the antigen recognition unit that binds to claudin18.2, wherein the heavy chain variable region comprises an amino acid sequence having at least one but not more than 30 modifications as compared with amino acid sequence of the heavy chain variable region of the anti-claudin18.2 antibody provided herein, or a sequence having 90-99% identity with the amino acid sequence of the heavy chain variable region of the anti-claudin18.2 antibody provided herein.

In a preferred example, the TFP comprises the extracellular domain of the TCR subunit, and the extracellular domain includes the extracellular domain of a protein or a part thereof selected from the group consisting of: TCRα chain, TCRβ chain, CD3εTCR subunit, CD3γTCR subunit, CD3δTCR subunit, functional fragments thereof, and an amino acid sequence thereof with at least one but no more than 20 modifications.

In a preferred example, the TFP includes a transmembrane domain, and the transmembrane domain includes a transmembrane domain of a protein selected from the group consisting of: TCRα chain, TCRβ chain, CD3εTCR subunit, CD3γTCR subunit, The CD3δTCR subunit, functional fragments thereof, and an amino acid sequence thereof with at least one but no more than 20 modifications.

In a preferred example, the TFP includes a transmembrane domain, and the transmembrane domain includes a transmembrane domain of a protein selected from the group consisting of: TCRα chain, TCRβ chain, TCRζ chain, CD3εTCR subunit, CD3γTCR subunits, CD3δTCR subunits, CD45, CD4, CD5, CD8, CD9, CD16, CD22, CD33, CD28, CD37, CD64, CD80, CD86, CD134, CD137, CD154, functional fragments thereof, and an amino acid sequence thereof with at least one but not more than 20 modifications.

In a preferred example, the antigen recognition unit includes an antibody or a fragment thereof. Preferably, the antibody fragment is Fab, Fab′, F(ab′)2, Fv fragment, scFv, sdFv, Fd fragment composed of VH and CH1 domains, linear antibody, single domain antibody, or camelid VHH domain, and more preferably, the antibody is a scFv.

In a preferred example, the TFP further includes a costimulatory domain.

In a preferred example, the costimulatory domain is a functional signaling domain obtained from a protein selected from the group consisting of: OX40, CD2, CD27, CD28, CDS, ICAM-1, LFA-1 (CD11a/CD18), ICOS (CD278) and 4-1BB (CD137), and an amino acid sequence thereof with at least one but no more than 20 modifications.

In a second aspect, the present invention provides a combination of the fusion protein and other factors, the other factors including cytokines, transcription factors, chemokines, and/or combinations thereof, and the expression cassette is constitutively or inducibly expressed; preferably, the promoter of the expression cassette is an immune cell inducible promoter; preferably, the immune cell inducible promoter is NFAT6 promoter; and preferably, the NFAT6 promoter is reversely regulated.

In a preferred example, the fusion protein and other factors are expressed by the same nucleic acid molecule, or by different nucleic acid molecules.

In a preferred example, the fusion protein and other factors are expressed by the same nucleic acid molecule, and the expression cassettes of the other factors and the expression cassettes of TFP, and the expression cassettes of the other factors are directly connected or connected by tandem fragments, and the tandem fragments are selected from F2A, PA2, T2A, and/or E2A.

In a preferred example, the cytokine is selected from IL-7 or IL-12, the chemokine is CCL19 or CCL21; the transcription factor is RUNX3; preferably, the other factors are a combination of cytokines and chemokines; and preferably, the other factors are a combination of IL7 and CCL21, or a combination of IL7 and CCL19.

In a preferred example, the at least one but no more than 20 modifications include amino acid modifications that mediate cell signal transduction, or amino acid modifications that are phosphorylated in response to the binding of a ligand to TFP.

In a preferred example, the TFP includes the immunoreceptor tyrosine activation motif (ITAM) of the TCR subunit, and the ITAM includes the ITAM or a part thereof of a protein selected from the group consisting of: CD3ζTCR subunit, CD3εTCR subunit, CD3γTCR subunit, CD3δTCR subunit, TCRζ chain, Fcε receptor 1 chain, FCε receptor 2 chain, Fcγ receptor 1 chain, Fcγ receptor 2a chain, Fcγ receptor 2b1 chain, Fcγ receptor 2b2 chain, Fcγ receptor 3a chain, Fcγ receptor 3b chain, Fcβ receptor 1 chain, TYROBP (DAP12), CD5, CD16a, CD16b, CD22, CD23, CD32, CD64, CD79a, CD79b, CD89, CD278, CD66d, and functional fragments thereof, and an amino acid sequence thereof with at least one but no more than 20 modifications.

In a preferred example, the ITAM replaces the ITAM of CD3γ, CD3δ or CD3ε.

In a preferred example, the ITAM is selected from CD3ζTCR subunit, CD3εTCR subunit, CD3γTCR subunit, or CD3δTCR subunit, and replaces different ITAMs selected from CD3ζTCR subunit, CD3εTCR subunit, CD3γTCR subunit or CD3δTCR subunit.

In a preferred example, the TFP molecule further includes a leader sequence.

In a third aspect, the present invention provides a nucleic acid molecule expressing the fusion protein described in the first aspect or the combination described in the second aspect.

In a preferred example, the nucleic acid is composed of DNA and/or RNA.

In a preferred example, the nucleic acid is mRNA.

In a preferred example, the nucleic acid comprises nucleotide analogs.

In the fourth aspect, the present invention provides a vector comprising the nucleic acid molecule described in the third aspect.

In a preferred example, the vector is selected from the group consisting of DNA, RNA, plasmid, lentiviral vector, adenoviral vector, Rous sarcoma virus (RSV) vector and retroviral vector.

In the fifth aspect, the present invention provides a cell comprising the vector of the fourth aspect or having the nucleic acid molecule of the third aspect integrated into its genome.

In a preferred example, the cells are human T cells, preferably allogeneic T cells.

In a sixth aspect, the present invention provides a protein complex comprising:

i) the fusion protein TFP molecule of the first aspect; and

ii) at least one endogenous TCR subunit or endogenous TCR complex.

In a seventh aspect, the present invention provides a method for preparing cells, comprising transducing T cells with the vector of the fourth aspect or the nucleic acid molecule of the third aspect.

In an eighth aspect, the present invention provides a method for producing an RNA-engineered cell population, comprising introducing in vitro transcribed RNA or synthetic RNA into the cell,

(i) wherein the RNA includes the nucleic acid of the third aspect.

In a ninth aspect, the present invention provides a method for providing anti-tumor immunities in a mammal, comprising administering to the mammal an effective amount of the fusion protein of the first aspect and the combination of the second aspect, the nucleic acid molecule of the third aspect, the vector of the fourth aspect, or the cell of the fifth aspect.

In a preferred example, the mammal is a human.

In a tenth aspect, the present invention provides a method for treating a mammal suffering from a disease related to the expression of GPC3 or claudin 18.2, comprising administering to the mammal an effective amount of the fusion protein of the first aspect, the combination of the second aspect, the nucleic acid molecule of the third aspect, the vector of the fourth aspect, or the cell of the fifth aspect.

In a preferred example, the disease related to the expression of GPC3 or claudin 18.2 is selected from colon cancer, rectal cancer, renal cell carcinoma, liver cancer, lung cancer, small intestine cancer, esophageal cancer, melanoma, bone cancer, pancreatic cancer, skin cancer, head and neck cancer, skin or intraocular malignant melanoma, uterine cancer, ovarian cancer, rectal cancer, anal cancer, stomach cancer, testicular cancer, uterine cancer, fallopian tube cancer, endometrial cancer, cervical cancer, vaginal cancer, endocrine system cancer, thyroid cancer, parathyroid cancer, adrenal cancer, soft tissue sarcoma, urethral cancer, penile cancer, bladder cancer, kidney or ureter cancer, renal pelvis cancer, central nervous system (CNS) tumors, tumor angiogenesis, spinal tumors, brainstem glioma, pituitary adenoma, Kaposi's sarcoma, epidermoid carcinoma, squamous cell carcinoma, non-cancer related indications related to the expression of GPC3 or claudin18.2; and preferably, selected from liver cancer, lung cancer, breast cancer, ovarian cancer, kidney cancer, thyroid cancer, stomach cancer, colorectal cancer, pancreatic cancer, esophageal cancer.

In a preferred example, the cells expressing TFP molecules are administered in combination with an agent that increases the efficacy of the cells expressing TFP molecules.

In a preferred example, compared with a mammal that is administered with an effective amount of T cells expressing GPC3 chimeric antigen receptor (CAR) or claudin18.2 chimeric antigen receptor (CAR), there are fewer cytokines released in the mammal.

In a preferred example, the cells expressing TFP molecules are administered in combination with an agent that improves one or more side effects associated with the administration of cells expressing TFP molecules.

In a preferred example, the cell expressing the TFP molecule is administered in combination with an agent for treating the disease related to GPC3 or claudin 18.2.

In the eleventh aspect, the present invention provides the fusion protein of the first aspect, the combination of the second aspect, the nucleic acid molecule of the third aspect, the vector of the fourth aspect, or the cell of the fifth aspect as a medicament.

In a preferred example, the medicament is a medicament for preventing or treating a disease related to the expression of GPC3 or claudin 18.2.

In a preferred example, the disease related to the expression of GPC3 or claudin 18.2 is selected from colon cancer, rectal cancer, renal cell carcinoma, liver cancer, lung cancer, small intestine cancer, esophageal cancer, melanoma, bone cancer, pancreatic cancer, skin cancer, head and neck cancer, skin or intraocular malignant melanoma, uterine cancer, ovarian cancer, rectal cancer, anal cancer, stomach cancer, testicular cancer, uterine cancer, fallopian tube cancer, endometrial cancer, cervical cancer, vaginal cancer, endocrine system cancer, thyroid cancer, parathyroid cancer, adrenal cancer, soft tissue sarcoma, urethral cancer, penile cancer, bladder cancer, kidney or ureter cancer, renal pelvis cancer, central nervous system (CNS) tumors, tumor angiogenesis, spinal tumors, Brainstem glioma, pituitary adenoma, Kaposi's sarcoma, epidermoid carcinoma, squamous cell carcinoma, non-cancer related indications related to GPC3 or claudin18.2 expression; and preferably, selected from liver cancer, lung cancer, breast cancer, ovarian cancer, kidney cancer, thyroid cancer, stomach cancer, colorectal cancer, pancreatic cancer, esophageal cancer.

In one aspect of the present invention, a TCR fusion protein is provided. The TCR fusion protein comprises a tumor antigen recognition unit and a TCR unit, the tumor antigen recognition unit is an antibody that recognizes a solid tumor antigen, and the TCR unit contains at least a portion of CD3 extracellular domain, CD3 transmembrane domain and CD3 intracellular signal domain.

In a preferred embodiment, the solid tumor antigen is selected from GPC3, claudin 6, EGFR, EGFRvIII, claudin 18.2. More preferably, the solid tumor antigen is GPC3 or claudin 18.2.

In a preferred embodiment, the solid tumor is selected from the group consisting of colon cancer, rectal cancer, renal cell carcinoma, liver cancer, lung cancer, small bowel cancer, esophageal cancer, melanoma, bone cancer, pancreatic cancer, skin cancer, head and neck cancer, skin or intraocular melanoma, uterine cancer, ovarian cancer, rectal cancer, anal cancer, stomach cancer, testicular cancer, uterine cancer, fallopian tube cancer, endometrial cancer, cervical cancer, vagina cancer, vaginal cancer, endocrine system cancer, thyroid cancer, parathyroid cancer, adrenal cancer, soft tissue sarcoma, urethral cancer, penile cancer, bladder cancer, kidney or ureter cancer, renal pelvis cancer, central nervous system (CNS) tumor, tumor angiogenesis, spine tumor, brainstem glioma, pituitary adenoma, Kaposi's sarcoma, epidermoid carcinoma, squamous cell carcinoma; and preferably, selected from liver cancer, lung cancer, breast cancer, ovarian cancer, kidney cancer, thyroid cancer, stomach cancer, colorectal cancer, stomach cancer, pancreatic cancer, esophageal cancer.

In a preferred embodiment, the antibody is a complete immunoglobulin or antibody fragment; preferably, the antibody fragment is Fab, Fab′, F(ab′)2, Fv fragment, scFv, sdFv, Fd fragments composed of VH and CH1 domains, linear antibodies, single domain antibodies, or camelid VHH domains; and more preferably, the antibody is a scFv.

In a preferred embodiment, the antibody has HCDR1 shown in SEQ ID NO: 15, HCDR2 shown in SEQ ID NO: 16, HCDR3 shown in SEQ ID NO: 17, and LCDR1 SEQ ID NO: 18, LCDR2 shown in SEQ ID NO: 19, and LCDR3 shown in SEQ ID NO: 20;

In a preferred embodiment, the antibody has the VH shown in SEQ ID NO: 21 and the VL shown in SEQ ID NO: 22.

In a preferred embodiment, the CD3 intracellular signal domain is the intracellular signal domain of CD3ε, CD3γ, CD3ζ, or CD3δ, preferably, the intracellular signal domain of CD3ε and CD3γ, and more preferably, the intracellular signal domain of CD3ε.

In a preferred embodiment, the tumor antigen recognition unit is operably connected to the TCR unit.

In a preferred embodiment, the tumor antigen recognition unit is connected to the extracellular domain of the TCR via a linker.

Preferably, the linker sequence is (G₄S)ₙ, n=any natural number between 1 and 4, or the linker has the nucleic acid sequence shown in SEQ ID NO: 5, 11 or 12.

In a preferred embodiment, the TCR unit contains at least a part of the TCR extracellular domain, TCR transmembrane domain and CD3 intracellular signal domain from the same TCR subunit.

In a preferred embodiment, the TCR unit has the amino acid sequence shown in SEQ ID NO: 3 or 8.

In a preferred embodiment, the nucleic acid encoding the TCR fusion protein has the sequence shown in SEQ ID NO: 7, 8, 13, 14.

In one aspect of the present invention, a nucleic acid encoding the TCR fusion protein of the present invention is provided.

In one aspect of the present invention, an expression vector is provided, which contains the nucleic acid encoding the TCR fusion protein of the present invention.

In one aspect of the present invention, a virus is provided, which comprises the expression vector of the present invention.

In one aspect of the present invention, T cells expressing the TCR fusion protein of the present invention are provided.

In a preferred embodiment, the T cell also expresses an cytokine, transcription factor, another chimeric receptor, chemokine, chemokine receptor, siRNA reducing PD-1 expression, or a protein blocking PD-L1, TCR, or safety switch.

In another preferred embodiment, the factor is a fusion protein expressing a cytokine.

In another preferred embodiment, the cytokine is selected from IL-12, IL-18, IL-21, or type I interferon.

In another preferred embodiment, the fusion protein contains cytokines and chemokines.

In another preferred embodiment, the chemokine is CCL19 or CCL21; preferably, the fusion protein contains IL7 and CCL21, or IL7 and CCL19.

In another preferred embodiment, the chemokine receptor includes CCR2, CCR5, CXCR2, or CXCR4;

In another preferred embodiment, the safety switch includes iCaspase-9, Truncated EGFR or RQR8.

In another preferred embodiment, the other chimeric receptor is selected from the group consisting of chimeric antigen receptor (CAR), modified T cell (antigen) receptor (TCR), T cell fusion protein (TFP), T Cell Antigen Coupler (TAC).

In another preferred embodiment, the transcription factor is RUNX3.

In another preferred embodiment, the T cells also express a fusion protein inhibiting the tumor microenvironment or a fusion protein stimulating T cells.

In another preferred embodiment, the chemokine is a lymphocyte chemokine, and preferably, the chemokine is CCL21.

In one aspect of the present invention, there is provided the use of the expression vector of the present invention, or the virus of the present invention, or the cell of the present invention for the preparation of a medicament for inhibiting tumors.

It should be understood that within the scope of the present invention, the above-mentioned technical features of the present invention and the technical features specifically described in the following (such as the embodiments) can be combined with each other to form a new or preferred technical solution, which will not be repeated herein one by one.

### DESCRIPTION OF DRAWINGS

FIG. 1A shows the constructed TCR fusion protein vector targeting Claudin 18.2 and GPC3; and FIG. 1B shows the result of the positive rate of T cells expressing the TCR fusion protein;

FIG. 2 shows the in vitro killing results of T cells expressing TCR fusion protein on pancreatic cancer cells and gastric cancer cells;

FIG. 3 shows the detection results of cytokine secretion after co-incubation of T cells expressing TCR fusion protein with pancreatic cancer cells and gastric cancer cells;

FIG. 4 shows the detection results of the positive rate of different TCR fusion proteins;

FIG. 5 shows the cell killing effects of T cells expressing TCR fusion protein targeting GPC3;

FIG. 6 shows the secretion of different cytokines in T cells expressing TCR fusion protein detected by ELISA;

FIG. 7 shows the in vivo anti-tumor results of T cells expressing TCR fusion protein targeting GPC3;

FIG. 8 shows the body weight of mice in each group after treated with T cells expressing TCR fusion protein targeting GPC3.

### MODES FOR CARRYING OUT THE INVENTION

The present application provides a technical solution for treating a solid tumor, especially for treating gastric cancer and pancreatic cancer by using cells expressing TCR fusion protein.

Unless specifically defined, all technical and scientific terms used herein have the same meanings commonly understood by those skilled in the fields of gene therapy, biochemistry, genetics, and molecular biology. All methods and materials similar or equivalent to those described herein can be used in the practice or testing of the present invention, among which suitable methods and materials are described herein. All publications, patent applications, patents and other references mentioned in this article are incorporated herein by reference in their entirety. In case of conflict, the specification, including definitions, will control. In addition, unless otherwise specified, the materials, methods, and examples are illustrative only and not intended to be limiting.

Unless otherwise specified, the practice of the present invention will use traditional techniques of cell biology, cell culture, molecular biology, transgenic biology, microbiology, recombinant DNA and immunology, all of which fall within the technical scope of the art. These techniques are fully explained in the literature. See, for example, Current Protocols in Molecular Biology (Frederick M. AUSUBEL, 2000, Wileyand son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gaited., 1984); Mullis et al. U.S. Pat. No. 4,683,195; Nucleic Acid Hybridization (B. D. Harries & S. J. Higginseds. 1984); Transcription And Translation (B. D. Hames & S. J. Higginseds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson [symbol] M. Simon, eds.-in-chief, Academic Press, Inc., New York), especially Vols. 154 and 155 (Wu et al. eds.) and Vol. 185, “Gene Expression Technology” (D. Goeddel, ed.); Gene Transfer Vectors For Mammalian Cells (J. H. Miller [symbol] M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Vol I-IV (D. M. Weir and C. C. Blackwell, eds., 1986); and Manipulating the Mouse Embryo (Cold Spring Harbor Laboratory Press, Cold Spring Harbor, N.Y., 1986).

To facilitate a better understanding of the present invention, relevant terms are defined as follows:

As used herein, “about” may mean, depending on the specific circumstances and known by a skilled person in the art, plus or minus less than 1% or 1%, 2%, 3%, 4%, 5%, 6%, 7%, 8%, 9%, 10%, 11%, 12%, 13%, 14%, 15%, 16%, 17%, 18%, 19%, 20%, 25%, 30% or more than 30%.

The term “T cell (antigen) receptor (TCR), also known as TCR subunit, or TCR unit” is a characteristic mark on the surface of all T cells, which binds to CD3 by a non-covalent bond to form TCR-CD3 complex. TCR is responsible for recognizing antigens bound to major histocompatibility complex molecules. TCR is a heterodimer composed of two different peptide chains, composed of two peptide chains, α and β. Each peptide chain can be divided into several parts, such as variable region (V region), constant region (C region), transmembrane region and cytoplasmic region, characterized in that the cytoplasmic region is very short. TCR molecules belong to the immunoglobulin superfamily, and their antigen specificity exists in the V region; each of V regions (Vα, Vβ) has three hypervariable regions CDR1, CDR2, and CDR3, in which CDR3 has the largest variation and directly determines the binding specificity of TCR to an antigen. When TCR recognizes the MHC-antigen peptide complex, CDR1 and CDR2 recognize and bind to the side wall of the antigen binding groove of the MHC molecule, and CDR3 directly binds to the antigen peptide. TCR is divided into two categories: TCR1 and TCR2; TCR1 is composed of two chains, γ and δ, and TCR2 is composed of two chains, α and β. The recognition ability of these natural (or manufactured by other means) “anti-cancer” T cells is generally weak, therefore they cannot favorably attack cancer cells. In this case, a partial genetic modification method can be used to improve the “affinity” and effectiveness of these TCRs to the corresponding TAA, that is, high-affinity TCR. “Genetically modified TCR” technology is therefore called “affinity-enhanced TCR” technology. The gene modified T cell receptor uses the constant region domains of the heavy and light chains of antibodies that belong to the same immunoglobulin superfamily with the TCR molecule to replace the constant region domains of the B chain and a chain, respectively, to form a chimeric TCR molecule (chim-TCR).

The term “TCR fusion protein” or “TFP” includes recombinant proteins derived from various TCR proteins, which are generally capable of: i) binding to the surface antigen on target cells; ii) interacting with other polypeptide components of an intact TCR complex when localized to T cells. A “TFP T cell” is a T cell that has been transduced with (for example, according to the methods disclosed herein) and expresses TFP, for example, a T cell introduced with a natural TCR. In some embodiments, the T cell is a CD4+ T cell, CD8+ T cell, or CD4+/CD8+ T cell. In some embodiments, the TFP T cell is a NK cell. In some embodiments, the TFP T cell is a γδ T cell.

The “TCR fusion protein” of the present invention includes an extracellular antigen-binding domain (also called an antigen recognition unit), TCR transmembrane domain, and intracellular domain. The antigen-binding domain is a continuous polypeptide chain, including, for example, a single domain antibody fragment (sdAb), a part of single-chain antibody (scFv) derived from murine, humanized or human antibodies (Harlow et al., 1999, Using Antibodies: A Laboratory Manual, Cold Spring Harbor Laboratory Press, NY; Harlow et al. Human, 1989, Antibodies: A Laboratory Manual, Cold Spring Harbor, N.Y.; Houston et al., 1988, Proc. Natl. Acad. Sci. USA 85: 5879-5883; Bird et al., 1988, Science 242: 423-426). In one aspect, the antigen binding domain of the TFP composition of the invention includes an antibody fragment. In another aspect, TFP includes an antibody fragment containing scFv or sdAb.

The present invention includes a recombinant DNA construct encoding TFP, wherein the TFP comprises an antibody fragment specifically binding to GPC3 or Claudin 18.2, and the sequence of the antibody fragment is adjacent to the nucleic acid sequence encoding the TCR unit or a part thereof and in the same reading frame. The TFP provided herein can associate with one or more endogenous (alternatively, one or more exogenous, or endogenous and exogenous combinations) TCR units to form a functional TCR complex.

In one aspect, the TFP of the present invention comprises a target-specific binding element, which is also referred to as an antigen recognition unit. The choice of a part depends on the type and number of target antigens that define the surface of the target cell. For example, the antigen recognition unit can be selected to recognize the target antigen as a cell surface marker associated with a specific disease state on the target cell. Therefore, examples of cell surface markers that can be used as target antigens of the antigen recognition unit in the TFP of the present invention include markers related to viral, bacterial and parasitic infections, autoimmune diseases, and cancerous diseases (e.g., malignant diseases).

The extracellular domain of TFP of the present invention can be derived from natural sources or from recombinant sources. In the case of natural sources, the domain can be derived from any protein, especially a membrane-bound or transmembrane protein. In one aspect, the extracellular domain can associate with the transmembrane domain. Extracellular domains particularly useful in the present invention may include at least the following extracellular regions: for example, the α, β, or ζ chains of the T cell receptor, or CD3ε, CD3γ, or CD3δ, or in alternative embodiments, include CD28, CD45, CD4, CD5, CD8, CD9, CD16, CD22, CD33, CD37, CD64, CD80, CD86, CD134, CD137, CD154.

The transmembrane domain of the TFP of the present invention can be derived from natural sources or recombinant sources. In the case of natural sources, the domain can be derived from any membrane-bound or transmembrane protein. In one aspect, the transmembrane domain can signal to the intracellular domain when TFP binds to a target. Transmembrane domains particularly useful in the present invention may include at least the following transmembrane regions: for example, the α, β, or ζ chains of T cell receptors, or CD28, CD3ε, CD45, CD4, CD5, CD8, CD9, CD16, CD22, CD33, CD37, CD64, CD80, CD86, CD134, CD137, CD154. In some cases, the transmembrane domain may be connected to the extracellular region of TFP (such as the antigen binding domain of TFP) via a hinge (such as a hinge from a human protein). For example, in one embodiment, the hinge may be a human immunoglobulin (Ig) hinge, such as an IgG4 hinge or CD8a hinge.

The linker of the present invention is optionally a short oligopeptide of 2 to 10 amino acids in length, or a polypeptide linker can form a connection between the transmembrane domain and the cytoplasmic region of TFP. The glycine-serine doublet provides a particularly suitable linker.

If TFP contains CD3γ, δ, or ε polypeptides, the cytoplasmic domain of TFP may include intracellular signaling domains; and TCRα and TCRβ subunits usually lack signaling domains. The intracellular signaling domain is generally responsible for activating at least one normal effector function of immune cells into which TFP has been introduced. The term “effector function” refers to the specialized function of a cell. For example, the effector function of T cells can be cytolytic activity or auxiliary activity, including the secretion of cytokines. In one example, after co-incubated with tumor cells overexpressing TFP targeting antigens, the TFP-T cells can secrete large amounts of IFN-γ, Granzyme-B, IL2, TNF-α and GM-CSF; and compared with CAR T cells constructed with the same antigen recognition unit, TCR-T maintains the same significant cytotoxicity, while the cytokine secretion is significantly reduced, thereby effectively reducing the possibility of cytokine storm.

Therefore, the term “intracellular signaling domain” refers to a part of a protein that transduces effector function signals and guides cells to perform specialized functions. Although the entire intracellular signaling domain can usually be used, in many cases it is not necessary to use the entire chain. If the truncated part of the intracellular signaling domain is used, such a truncated part can be used instead of the complete chain as long as it transduces the effector function signal. Therefore, the term “intracellular signaling domain” is intended to include any truncated portion of the intracellular signaling domain sufficient to transduce effector function signals.

Examples of intracellular signaling domains used in TFPs of the present invention include cytoplasmic sequences of T cell receptors (TCRs) and co-receptors that act synergistically to initiate signal transduction after antigen receptor engagement, and derivatives or variants of any of these sequences and any recombinant sequence with the same functional capabilities. T cell activation can be mediated by two different types of cytoplasmic signaling sequences: the signaling sequence (primary intracellular signal transduction domain) that initiates antigen-dependent primary activation through TCR and the signaling sequence acts in an antigen-independent manner to provide secondary or costimulatory signals (secondary cytoplasmic domains, such as costimulatory domains). The primary signaling domain modulates the primary activation of the TCR complex in a stimulating manner or in an inhibitory manner. Primary intracellular signaling domains that act in a stimulating manner may contain signaling motifs, which are called immunoreceptor tyrosine-based activation motifs (ITAM).

Examples of ITAM-containing primary intracellular signaling domains that are particularly useful in the present invention include intracellular signaling domains of CD3ζ, FcRγ, FcRβ, CD3γ, CD3δ, CD3ε, CD5, CD22, CD79a, CD79b, and CD66d. In one embodiment, the TFP of the invention comprises an intracellular signaling domain, such as the primary signaling domain of CD3ε. In one embodiment, the primary signaling domain comprises a modified ITAM domain, such as a mutant ITAM domain having altered (e.g., increased or decreased) activity compared with the natural ITAM domain. In one embodiment, the primary signaling domain includes a primary intracellular signaling domain containing a modified ITAM, for example, a primary intracellular signaling domain containing an optimized and/or truncated ITAM. In one embodiment, the primary signaling domain comprises one, two, three, four or more ITAM motifs.

The intracellular signaling domain of TFP may comprise the CD3ζ signaling domain alone, or it may be combined with any other desired intracellular signaling domain useful in the TFP of the present invention. For example, the intracellular signaling domain of TFP may include a CD3ε chain portion and a co-stimulatory signaling domain. The costimulatory signaling domain refers to a part of TFP that contains the intracellular domain of a costimulatory molecule. Co-stimulatory molecules are cell surface molecules other than antigen receptors or ligands thereof, which are required for effective response of lymphocytes to antigens. Examples of such molecules include CD27, CD28, 4-1BB (CD137), OX40, DAP10, DAP12, CD30, CD40, PD1, ICOS, lymphocyte function associated antigen-1 (LFA-1), CD2, CD7, LIGHT, NKG2C, B7-H3 and ligands that specifically bind to CD83, etc.

The intracellular signaling sequences in the cytoplasmic portion of the TFP of the present invention can be connected to each other in a random or specified order. Optionally, a short oligopeptide or polypeptide linker, for example, 2 to 10 amino acids in length (for example, 2, 3, 4, 5, 6, 7, 8, 9 or 10 amino acids), can form a connection between the intracellular signaling sequences.

In another aspect, the TFP-expressing cells described herein can further express another factor, such as cytokines, transcription factors, chemokines, and/or combinations thereof, to increase T cell proliferation, cell survival, anti-apoptosis effect, tumor infiltration and other functions to improve anti-tumor activity. In one embodiment, T cells expressing the TFP also express cytokine IL-7 and chemokine CCL21, cytokine IL-12 or transcription factor RUNX3, and when co-incubated with tumor cells expressing TFP targeting antigens, the TFP-T cells can significantly increase the in vitro killing toxicity to the tumor cells, and significantly inhibit the formation of the tumor cells in vivo subcutaneously xenograft tumors; and when the in vitro cytokine secretion was tested, it is found that the TFP-T cells can secrete a large amount of IFN-γ and Granzyme-B, in which TFP-T cells expressing IL-12 exhibit the largest secretion.

The present invention also includes RNA constructs encoding TFP that can be directly transfected into cells. The method for producing mRNA for transfection can involve in vitro transcription (IVT) of the template with specially designed primers, and then adding poly A to produce a construct containing 3′ and 5′ untranslated sequences (“UTR”), 5′ cap and/or the internal ribosome entry site (IRES), the nucleic acid to be expressed and poly-A tail, usually 50-2000 bases in length. The RNA thus produced can effectively transfect different types of cells. In one aspect, the template contains the sequence of TFP. In one aspect, the anti-mesothelin TFP is encoded by messenger RNA (mRNA). In one aspect, mRNA encoding TFP against GPC3 or Claudin 18.2 is introduced into T cells to generate TFP-T cells. In one embodiment, in vitro transcribed RNA TFP can be introduced into cells by transient transfection. RNA is produced by in vitro transcription using a template generated by polymerase chain reaction (PCR). Appropriate primers and RNA polymerase can be used to directly convert DNA of interest from any source into a template for in vitro mRNA synthesis by PCR. The source of DNA can be, for example, genomic DNA, plasmid DNA, phage DNA, cDNA, synthetic DNA sequence or any other suitable DNA source. The required template for in vitro transcription is the TFP of the present invention. In one embodiment, the DNA to be used for PCR contains an open reading frame. The DNA may be derived from a naturally occurring DNA sequence in the genome of an organism. In one embodiment, the nucleic acid may comprise some or all of the 5′ and/or 3′ untranslated region (UTR). The nucleic acid may include exons and introns. In one embodiment, the DNA to be used for PCR is a human nucleic acid sequence. In another embodiment, the DNA to be used for PCR is a human nucleic acid sequence comprising 5′ and 3′ UTR. Alternatively, the DNA may be an artificial DNA sequence that is not normally expressed in a naturally occurring organism. An exemplary artificial DNA sequence is a sequence that contains portions of a gene that are joined together to form an open reading frame encoding a fusion protein. The DNA parts that are linked together can be from a single organism or from more than one organism.

PCR is used to generate a template for in vitro transcription of mRNA, which is used for transfection. Methods of performing PCR are well known in the art. The primer used for PCR is designed to have a region that is substantially complementary to the DNA region to be used as a PCR template. As used herein, “substantially complementary” refers to a nucleotide sequence in which most or all of the bases in the primer sequence are complementary, or one or more bases are non-complementary or mismatched. The primers that can be used for PCR can be produced by synthetic methods known in the art.

The invention also provides nucleic acid molecules encoding one or more of the TFP constructs described herein. The invention also provides a vector into which the DNA of the invention is inserted. Vectors derived from retroviruses such as lentiviruses are suitable tools to achieve long-term gene transfer since they allow long-term, stable integration of transgenes and their propagation in daughter cells. For example, nucleic acids can be cloned into vectors including but not limited to plasmids, phagemids, phage derivatives, animal viruses, and cosmids. Viruses that can be used as vectors include, but are not limited to, retrovirus, adenovirus, adeno-associated virus, herpes virus, and lentivirus. The present invention is not limited to the use of constitutive promoters, while inducible promoters are also considered. The use of an inducible promoter provides a molecular switch that can initiate the expression of an operably linked polynucleotide sequence when expression is required, or close the expression when expression is not required. Examples of inducible promoters include, but are not limited to, NFAT6 promoter, metallothionein promoter, glucocorticoid promoter, progesterone promoter, and tetracycline-regulated promoter.

Biological methods for introducing polynucleotides of interest into host cells include the use of DNA and RNA vectors. Viral vectors, especially retroviral vectors, have become the most widely used method for inserting genes into mammalian, such as human cells. Other viral vectors can be derived from lentivirus, poxvirus, herpes simplex virus I, adenovirus and adeno-associated virus.

Before expansion and genetic modification, a source of T cells is obtained from a subject. Examples of subjects include humans, dogs, cats, mice, rats, and transgenic species thereof. T cells can be obtained from many sources, including peripheral blood mononuclear cells, bone marrow, lymph node tissue, umbilical cord blood, thymus tissue, tissue from the site of infection, ascites, pleural effusion, spleen tissue, and tumors. In certain aspects of the invention, any number of T cell lines available in the art can be used. In certain aspects of the present invention, any number of techniques known to a skilled person, such as Ficoll™ separation technology, can be used to obtain T cells from blood units collected from a subject. In a preferred aspect, cells from the circulating blood of an individual are obtained by apheresis. Products obtained by apheresis blood apheresis usually contain lymphocytes, including T cells, monocytes, granulocytes, B cells, other nucleated white blood cells, red blood cells and platelets. In one aspect, the cells collected by apheresis can be washed to remove the plasma fraction and placed in an appropriate buffer or medium for subsequent processing steps. Multiple rounds of selection can also be used in the context of the invention. In some aspects, it may be necessary to perform a selection procedure and use “unselected” cells during activation and expansion. “Unselected” cells can also undergo other rounds of selection.

Generally, the T cell of the present invention can be expanded by contacting with a surface to which an agent that stimulates the CD3/TCR complex-related signal and a ligand that stimulates a costimulatory molecule on the surface of the T cell are attached. In particular, the T cell population can be stimulated as described herein, for example by contacting with an anti-CD3 antibody or antigen-binding fragments thereof or an anti-CD2 antibody immobilized on a surface, or by contacting a protein kinase C activator (for example, bryostatin) and calcium ionophore.

There are two splice variants for Claudin18 (CLDN18, CLD18), respectively splice variant 1 (CLDN18A1), gene registration number: NP_057453, NM016369; and splice variant 2 (CLD18A2), gene registration number: NM_001002026, NP_001002026. Claudin18 is an intrinsic transmembrane protein located in the tight junction between epithelium and endothelium. In some embodiments, the claudin 18A2 or claudin 18A2 peptide or Claudin 18.2 is a peptide comprising the amino acid sequence of SEQ ID NO: 54 or a protein/peptide of a variant of the amino acid sequence. The term “variant” refers to a mutant, splice variant, conformation, isoform, allelic variant, species variant and species homolog, especially naturally occurring variant. The allelic variant involves changes in the normal sequence of a gene, the significance of which is usually not obvious. Whole-gene sequencing usually identifies a large number of allelic variants of a given gene. An interspecies homolog is a nucleic acid or amino acid sequence that has a different species origin from a given nucleic acid or amino acid sequence.

CLD18A2 is strongly expressed in several cancer types, including gastric cancer, esophageal cancer, pancreatic cancer and lung cancer such as non-small cell lung cancer, ovarian cancer, colon cancer, liver cancer, head and neck cancer and gallbladder cancer, gastric cancer metastasis such as Krukenberg tumor, peritoneal metastasis and lymph node metastatic to lung tumors and human cancer cell lines, and mainly expressed in the adenocarcinoma subtypes of these indications, therefore CLDN18A2 is particularly suitable for antibody-mediated prevention and/or treatment of primary tumors and metastasis targets thereof. In one example, the antigen binding portion of TFP recognizes and binds to gastric cancer, esophageal cancer, pancreatic cancer, and lung cancer, such as non-small cell lung cancer, ovarian cancer, colon cancer, liver cancer, head and neck cancer, and gallbladder cancer, and gastric cancer metastasis such as Krukenberg tumor, peritoneal metastasis and lymph node metastasis to lung tumors and epitopes in the extracellular domain of CLD18A2 expressed on human cancer cell lines.

The term “GPC3” or “Glypican 3” is a member of the Glypican family, which plays an important role in regulating cell growth and differentiation. An abnormal expression of GPC3 is closely related to the occurrence and development of a variety of tumors, such as liver cancer, lung cancer, breast cancer, ovarian cancer, kidney cancer, thyroid cancer, gastric cancer, colorectal cancer, and so on.

The term “immune effector cell” refers to a cell that exerts an effector function during an immune response, including, for example, immune cells secreting cytokines and/or chemokines, killing microorganisms, secreting antibodies, and recognizing or eliminating tumor cells. In some embodiments, immune effector cells include T cells (cytotoxic T cells, helper T cells, tumor-infiltrating T cells), B cells, natural killer cells, neutrophils, macrophages, and dendritic cells.

The term “immune effector function” includes any function mediated by the composition of the immune system, which can lead to inhibition of tumor growth and/or inhibition of tumorigenesis, including inhibition the spread and metastasis of a tumor. Preferably, the immune effector function kills tumor cells. Preferably, the immune effector function in the present invention is antibody-mediated, including complement-dependent cytotoxicity (CDC), antibody-dependent cell-mediated cytotoxicity (ADCC), antibody-dependent cell-mediated phagocytosis (ADCP), inducing apoptosis in cells carrying tumor-associated antigens (for example, through the binding of antibodies to surface antigens), inhibiting CD40L-mediated signaling (for example, binding to CD40 receptors or CD40 ligands (CD40L) through the antibodies), and/or inhibiting the proliferation of cells carrying tumor-associated antigens, preferably ADCC and/or CDC. Therefore, antibodies capable of mediating one or more immune effector functions are preferably capable of inducing CDC-mediated lysis, ADCC-mediated lysis, apoptosis, homo-adhesion and/or phagocytosis (preferably by inducing CDC-mediated Lysis and/or ADCC-mediated lysis) to mediate the killing of tumor cells. Antibodies can also function simply by binding to tumor-associated antigens on the surface of cancer cells. For example, antibodies can block the function of tumor-associated antigens or induce apoptosis by binding to tumor-associated antigens on the surface of tumor cells.

The term “antigen presenting cell” or “APC” refers to a cell in the immune system that display a complex of foreign antigens and major histocompatibility complex (MHC) on the surface, such as helper cells (such as B cells, dendritic cells, etc.). T cells can recognize these complexes using T cell receptor (TCR) thereof. APC processes the antigen and presents it to T cells.

The term “anti-tumor effect” refers to a biological effect that can be manifested in various ways, including but not limited to, for example, reduction in tumor volume, reduction in the number of tumor cells, reduction in the number of metastases, increase in life expectancy, reduction in tumor cell proliferation, and reduction in tumor cell survival rate, or improvement in various physiological symptoms related to cancerous conditions. The “anti-tumor effect” can also be expressed by the ability of the peptides, polynucleotides, cells and antibodies of the present invention to prevent tumorigenesis.

The term “autologous” refers to any material derived from an individual that will later be reintroduced into that same individual.

The term “allogeneic” refers to any material derived from a different animal or a different patient of the same species as the individual into which the material is introduced. When the genes at one or more loci are different, two or more individuals are considered to be allogeneic to each other. In some aspects, allogeneic materials from individuals of the same species may be genetically different enough for antigenic interaction to occur.

The term “xenogeneic” refers to animals in which the grafts are derived from different species.

The term “genetically engineered cell” refers to a cell modified by means of genetic engineering.

The terms “therapeutically effective amount”, “therapeutically effective”, “effective amount” or “in an effective amount” are used interchangeably herein and refer to the amount of a compound, preparation, substance or composition that is effective to achieve a specific biological result as described herein, such as but not limited to an amount or dose sufficient to promote T cell response. When indicating “immunologically effective amount”, “anti-tumor effective amount”, “tumor-suppressing effective amount” or “therapeutically effective amount”, the precise number of immune effector cells and therapeutic agents of the present invention to be administered can be determined by a physician in consideration of the individual's age, weight, tumor size, degree of infection or metastasis, and the condition of a patient (subject). An effective amount of immune effector cells refers to, but is not limited to, the number of immune effector cells which can increase, enhance or prolong the anti-tumor activity of immune effector cells; increase the number of anti-tumor immune effector cells or activated immune effector cells; promote IFN-γ secretion, tumor regression and tumor shrinkage and tumor necrosis.

CD3 (Cluster of Differentiation 3) T cell co-receptor is a protein complex composed of four different chains. In mammals, the complex contains one CD3γ chain, CD3δ chain, and two CD3ε chains. These chains have a molecule of accessory T cell receptor (TCR) and zeta-chain to generate activation signals for T lymphocytes. The TCR, ζ chain and CD3 molecule together constitute a T cell receptor complex. The CD3 molecule is connected to the T cell receptor (TCR) through a salt bridge to form a TCR-CD3 complex, which participates in the signaling of T cells, and is mainly used to label thymocytes, T lymphocytes and T cell lymphomas. The cytoplasmic segment of CD3 contains immunoreceptor tyrosine-based activation motif (ITAM). TCR recognizes and binds to the antigen peptide presented by the MHC (major histo-compatibility complex) molecule, resulting in the tyrosine residues in the conserved sequence of ITAM of CD3. being phosphorylated by the tyrosine protein kinase p56lck in T cells, and then recruiting other tyrosine protein kinases (such as ZAP-70) containing SH2 (Scr homology 2) domain. The phosphorylation of ITAM and the binding to ZAP-70 are one of the important biochemical reactions in the early stages of the signaling process of T cell activation. Therefore, the function of the CD3 molecule is to transduce the activation signal generated by the TCR to recognize the antigen. In this application, the exogenous receptor that can bind to the target antigen and trigger CD3 signal activation includes at least one CD3 binding site and at least one additional antigen binding site specific to bacterial substance, viral protein, autoimmune marker, or antigen present specific cells (e.g., cell surface proteins of B cells, T cells, natural killer (NK) cells, bone marrow cells, phagocytes, or tumor cells). Such exogenous receptors can cross-link two kinds of cells and can be used to direct T cells to specific targets and trigger the cytotoxic activity of T cells on the target cells. Examples of such targets may be tumor cells or infectious agents, such as viral pathogens or bacterial pathogens.

The term “stimulation” refers to a primary response induced by the binding of a stimulation domain or a stimulating molecule (eg, TCR/CD3 complex) to its cognate ligand, thereby mediating signaling events, such as, but not limited to, signaling via TCR/CD3 complex. The stimulation can mediate changes in the expression of certain molecules and/or reorganization of the cytoskeleton structure.

The term “stimulatory molecule” or “stimulatory domain” refers to a molecule or a part thereof expressed by T cells, which provides a primary cytoplasmic signaling sequence that modulates the primary activation of the TCR complex in a stimulating manner for at least some aspects of the T cell signaling pathway. In one aspect, the primary signal is initiated by, for example, the binding of the TCR/CD3 complex to the peptide-loaded MHC molecule, and it leads to the mediation of T cell responses including but not limited to proliferation, activation, differentiation, etc. Primary cytoplasmic signaling sequences that act in a stimulating manner (also referred to as “primary signaling domains”) may contain signaling motifs, which are called immunoreceptor tyrosine-based activation motifs or “ITAM”. Examples of ITAM-containing primary cytoplasmic signaling sequences that are particularly useful in the present invention include but are not limited to those derived from TCRζ, FcRγ, FcRβ, CD3γ, CD3δ, CD3ε, CD5, CD22, CD79a, CD79b, CD278 (also known as “ICOS”) and CD66d.

The term “intracellular signaling domain” refers to the intracellular part of a molecule. Intracellular signaling domains generate signals that promote immune effector functions of TFP-containing cells, such as T cells expressing TFP. For example, examples of immune effector functions in T cells expressing TFP include cytolytic activity and T helper cell activity, including secretion of cytokines. In one embodiment, the intracellular signaling domain may comprise a primary intracellular signaling domain. Exemplary primary intracellular signaling domains include intracellular signaling domains derived from molecules responsible for primary stimulation or antigen-dependent stimulation. In one embodiment, the intracellular signaling domain may comprise a costimulatory intracellular domain. Exemplary costimulatory intracellular signaling domains include intracellular signaling domains derived from molecules responsible for costimulatory signals or antigen-independent stimulation.

The primary intracellular signaling domain may contain ITAM (“immunoreceptor tyrosine-based activation motif”). Examples of ITAM-containing primary cytoplasmic signaling sequences include, but are not limited to, those derived from CD3ζ, FcRγ, FcRβ, CD3γ, CD3δ, CD3ε, CD5, CD22, CD79a, CD79b, and CD66d DAP10 and DAP12.

The term “costimulatory molecule” refers to a homologous binding partner on T cells, which specifically binds to a costimulatory ligand, thereby mediating the costimulatory response of T cells, such as but not limited to proliferation. Co-stimulatory molecules are cell surface molecules other than antigen receptors or their ligands required for an effective immune response. Costimulatory molecules include but are not limited to MHC class 1 molecules, BTLA and Toll ligand receptors, as well as DAP10, DAP12, CD30, LIGHT, OX40, CD2, CD27, CD28, CDS, ICAM-1, LFA-1 (CD11a/CD18) and 4-1BB (CD137). The costimulatory intracellular signaling domain can be the intracellular part of a costimulatory molecule. Costimulatory molecules can be represented in the following protein families: TNF receptor proteins, immunoglobulin-like proteins, cytokine receptors, integrins, signaling lymphocyte activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function associated antigen 1 (LFA-1), CD2, CD7, LIGHT, NKG2C, SLAMF7, NKp80, CD160, B7-H3 and ligands that specifically bind to CD83. The intracellular signaling domain may comprise the entire intracellular part of the molecule from which it is derived or the entire natural intracellular signaling domain, or a functional fragment thereof. The term “4-1BB” refers to a member of the TNFR superfamily, which has the amino acid sequence provided under GenBank accession number AAA62478.2. or equivalent residues from non-human species such as mice, rodents, monkeys, apes, etc.; and “4-1BB costimulatory domain” is defined as amino acid residues 214-255 of GenBank accession number AAA62478.2. or equivalent residues from non-human species such as mice, rodents, monkeys, apes, etc.

The term “encoding” refers to the inherent properties of polynucleotides such as genes, cDNAs, or mRNAs in which specific nucleotide sequences are used as templates for the synthesis of other polymers and macromolecules in biological processes. The polymers and macromolecules have a certain Nucleotide sequence (e.g., rRNA, tRNA, and mRNA) or defined amino acid sequence and the resulting biological properties. Therefore, if the transcription and translation of mRNA corresponding to a gene produces a protein in a cell or other biological system, the gene, cDNA, or RNA encodes the protein. The coding strand and its nucleotide sequence are identical to the mRNA sequence and are usually provided in the sequence listing, while the non-coding strand used as a template for the transcription of a gene or cDNA can be referred to as a coding protein or other products of the gene or cDNA. Unless otherwise specified, “nucleotide sequence encoding an amino acid sequence” includes all nucleotide sequences that are degenerate forms of each other and encode the same amino acid sequence. The phrase “nucleotide sequence” encoding a protein or RNA may also include introns to the extent that the nucleotide sequence encoding the protein may contain one or more introns in some forms.

The term “expression” refers to the transcription and/or translation of a specific nucleotide sequence driven by a promoter.

The term “transfer vector” refers to a composition containing an isolated nucleic acid and a substance that can be used to deliver the isolated nucleic acid to the inside of a cell. Many vectors are known in the art, including but not limited to linear polynucleotides, polynucleotides associated with ionic or amphiphilic compounds, plasmids, and viruses. Therefore, the term “transfer vector” includes autonomously replicating plasmids or viruses. The term should also be interpreted to further include non-plasmid and non-viral compounds that facilitate the transfer of nucleic acids into cells, such as polylysine compounds, liposomes, and the like. Examples of virus transfer vectors include, but are not limited to, adenoviral vectors, adeno-associated virus vectors, retroviral vectors, lentiviral vectors, and the like.

The term “expression vector” refers to a vector comprising a recombinant polynucleotide comprising an expression control sequence operably linked to the nucleotide sequence to be expressed. The expression vector contains sufficient cis-acting elements for expression; other elements for expression can be provided by the host cell or in an in vitro expression system. Expression vectors include all expression vectors known in the art, including cosmids, plasmids (for example, naked or contained in liposomes), and viruses incorporating recombinant polynucleotides (for example, lentivirus, retrovirus, adenovirus). Virus and adeno-associated virus).

The term “homology” or “identity” refers to the identity of subunit sequence between two polymer molecules, for example, between two nucleic acid molecules, such as two DNA molecules or two RNA molecules, or between two polypeptide molecules. When subunit positions in two molecules are occupied by the same monomer subunit; for example, if the position of each of two DNA molecules is occupied by adenine, they are homologous or identical at that position. The homology between two sequences is a direct function of the number of matching or homologous positions; for example, if half of the positions in the two sequences (for example, 5 positions in a polymer of 10 subunits in length) are homologous, the two sequences are 50% homologous; if 90% of the positions (for example, 9 out of 10) are matched or homologous, then the two sequences are 90% homologous.

In the context of two or more nucleic acid or polypeptide sequences, identity percent refers to two or more sequences that are the same. When comparing and aligning for maximum correspondence in a comparison window or a designated area, as measured by using one of the following sequence comparison algorithms or by manual alignment and visual inspection, if the two sequences have a specified percentage of identical amino acid residues or nucleotides (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or if not specified, over the entire sequence), then the two sequences are “substantially the same”. Optionally, the identity exists over a region of at least about 50 nucleotides (or 10 amino acids) in length, or more preferably over a region of 100 to 500 or 1000 or more nucleotides in length (Or 20, 50, 200 or more amino acids). For sequence comparison, usually a sequence serves as a reference sequence against which the test sequence is compared. When a sequence comparison algorithm is used, a test sequence and a reference sequence are input into a computer, and the sub-sequence coordinates and the sequence algorithm program parameters are specified, if necessary. Default program parameters can be used, or alternative parameters can be specified. Subsequently, the sequence comparison algorithm calculates the percent sequence identity of the test sequence relative to the reference sequence based on the program parameters. Methods of sequence alignment for comparison are well known in the art. In one aspect, the invention contemplates modification of the amino acid sequence of the starting antibody or fragment (e.g., scFv) that produces a functionally equivalent molecule. For example, the anti-GPC3 or Claudin 18.2 binding domain contained in TFP, such as the VH or VL of scFv, can be modified to retain at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to the initial VH or VL framework region of the anti-GPC3 or Claudin 18.2 binding domain, such as scFv. The present invention considers the modification of the entire TFP construct, for example, the modification on one or more amino acid sequences of each domain of the TFP construct, for producing a functionally equivalent molecule. The TFP construct can be modified to retain at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity of the starting TFP construct.

In a specific embodiment, the amino acid sequences of the anti-GPC3 antibody that can be used in the present invention and its CDR sequence, heavy chain variable region and light chain variable region are shown in SEQ ID NO: 23, 26-33 and 137-208. In a preferred embodiment, the amino acid sequences of the anti-GPC3 antibody used in the present invention and it's CDR sequence, heavy chain variable region and light chain variable region are shown in SEQ ID NOs: 23 and 26-33.

In one embodiment, the amino acid sequences of the anti-Claudin 18.2 antibody that can be used in the present invention and its CDR sequence, heavy chain variable region and light chain variable region are shown in SEQ ID NO: 1, 15-22 and 56-136. In a preferred embodiment, the amino acid sequences of the anti-Claudin 18.2 antibody used in the present invention and its CDR sequence, heavy chain variable region and light chain variable region are shown in SEQ ID NO: 1 and 15-22.

The term “isolated” means changed or removed from the natural state. For example, a nucleic acid or peptide naturally present in a living animal is not “isolated”, but the same nucleic acid or peptide that is partially or completely separated from a substance co-existing in its natural state is “isolated.” The isolated nucleic acid or protein may exist in a substantially purified form or may exist in a non-natural environment such as a host cell.

The term “operably linked” or “transcription control” refers to a functional linkage between a regulatory sequence and a heterologous nucleic acid sequence, which results in the expression of the latter. For example, when the first nucleic acid sequence and the second nucleic acid sequence are arranged in a functional relationship, the first nucleic acid sequence and the second nucleic acid sequence are operably linked. For example, if a promoter affects the transcription or expression of a coding sequence, the promoter is operably linked to the coding sequence. The operably linked DNA sequences may be adjacent to each other, and for example, in the case where two protein coding regions need to be linked, the DNA sequences are in the same reading frame.

The term “nucleic acid” or “polynucleotide” refers to deoxyribonucleic acid (DNA) or ribonucleic acid (RNA) and polymers thereof in single-stranded or double-stranded form. Unless specifically defined, the term includes nucleic acids containing known analogs of natural nucleotides that have binding properties similar to the reference nucleic acid and are metabolized in a manner similar to naturally occurring nucleotides. Unless otherwise specified, a specific nucleic acid sequence also implicitly includes its conservatively modified variants (e.g., degenerate codon substitutions), alleles, orthologs, SNPs and complementary sequences, as well as explicitly indicated sequences. Specifically, degenerate codon replacement can be achieved by generating a sequence in which the third position of one or more selected (or all) codons is replaced by mixed bases and/or deoxyinosine residues.

The terms “peptide”, “polypeptide” and “protein” are used interchangeably and refer to a compound composed of amino acid residues covalently linked by peptide bonds. A protein or peptide must contain at least two amino acids, and there is no limit to the maximum number of amino acids that can constitute a protein or peptide sequence. Polypeptides include any peptide or protein comprising two or more amino acids connected to each other by peptide bonds. As used herein, the term refers to both short and long chains. Short chains are also commonly referred to in the art as peptides, oligopeptides, and oligomers, and long chains are commonly referred to as proteins in the art, and there are many types. “Polypeptide” includes, for example, biologically active fragments, substantially homologous polypeptides, oligopeptides, homodimers, heterodimers, polypeptide variants, modified polypeptides, derivatives, analogs, fusion proteins, and the like. Polypeptides include natural peptides, recombinant peptides or a combination thereof.

The term “promoter/regulatory sequence” refers to a nucleic acid sequence required to express a gene product operably linked to a promoter/regulatory sequence. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell The nucleotide sequence of the product.

“In vitro transcribed RNA” refers to RNA that has been synthesized in vitro, preferably mRNA. Generally, in vitro transcribed RNA is produced by an in vitro transcription vector. The in vitro transcription vector contains a template for producing in vitro transcribed RNA.

The term “antibody” refers to a protein or polypeptide sequence derived from an immunoglobulin molecule that specifically binds to an antigen. Antibodies can be polyclonal or monoclonal, multi-chain or single-chain, or whole immunoglobulins, and can be derived from natural sources or recombinant sources. The antibody may be a tetramer of immunoglobulin molecules.

The term “antibody fragment” refers to at least a portion of an antibody that retains the ability to specifically interact with an epitope of an antigen (e.g., through binding, steric hindrance, stabilization/destabilization, spatial distribution). Examples of antibody fragments include, but are not limited to, Fab, Fab′, F(ab′)₂, Fv fragments, scFv antibody fragments, disulfide-linked Fvs (sdFv), Fd fragments composed of VH and CH1 domains, linear antibodies, single domain antibodies such as sdAb (VL or VH), camelid VHH domains, multispecific antibodies formed by antibody fragments (e.g., bivalent fragments including two Fab fragments connected by disulfide bonds in the hinge region) and isolated CDR or other epitope binding fragments of antibodies. Antigen-binding fragments can also be incorporated into single domain antibodies, maximal antibodies, minibodies, nanobodies, intracellular antibodies, diabodies, tribodies, tetrabodies, v-NAR and double-scFv (see, for example, Hollinger and Hudson, “Nature Biotechnology” (23): 1126-1136, 2005).

The term “scFv” refers to a fusion protein comprising at least one antibody fragment including a light chain variable region and at least one antibody fragment including a heavy chain variable region, wherein the light chain and heavy chain variable regions are contiguous (For example, via a synthetic linker such as a short flexible polypeptide linker), and can be expressed as a single-chain polypeptide, and wherein the scFv retains the specificity of the intact antibody from which it is derived. Unless specified, as used herein, the scFv may have the VL and VH variable regions in any order (for example, relative to the N-terminus and C-terminus of the polypeptide), and the scFv may include VL-linker-VH or may include VH-Linker-VL.

The term “antibody heavy chain” refers to the larger of the two polypeptide chains present in the antibody molecule in its naturally occurring configuration and usually determines the type of antibody to which it belongs.

The term “antibody light chain” refers to the smaller of the two polypeptide chains present in an antibody molecule in its naturally occurring configuration. κ(k) and λ(l) light chains refer to two main isotypes of antibody light chains.

The term “recombinant antibody” refers to an antibody produced using recombinant DNA technology, such as, an antibody expressed by a phage or yeast expression system. The term should also be interpreted as referring to antibodies that have been produced by synthesizing a DNA molecule encoding the antibody (and wherein the DNA molecule expresses the antibody protein) or the amino acid sequence of the specified antibody, wherein the DNA or amino acid sequence has been obtained by using recombinant DNA or amino acid sequence technology is available and the well-known in the art.

A skilled person in the art will understand that the antibodies or antibody fragments of the present invention can be further modified so that there are changes in amino acid sequence (for example, relative to the wild type), but no change in desired activities. For example, additional nucleotide substitutions can be made to the protein, resulting in amino acid substitutions at “non-essential” amino acid residues. For example, a non-essential amino acid residue in the molecule can be replaced by another amino acid residue from the same side chain family. In another embodiment, the amino acid string may be replaced by a string that is similar in structure but different in sequence and/or composition from a member of the side chain family. For example, conservative substitutions may be made in which the amino acid residue is replaced by an amino acid residue having a similar side chain.

In the art, families of amino acid residues with similar side chains have been defined, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, Isoleucine) and aromatic side chains (e.g., tyrosine, phenylalanine, tryptophan, histidine).

The term “antigen” or “Ag” refers to a molecule that causes an immune response. The immune response may involve the production of antibodies or the activation of cells with specific immunity, or both. A skilled person in the art should understand that any macromolecule including virtually all proteins or peptides can serve as an antigen. In addition, the antigen can be derived from recombinant or genomic DNA. When the term is used herein, a skilled person in the art should understand that the term includes any DNA including a nucleotide sequence or part of a nucleotide sequence encoding a protein that causes an immune response, and therefore encoding an “antigen”. In addition, a skilled person in the art should understand that the antigen need not be encoded only by the full-length nucleotide sequence of the gene. It is obvious that the present invention includes, but is not limited to, the use of partial nucleotide sequences of more than one gene, and these nucleotide sequences are arranged in different combinations to encode polypeptides eliciting a desired immune response. Moreover, those skilled in the art should understand that the antigen does not need to be encoded by a “gene” at all. It is obvious that the antigen can be produced synthetically, or it can be derived from a biological sample, or it can be a macromolecule other than a polypeptide. Such biological samples may include, but are not limited to, tissue samples, tumor samples, cells or fluids with other biological components.

The term “antigen recognition unit” as used herein refers to immunoglobulin molecules and immunologically active parts of immune molecules, that is, a molecule that contains an antigen binding site that specifically binds to an antigen (“immune response”). The term “antigen recognition unit” also includes immunoglobulin molecules derived from various species, including invertebrates and vertebrates. Structurally, the simplest naturally occurring antibody (e.g., IgG) contains four polypeptide chains, two heavy (H) chains and two light (L) chains interconnected by disulfide bonds Immunoglobulins represent a large family of molecules including several types of molecules, such as IgD, IgG, IgA, IgM, and IgE. The term “immunoglobulin molecule” includes, for example, hybrid antibodies or modified antibodies and fragments thereof. It has been shown that the antigen-binding function of antibodies can be performed by fragments of naturally-occurring antibodies. These fragments are collectively referred to as “antigen recognition units”. The term “antigen recognition unit” also includes any molecular structure containing a polypeptide chain that has a specific shape that matches the epitope and recognizes the epitope, in which one or more non-covalent binding interactions stabilize the complex between the molecular structure and the epitope. Examples of the antigen recognition unit include Fab fragments, monovalent fragments consisting of VL, VH, CL and CH1 domains, and bivalent fragments (F(ab)2 fragments); Fd fragments composed of VH and CH1 domains, Fv fragments composed of single-arm VL and VH domains of antibodies; dAb fragments composed of VH domains (Ward et al., Nature, 341:544-546, 1989); and an isolated complementarity determining region (CDR) or any fusion protein containing such an antigen recognition unit.

If the antigen recognition unit binds to an antigen with greater affinity or avidity compared with binding with other reference antigens (including polypeptides or other substances), the antigen recognition unit “specifically binds” to the antigen or is “immunoreactive with the antigen”.

“Tumor antigen” refers to an antigen common to specific hyperproliferative diseases. In certain aspects, the hyperproliferative disorder antigens of the invention are derived from cancer. The tumor antigens of the present invention include but are not limited to: Thyroid Stimulating Hormone Receptor (TSHR); CD171; CS-1; C-type lectin-like molecule-1; Ganglioside GD3; Tn antigen; CD19; CD20; CD 22; CD 30; CD 70; CD 123; CD 138; CD33; CD44; CD44v7/8; CD38; CD44v6; B7H3 (CD276), B7H6; KIT (CD117); Interleukin 13 receptor subunit α (IL-13Rα); Interleukin 11 receptor alpha (IL-11Rα); prostate stem cell antigen (PSCA); prostate specific membrane antigen (PSMA); carcinoembryonic antigen (CEA); NY-ESO-1; HIV-1 Gag; MART-1; gp100; Tyrosinase; Mesothelin; EpCAM; Protease Serine 21 (PRSS21); Vascular Endothelial Growth Factor Receptor, Vascular Endothelial Growth Factor Receptor 2 (VEGFR2); Lewis (Y) Antigen; CD24; Platelet Derived Growth Factor Receptor β (PDGFR-β); stage-specific embryonic antigen-4 (SSEA-4); cell surface-associated mucin 1 (MUC1), MUC6; epidermal growth factor receptor family and its mutants (EGFR, EGFR2, ERBB3, ERBB4, EGFRvIII); Neural cell adhesion molecule (NCAM); Carbonic anhydrase IX (CAIX); LMP2; Ephrin A receptor 2 (EphA2); Fucosyl GM1; Sialyl Lewis adhesion molecule (sLe); Ganglioside GM3(aNeu5Ac(2-3)bDGalp(1-4)bDGlcp(1-1)Cer; TGS5; High molecular weight melanoma-associated antigen (HMWMAA); o-acetyl GD2 ganglioside (OAcGD2); Folate receptor; Tumor vascular endothelial marker 1 (TEM1/CD248); Tumor vascular endothelial marker 7 related (TEM7R); Claudin 6, Claudin 18.2, Claudin 18.1; ASGPR1; CDH16; 5T4; 8H9; αvβ6 Integrin; B cell maturation antigen (BCMA); CA9; kappa light chain; CSPG4; EGP2, EGP40; FAP; FAR; FBP; embryonic AchR; HLA-A1, HLA-A2; MAGEA1, MAGE3; KDR; MCSP; NKG2D ligand; PSC1; ROR1; Sp17; SURVIVIN; TAG72; TEM1; Fibronectin; Tenascin; Carcinoembryonic variant of tumor necrosis zone; G protein coupled receptor C class 5 group-member D (GPRCSD); X chromosome open reading frame 61 (CXORF61); CD97; CD17 9a; Anaplastic lymphoma kinase (ALK); polysialic acid; placenta specific 1 (PLAC1); hexose part of globoH glycoceramide (GloboH); breast differentiation antigen (NY-BR-1); uroplakin 2 (UPK2); Hepatitis A virus cell receptor 1 (HAVCR1); adrenergic receptor β3 (ADRB3); pannexin 3 (PANX3); G protein-coupled receptor 20 (GPR20); lymphocyte antigen 6 complex locus K9 (LY6K); Olfactory receptor 51E2 (OR51E2); TCRγ alternating reading frame protein (TARP); Wilms tumor protein (WT1); ETS translocation variant gene 6 (ETV6-AML); Sperm protein 17 (SPA17); X antigen family member 1A (XAGE1); Angiopoietin binds to cell surface receptor 2 (Tie2); Melanoma cancer testis antigen-1 (MAD-CT-1); Melanoma cancer testis antigen-2 (MAD-CT-2); Fos related antigen 1; p53 mutant; human telomerase reverse transcriptase (hTERT); sarcoma translocation breakpoint; melanoma inhibitor of apoptosis (ML-IAP); ERG (transmembrane protease serine 2 (TMPRSS2))ETS fusion gene); N-acetylglucosaminyltransferase V (NA17); Pairing box protein Pax-3 (PAX3); Androgen receptor; Cyclin B1; V-myc avian myeloidoma virus cancer Gene neuroblastoma-derived homolog (MYCN); Ras homolog family member C (RhoC); Cytochrome P450 1B1 (CYP1B1); CCCTC binding factor (zinc finger protein)-like (BORIS); recognized by T cells Squamous cell carcinoma antigen 3 (SART3); paired box protein Pax-5 (PAXS); proacrosin binding protein sp32 (OYTES1); lymphocyte-specific protein tyrosine kinase (LCK); A kinase anchor protein 4 (AKAP-4); Synovial sarcoma X breakpoint 2 (SSX2); CD79a; CD79b; CD72; Leukocyte-associated immunoglobulin-like receptor 1 (LAIR1); Fc fragment of IgA receptor (FCAR); Leukocyte immunoglobulin-like receptor Body subfamily member 2 (LILRA2); CD300 molecular-like family member f (CD300LF); C-type lectin domain family 12 member A (CLEC12A); bone marrow stromal cell antigen 2 (BST2); mucin-like hormone Receptor-like 2 containing EGF-like module (EMR2); Lymphocyte antigen 75 (LY75); Glypican-3 (GPC3); Fc receptor-like 5 (FCRL5); Immunoglobulin lambda-like polypeptide 1 (IGLL1).

The pathogen antigen is selected from: antigens from virus, bacteria, fungus, protozoa, or parasite; and the virus antigen is selected from: cytomegalovirus antigen, Epstein-Barr virus antigen, human immunodeficiency virus antigen, or influenza virus antigen.

The term “tumor heterogeneity” means that, after multiple divisions and proliferation during the growth of a tumor, daughter cells of the tumor its show molecular biological or genetic changes, so that there are differences in the growth rate, invasion ability, and drug sensitivity, prognosis and other aspects of the tumor. It is one of the characteristics of malignant tumors.

The term “cancer” refers to a broad category of disorders characterized by hyperproliferative cell growth in vitro (e.g., transformed cells) or in vivo. The conditions that can be treated or prevented by the method of the present invention include, for example, various neoplasms, including benign or malignant tumors, various hyperplasias, and the like. The method of the present invention can achieve the inhibition and/or reversal of the undesirable hyperproliferative cell growth involved in such conditions. Specific examples of cancer include, but are not limited to: blood cancer, colon cancer, rectal cancer, renal cell carcinoma, liver cancer, non-small cell carcinoma of the lung, small intestine cancer, esophageal cancer, melanoma, bone cancer, pancreatic cancer, skin cancer, Head and neck cancer, skin or intraocular melanoma, uterine cancer, ovarian cancer, rectal cancer, anal cancer, stomach cancer, testicular cancer, uterine cancer, fallopian tube cancer, endometrial cancer, cervical cancer, vagina cancer, vaginal cancer, Hodgkin's disease, non-Hodgkin's lymphoma, endocrine system cancer, thyroid cancer, parathyroid cancer, adrenal cancer, soft tissue sarcoma, urethral cancer, penile cancer, childhood solid tumors, bladder cancer, kidney or ureter cancer, Renal pelvis cancer, central nervous system (CNS) tumor, primary CNS lymphoma, tumor angiogenesis, spinal tumor, brain stem glioma, pituitary adenoma, Kaposi's sarcoma, epidermoid carcinoma, squamous cell carcinoma, T-cell lymphoma, environmentally induced cancer, a combination of the cancers and the metastatic foci of the cancers.

The term “transfected” or “transformed” or “transduced” refers to the process by which exogenous nucleic acid is transferred or introduced into a host cell. A “transfected” or “transformed” or “transduced” cell is a cell that has been transfected, transformed or transduced with exogenous nucleic acid. The cells include primary cell of a subject and progenies thereof.

The term “specifically binds” refers to an antibody or ligand that recognizes and binds a protein of a binding partner (such as a tumor antigen) present in a sample, but the antibody or ligand does not substantially recognize or bind to other molecules in the sample.

“Refractory” as used herein refers to a disease, such as cancer, which does not respond to treatment. In an embodiment, a refractory cancer may be resistant to treatment before or at the beginning of the treatment. In other embodiments, a refractory cancer may become resistant during treatment. Refractory cancers are also called resistant cancers. In the present invention, refractory cancers include, but are not limited to, cancers that are not sensitive to radiotherapy, relapse after radiotherapy, are not sensitive to chemotherapy, relapse after chemotherapy, are not sensitive to CAR-T treatment, or relapse after treatment. Refractory or recurrent malignant tumors can use the treatment regimens described herein.

“Relapsed” as used herein refers to the return of the signs and symptoms of a disease (e.g. cancer) or the return of a disease such as cancer during a period of improvement, for example, after a therapy, such as a previous treatment of cancer therapy.

The terms “individual” and “subject” have the same meaning herein, and can be a human and animal from other species. A “patient” is a subject who has a disease, disorder, or condition, or is at risk of suffering from a disease, disorder, or condition, or is otherwise in need of the compositions and methods provided herein.

The term “enhancement” refers to allowing a subject or tumor cell to improve its ability to respond to the treatment disclosed herein. For example, an enhanced response may include an increase of 5%, 10%, 15%, 20%, 25%, 30%, 35%, 40%, 45%, 50%, 55%, 60%, 65%, 70%, 75%, 80%, 85%, 90%, 95%, or 98% or higher in responsiveness. As used herein, “enhancing” can also refer to increasing the number of subjects responding to treatment, such as immune effector cell therapy. For example, an enhanced response may refer to the total percentage of subjects responding to treatment, where the percentages are 5%, 10%, 15%, 20%, 25%, 30%, 35%, 40%, 45%, 50%, 55%, 60%, 65%, 70%, 75%, 80%, 85%, 90%, 95% or 98% more.

In one aspect, treatment is judged by clinical results, and can also be based on the increase, enhancement or extension of the anti-tumor activity of T cells, for example the increase in the number of anti-tumor T cells or activated T cells promotes IFN-γ secretion, both of them, as compared with the number before treatment. In another aspect, the clinical outcome is tumor regression; tumor shrinkage; tumor necrosis; anti-tumor response through the immune system; tumor enlargement, recurrence or spread, or a combination thereof. In an additional aspect, the therapeutic effect is predicted by the presence of T cells, the presence of genetic markers indicative of T cell inflammation, promotion of IFN-γ secretion, or a combination thereof.

The cells as disclosed herein can be administered to an individual by various routes, including, for example, oral or parenteral, such as intravenous, intramuscular, subcutaneous, intraorbital, intrasaccular, intraperitoneal, intrarectal, intracisternal, intratumoral, intravasal, intradermal administration, or passive or promoted absorption through the skin using, for example, skin patches or transdermal iontophoresis, respectively.

The total amount of agent to be administered in practicing the method of the present invention can be administered to the subject as a single dose as a bolus or by infusion over a relatively short period of time, or can be administered using a graded treatment regimen, wherein multiple doses are administered in segments. A skilled person will know that the amount of the composition to treat a pathological condition in a subject depends on many factors, including the age and general health of the subject, as well as the route of administration and the number of treatments to be administered. Based on these factors, a skilled person will adjust the specific dosage as needed. Generally, phase I and phase II clinical trials are initially used to determine the formulation of the composition as well as the route and frequency of administration.

Range: Throughout this disclosure, various aspects of the invention may be presented in a range. It should be understood that the description of a range is merely for convenience and brevity, and should not be regarded as an unchangeable limitation on the scope of the present invention. Therefore, the description of a range should be considered as specifically disclosing all possible subranges and individual values within that range. For example, the description of a range such as from 1 to 6 should be considered to specifically disclose subranges such as 1 to 3, 1 to 4, 1 to 5, 2 to 4, 2 to 6, 3 to 6, etc., and individual values within the range, such as 1, 2, 2.7, 3, 4, 5, 5.3, and 6. As another example, a range such as 95-99% identity includes a range with 95%, 96%, 97%, 98%, or 99% identity, and includes sub-ranges such as 96-99%, 96-98%, 96-97%, 97-99%, 97-98% and 98-99% identity. This applies regardless of the width of the range.

Based on the present disclosure, a skilled person should understand that many changes can be made in the disclosed specific embodiments and still obtain the same or similar results without departing from the spirit and scope of the present invention. The present invention is not limited in scope to the specific embodiments described herein (which are only intended to exemplify aspects of the present invention), and functionally equivalent methods and components shall fall within the scope of the present invention. In fact, the various modifications of the present invention as well as those shown and described herein will become apparent to a skilled person based on the foregoing description.

GPC3 and GPC3-Positive Tumors

As used herein, “GPC3” or “Glypican 3” is a member of the Glypican family, with gene registration numbers: NM_016697.3, NP_057906.2, which play an important role in regulating cell growth and differentiation. Abnormal expression of GPC3 is closely related to the occurrence and development of a variety of tumors, such as liver cancer, lung cancer, breast cancer, ovarian cancer, kidney cancer, thyroid cancer, gastric cancer, colorectal cancer, and so on.

In the present invention, immune effector cells target tumors that positively express GPC3. In a specific embodiment, the tumor includes, but is not limited to, liver cancer, stomach cancer, lung cancer, esophageal cancer, head and neck cancer, bladder cancer, ovarian cancer, cervical cancer, kidney cancer, pancreatic cancer, cervical cancer, liposarcoma, melanoma, adrenal carcinoma, schwannoma, malignant fibrous histiocytoma, esophageal cancer. A skilled person will know that some tumor cells, such as liver cancer cells, are not sensitive to many drugs. Therefore, even drugs that are effective in vitro may have poor effects in vivo, or even no effect. Therefore, in a preferred embodiment, the GPC3-positive tumors or GPC-positive tumors described herein are liver cancer, gastric cancer, lung cancer, and esophageal cancer.

TCR Modified T Cells

The present invention also provides TCR-modified T cells, which are transduced with a nucleic acid encoding the TCR or with the aforementioned recombinant plasmid containing the nucleic acid, or a virus containing the plasmid. Conventional nucleic acid transduction methods in the art, including non-viral and viral transduction methods, can be used in the present invention. Non-viral-based transduction methods include electroporation and transposon methods. Recently, the Nucleofector nuclear transfection instrument developed by Amaxa can directly introduce foreign genes into the nucleus to obtain efficient transduction of the target gene. In addition, the transduction efficiency of transposon systems based on Sleeping Beauty system or PiggyBac transposon is much higher than that of ordinary electroporation. The combined application of nucleofector transfection instrument and Sleeping Beauty transposon system has been reported [Davies J K., et al. Combining CD19 redirection and alloanergization to generate tumor-specific human T cells for allogeneic cell therapy of B-cell malignancies. Cancer Res, 2010, 70(10): OF1-10.], and this method not only has high transduction efficiency but also can realize the targeted integration of the target gene. In one embodiment of the present invention, the method for transduction of immune effector cells that achieve chimeric antigen receptor gene modification is a transduction method based on viruses, such as retroviruses or lentiviruses. The method has the advantages of high transduction efficiency, stable expression of foreign genes, and shortening the time for culturing immune effector cells to reach clinical level in vitro. On the surface of the transgenic immune effector cell, the transduced nucleic acid is expressed on its surface through transcription and translation. Through in vitro cytotoxicity experiments on various cultured tumor cells, it is proved that the immune effector cells modified by the chimeric antigen of the present invention have highly specific tumor cell killing effects (also known as cytotoxicity), and can be found in tumor tissues. Effectively survive. Therefore, the nucleic acid encoding the chimeric antigen receptor of the present invention, the plasmid containing the nucleic acid, the virus containing the plasmid, and the transgenic immune effector cells transduced with the nucleic acid, plasmid or virus of the present invention can be effectively used for tumor immunotherapy.

The TCR-modified T cells of the present invention can be applied to the preparation of pharmaceutical compositions or diagnostic reagents. In addition to including an effective amount of the immune cells, the composition may also include a pharmaceutically acceptable carrier. The term “pharmaceutically acceptable” means that when the molecular entities and compositions are properly administered to animals or humans, they will not produce adverse, allergic or other adverse reactions.

Specific examples of some substances that can be used as pharmaceutically acceptable carriers or components thereof are sugars, such as lactose, glucose, and sucrose; starches, such as corn starch and potato starch; cellulose and its derivatives, such as carboxymethyl fiber Sodium, ethyl cellulose and methyl cellulose; tragacanth powder; malt; gelatin; talc; solid lubricants, such as stearic acid and magnesium stearate; calcium sulfate; vegetable oils, such as peanut oil, cottonseed oil, Sesame oil, olive oil, corn oil, and cocoa butter; polyols, such as propylene glycol, glycerin, sorbitol, mannitol, and polyethylene glycol; alginic acid; emulsifiers, such as Tween®; wetting agents, such as sodium lauryl sulfate; coloring agent; flavoring agent; tablet pressing agent, stabilizer; antioxidant; preservative; pyrogen-free water; isotonic salt solution and phosphate buffer, etc.

The composition of the present invention can be made into various dosage forms according to needs, and the doctor can determine the beneficial dosage for the patient according to factors such as the patient's type, age, weight, general disease condition, and administration method. The mode of administration can be injection or other treatment methods.

### Advantages of the Present Invention

Compositions for using T cell receptor (TCR) fusion proteins to treat diseases, such as cancer and methods using the same are provided herein. As used herein, “T cell receptor (TCR) fusion protein” or “TFP” includes recombinant polypeptides derived from various polypeptides containing TCRs, which are generally capable of i) binding to a surface antigen on a target cell, and ii) interacting with other polypeptide components of the intact TCR complex, usually when co-located in or on the surface of the T cell. As provided herein, compared with a chimeric antigen receptor, TFP can not only inhibit the growth of tumor cells, but also release fewer cytokines, thereby effectively reducing the possibility of cytokine storms.

The present invention will be further explained below in conjunction with specific embodiments. It should be understood that these embodiments are only used to illustrate the present invention and not to limit the scope of the present invention. The experimental methods without specific conditions in the following examples usually follow the conventional conditions as described in J. Sambrook et al., Molecular Cloning Experiment Guide, Third Edition, Science Press, 2002, or according to the conditions described in the manufacturer The suggested conditions. All publications, patents, and patent applications mentioned in this specification are incorporated herein by reference to the extent that it is specifically and individually indicated that each individual publication, patent or patent application is incorporated by reference.

Exemplary antigen receptors of the present invention, including CAR, and methods for engineering and introducing receptors into cells, can refer to those disclosed in, for example, CN107058354A, CN107460201A, CN105194661A, CN105315375A, CN105713881A, CN106146666A, CN106519037A, CN106554414A, CN105331585A, CN106397593A, CN106467573A, CN104140974A, WO2017186121A1, WO2018006882A1, WO2015172339A8, and WO2018/018958A1.

### Example 1. Construction of T Cells Expressing TCR Fusion Protein

Using conventional molecular biology methods in the art, the scFv used in this example was an antibody targeting claudin 18.2, the amino acid sequence of which is shown in SEQ ID NO:1.

1. Construction of Plasmid

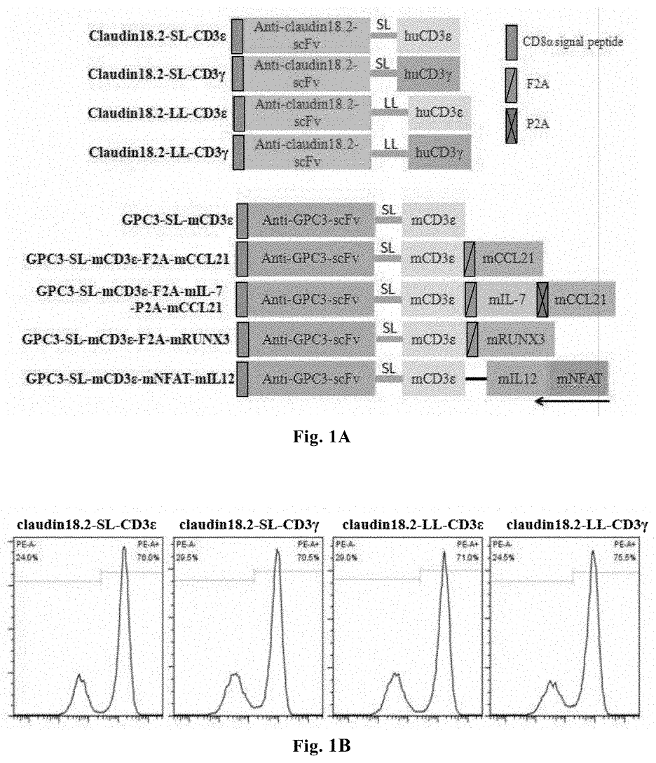

PRRLSIN-cPPT.EF-1α (purchased from addgene) was used as a vector, 4 different anti-Claudin18.2 lentiviral plasmids were prepared by inserting anti-Caludin18.2 single-chain antibody with CD3ε or CD3γ linked by different linkers in lengths (FIG. 1A).

pRRLSIN-cPPT.EF-1α-claudin18.2-LL-CD3ε, sequentially connected anti-cluadin18.2 single-chain antibody (SEQ ID NO: 1), long linker (SEQ ID NO: 2), CD3ε (SEQ ID NO: 3). Gene sequences of the three fragments, single-chain antibody claudin18.2 (SEQ ID NO: 4), long linker (SEQ ID NO: 5) and CD3ε (SEQ ID NO: 6) were joined together by bridge PCR, and double-digested by restriction enzymes MluI&SalI, thereby forming the fragment claudin18.2-LL-CD3ε (SEQ ID NO: 7). The vector was double-digested with restriction endonucleases MluI&SalI to obtain the linearized vector pRRL-MluI&SalI, and the homologous recombinase was used to circularize the vector and fragments to form the plasmid pRRL-claudin18.2-LL-CD3ε.

pRRLSIN-cPPT.EF-1α-claudin18.2-LL-CD3γ, sequentially connected anti-cluadin18.2 single-chain antibody cluadin18.2 (SEQ ID NO: 1), long linker (SEQ ID NO: 2), CD3γ (SEQ ID NO: 8). Gene sequences of the three fragments, single-chain antibody (SEQ ID NO: 4), long linker (SEQ ID NO: 5) and CD3γ (SEQ ID NO: 9) were joined together by bridging PCR, and double-digested by restriction enzymes MluI&SalI, thereby forming the fragment claudin18.2-LL-CD3γ (SEQ ID NO: 10). The vector was double-digested with restriction endonucleases MluI&SalI to obtain the linearized vector pRRL-MluI&SalI, and the homologous recombinase was used to circularize the vector and fragments to form the plasmid pRRL-claudin18.2-LL-CD3γ.

pRRLSIN-cPPT.EF-1α-claudin18.2-SL-CD3ε, sequentially connected anti-cluadin18.2 single-chain antibody cluadin18.2 (SEQ ID NO: 1), short linker (SEQ ID NO: 11), CD3ε (SEQ ID NO: 3). Gene sequences of the three fragments, single-chain antibody (SEQ ID NO: 4), short linker (SEQ ID NO: 12) and CD3ε (SEQ ID NO: 6) were joined together by bridging PCR, and double-digested by restriction enzymes MluI&SalI, thereby forming the fragment claudin18.2-SL-CD3ε (SEQ ID NO: 13). The vector was double-digested with restriction endonucleases MluI&SalI to obtain the linearized vector pRRL-MluI&SalI, and the homologous recombinase was used to circularize the vector and fragments to form the plasmid pRRL-claudin18.2-SL-CD3ε.

pRRLSIN-cPPT.EF-1α-claudin18.2-SL-CD3γ, sequentially connected anti-cluadin18.2 single-chain antibody cluadin18.2 (SEQ ID NO: 1), short linker (SEQ ID NO: 11), CD3γ (SEQ ID NO: 8). Gene sequences of the three fragments, single-chain antibody (SEQ ID NO: 4), short linker (SEQ ID NO: 12) and CD3γ (SEQ ID NO: 9) were joined together by bridging PCR, and double-digested by restriction enzymes MluI&SalI, thereby forming the fragment claudin18.2-SL-CD3γ (SEQ ID NO: 14). The vector was double-digested with restriction endonucleases MluI&SalI to obtain the linearized vector pRRL-MluI&SalI, and the homologous recombinase was used to circularize the vector and fragments to form the plasmid pRRL-claudin18.2-SL-CD3γ.

2. Preparation of T Cells Expressing TCR Fusion Protein

1) 293T cells were inoculated in a culture dish. Using conventional techniques in the field, plasmids pRRL-claudin18.2-LL-CD3ε, pRRL-claudin18.2-LL-CD3γ, pRRL-claudin18.2-SL-CD3ε, pRRL-claudin18.2-SL-CD3γ were transfected into 293T cells, respectively. After transfection for 72 hrs, the virus supernatant was collected to obtain lentivirus claudin18.2-LL-CD3ε, claudin18.2-LL-CD3γ, claudin18.2-SL-CD3ε, and claudin18.2-SL-CD3γ, respectively.

2) Peripheral blood mononuclear cells (PBMC) were separated from the blood of healthy donors using Ficoll (from GE) density gradient centrifugation method according to standard procedures. AIM-V medium (containing 2% human AB serum) was added at a density of about 1×10⁶/mL and CD3/CD28 activated magnetic beads (Invitrogen) at a cell: magnetic bead ratio of 1:1 and recombinant human IL-2 at a final concentration of 300 U/mL were added for stimulation for 48 hrs. Then T cells were infected with the recombinant lentivirus constructed above at MOI=10 to obtain T cells expressing TCR fusion protein: claudin18.2-SL-CD3ε cells, claudin18.2-SL-CD3γ cells, claudin18.2-LL-CD3ε cells, claudin18.2-LL-CD3γ cells.

The results of flow cytometry are shown in FIG. 1B, and all of the positive rates are over 70%.

The detection method for positive rate is: primary antibody: claudin18.2 antibody-Biotin-F(ab)2 (CARSGEN THERAPEUTICS CO., LTD.) at a concentration of 20 ug/ml, incubated on ice for 45 min, secondary antibody: Streptavidin PE (eBioscience) (1:200), incubated on ice for 45 min. Note: The primary antibody and secondary antibody were washed with PBS+1% FBS for 1 time.

### Example 2. In Vitro Killing Toxicity Test and In Vitro Cytokine Secretion Test

CytoTox 96 non-radioactive cytotoxicity detection kit (Promega) was used. The specific method refers to the instructions of CytoTox 96 non-radioactive cytotoxicity detection kit.

The number of target cells was (BxPC-3:15000/well; HGC-27:10000/well), and co-cultured with effector cells at an effector target ratio of 3:1, 1:1 or 1:3 for 18 hrs and detected (1640+5% FBS, 200 ul system), the effector cells were Untransduced (UTD) T cells, claudin18.2-28Z (the construction of which may refer to CN105315375A), claudin18.2-BBZ (the construction of which may refer to CN105315375A), claudin18.2-SL-CD3ε cells, claudin18.2-SL-CD3γ cells, claudin18.2-LL-CD3ε cells, claudin18.2-LL-CD3γ cells. The human claudin 18.2 fragment (SEQ ID NO: 54) was transferred into pancreatic cancer cell Bxpc-3 (purchased from ATCC) and gastric cancer cell HGC-27 (purchased from ATCC) to construct Bxpc-3-claudin 18.2 and HGC-27-claudin 18.2 cells expressing human claudin 18.2.

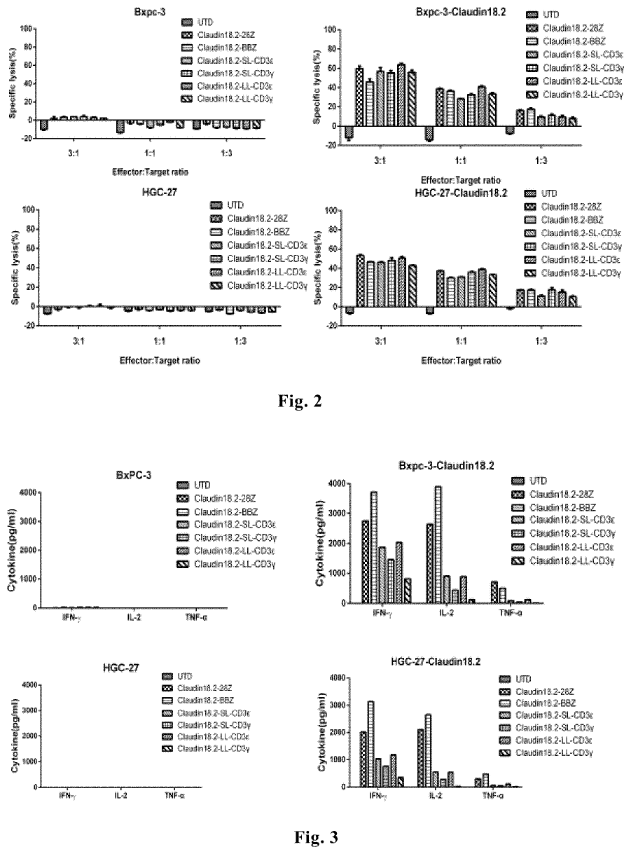

The experimental results are shown in FIG. 2. For Bxpc-3-claudin18.2 and HGC-27-claudin18.2 cells positive for the target antigen, all of claudin18.2-28Z, claudin18.2-BBZ and claudin18.2-SL-CD3ε cells, Claudin18.2-SL-CD3γ cells, claudin18.2-LL-CD3ε cells, claudin18.2-LL-CD3γ cells showed very significant specific cytotoxicities, and showed a gradient-dependent effect target ratio; that is, the higher the ratio, the stronger the cytotoxicity. While there is no specific cytotoxicity for Bxpc-3 and HGC-27 cells that do not express Claudin 18.2. Among them, when the effector target ratio is 3:1, the cytotoxicities of claudin18.2-28Z, claudin18.2-BBZ, claudin18.2-SL-CD3ε cells, claudin18.2-SL-CD3γ cells, claudin18.2-LL-CD3ε cells, claudin18 2-LL-CD3γ cells to Bxpc-3-claudin 18.2 were 59.66%, 45.77%, 59.51%, 57.34%, 63.91%, and 58.10%, respectively, and the cytotoxicities to HGC-27-claudin 18.2 were 46.18%, 47.93%, 50.56%, 42.71%, respectively. Compared with the second-generation of CART cells claudin18.2-28Z and claudin18.2-BBZ, the T cells expressing the TCR fusion protein have almost no difference in toxicities and killing effects on target cells.

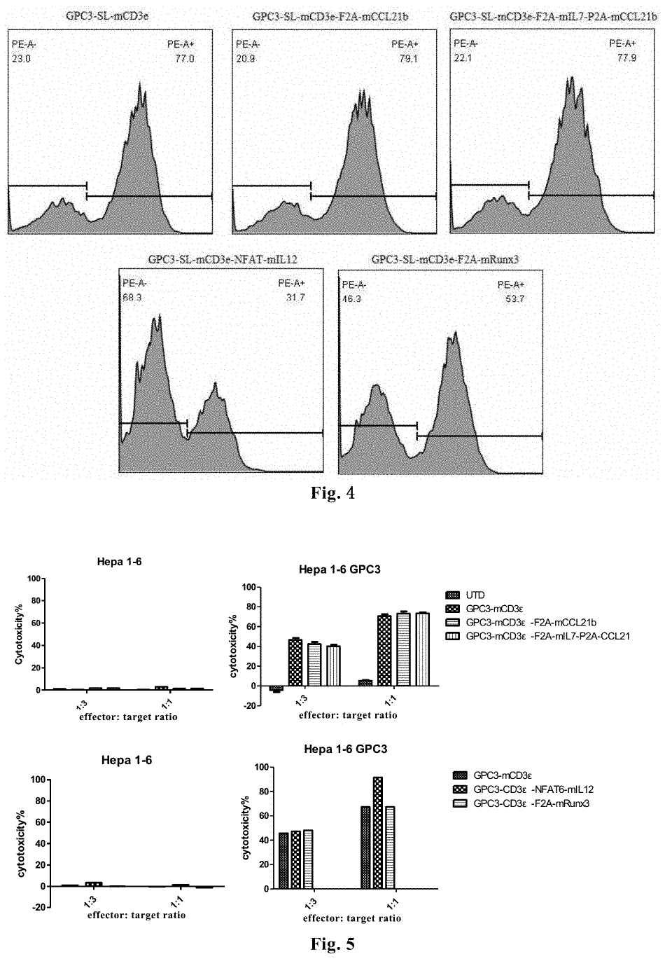

CBA kit (BD Biosciences) was used to detect the secretion of cytokines from T cells expressing TCR fusion protein after co-incubated with pancreatic cancer BxPC-3, BxPC-3-Claudin 18.2, and gastric cancer HGC-27, HGC-27-Claudin 18.2 for 24 hours at an effector target ratio of 1:1. The cytokine expression in the cell culture supernatant was detected. The results are shown in FIG. 3.

When co-incubated with target cells BxPC-3-Claudin18.2 and HGC-27-Claudin18.2 overexpressing Claudin 18.2, T cells expressing the TCR fusion protein can secrete large amounts of IFN-γ, IL-2 and TNF-α, in which, claudin18.2-SL-CD3γ cells and claudin18.2-LL-CD3γ secrete the three cytokines in lower amounts than CD3ε claudin18.2-SL-CD3ε cells and claudin18.2-LL-CD3ε cells (See Table 1). However, compared with the second-generation of CAR T cells claudin18.2-28Z and claudin18.2-BBZ, the amount of cytokine secreted by T cells expressing the TCR fusion protein was significantly reduced. When co-incubated with BxPC-3 and HGC-27 cells not expressing Claudin 18.2, the secretion of the above-mentioned cytokines was almost undetectable.

TABLE 1
Cytokine secretion of T cells expressing TCR fusion protein after co-incubation
with target cells

| | | | | | |
|---|---|---|---|---|---|
| | | | Claudin18.2- | Claudin18.2- | Claudin18.2- |
| Claudin18.2- | Claudin18.2- | Claudin18.2- | SL-CD3 | LL-CD3 | LL-CD3 |
| 28Z | BBZ | SL-CD3ε | γ | ε | γ |

BxPC-3-Claudin18.2

| | | | | | | |
|---|---|---|---|---|---|---|
| IFN-γ | 2751.61 | 3711.9 | 1873.35 | 1458.71 | 2030.58 | 812.55 |
| IL-2 | 2644.31 | 3895.33 | 900.06 | 440.31 | 885.75 | 117.06 |
| TNF- | | | | | | |
| α | 714.4 | 505.06 | 83.11 | 34.18 | 118.48 | 13.22 |

HGC27-Claudin18.

| | | | | | | |
|---|---|---|---|---|---|---|
| IFN-γ | 2007.27 | 3136.15 | 1033.04 | 761.53 | 1179.5 | 348.8 |
| IL-2 | 2113.24 | 2644.31 | 540.75 | 278.89 | 532.25 | 27.72 |
| TNF- | | | | | | |
| α | 300.05 | 466.75 | 62.91 | 45.11 | 107.4 | 13.85 |

### Example 3. Anti-Tumor Treatment Experiment of Subcutaneous Xenograft Tumor

NPG mouse of subcutaneous xenograft tumor with HGC27-Claudin18.2 gastric cancer cell

3×10⁶ of gastric cancer cells HGC27-Claudin18.2 were subcutaneously inoculated into the right axillary of female NPG mice (Beijing Weitongda Biotechnology Co., Ltd.), and the inoculation day was recorded as D0.

On D17 after subcutaneous inoculation of tumor tissue, the average tumor volume was about 270 mm³. The mice with xenograft tumor were divided into 5 groups: UTD group, claudin18.2-SL-CD3ε cell group, claudin18.2-SL-CD3γ cell group, claudin18.2-LL-CD3ε cell group and claudin18.2-LL-CD3γ cell group, and injected with the corresponding T cells expressing the TCR fusion protein respectively. The injection dosage was 5×10⁵ cells/animal. The UTD group was injected with 5×10⁵ cells/animal as untransduced T cell control.

After administration of T cells expressing TCR fusion protein, the volume of HGC27-Claudin18.2 xenograft tumor was measured every 3-4 days, and the changes in tumor volume of each group of mice were recorded. The tumor volume calculation formula is: (length×width²)/2. The results show that,

The mice were euthanized on D21 after the T cell expressing the TCR fusion protein were injected. Compared with the UTD group, the tumor inhibition rate of claudin18.2-SL-CD3ε cells was 40.42%, and the inhibition rates of claudin18.2-SL-CD3γ, claudin18.2-LL-CD3ε cells and claudin18.2-LL-CD3γ cells were lower than that of claudin18.2-SL-CD3ε cells. The changes in the body weight of the mice were recorded, and it was found that there was no significant difference in the body weight of the mice in each group. While in the liver cancer model of C57BL/6 mice with normal immune, compared with UTD group, the tumor inhibition rates of claudin18.2-SL-CD3γ, claudin18.2-LL-CD3ε, claudin18.2-LL-CD3γ, and claudin18.2-SL-CD3ε cells were about 25-45%.

### Example 4. Construction of T Cells Expressing TCR Fusion Protein and Targeting GPC3

Using conventional molecular biology methods in the art, the scFv used in this example is an antibody targeting GPC3, the amino acid sequence of which is shown in SEQ ID NO: 23, and the nucleotide sequence of which is shown in SEQ ID NO: 24.

1. Construction of Plasmid

pMSCV (purchased from addgene) was used as a vector, and an anti-GPC3 retroviral plasmid was formed by inserting an anti-GPC3 single-chain antibody GPC3 and CD3ε connected by a short linker.

The anti-GPC3 single-chain antibody (SEQ ID NO: 23), the short linker (SEQ ID NO: 11), and mCD3ε (SEQ ID NO: 34) were sequentially connected. Gene sequences of the three fragments, single-chain antibody (SEQ ID NO: 24), short linker (SEQ ID NO: 12) and mCD3ε (SEQ ID NO: 35) were linked together by bridge PCR to form the fragment GPC3-SL-mCD3ε (SEQ ID NO: 25) (FIG. 1A). The vector was double-digested with restriction enzymes EcoRI&HindIII to obtain a linearized vector pMSCV-EcoRI&HindIII, and the vector and fragments were circularized by the homologous recombinase to form a plasmid pMSCV-GPC3-SL-mCD3ε.

For GPC3-SL-mCD3ε, F2A (SEQ ID NO: 36) and mCCL21b (SEQ ID NO: 38) were sequentially connected. Gene sequences of F2A (SEQ ID NO: 37) and mCCL21b (SEQ ID NO: 39) were linked together by bridge PCR to form F2A-mCCL21b. The vector was double-digested with restriction enzymes EcoRI&HindIII to obtain a linearized vector pMSCV-EcoRI&HindIII, and the vector and fragments GPC3-SL-mCD3ε and F2A-mCCL21b were circularized by the homologous recombinase to form a plasmid pMSCV-GPC3-SL-mCD3ε-F2A-mCCL21b (FIG. 1A).

For GPC3-SL-mCD3ε, F2A (SEQ ID NO: 36) and mIL7 (SEQ ID NO: 40) were sequentially connected. Gene sequences of F2A (SEQ ID NO: 37) and mIL7 (SEQ ID NO: 41) were linked together by bridge PCR to form F2A-mIL7. Gene sequences of P2A (SEQ ID NO: 43) and mCCL21b (SEQ ID NO: 39) were linked together by bridge PCR to form P2A-mCCL21b. The vector was double-digested with restriction enzymes EcoRI&HindIII to obtain a linearized vector pMSCV-EcoRI&HindIII, and the vector and fragments GPC3-SL-mCD3ε, F2A-mIL7, P2A-mCCL21b were circularized by the homologous recombinase to form a plasmid pMSCV-GPC3-SL-mCD3ε-F2A-mIL7-P2A-mCCL21b (FIG. 1A).

For GPC3-SL-mCD3ε, NFAT (SEQ ID NO: 44), mIL2 Minimal Promoter (SEQ ID NO: 46), mIL12 (SEQ ID NO: 48) and PA2 (SEQ ID NO: 52) were connected, and gene sequences of the 4 fragments, NFAT (SEQ ID NO: 45), mIL2 Minimal Promoter (SEQ ID NO: 47), mIL12 (SEQ ID NO: 49) and PA2 (SEQ ID NO: 53) were linked together by bridge PCR to form NFAT-mIL12-PA2. The vector was double-digested with restriction enzymes EcoRI&HindIII to obtain a linearized vector pMSCV-EcoRI&HindIII. The vector and fragments GPC3-SL-mCD3ε and NFAT-mIL12-PA2 were circularized by a homologous recombinase to form a plasmid pMSCV-GPC3-SL-mCD3ε-NFAT-mIL12 (FIG. 1A).

For GPC3-SL-mCD3ε, F2A (SEQ ID NO: 36) and mRunx3 (SEQ ID NO: 50) were sequentially connected. Gene sequences of the two fragment, F2A (SEQ ID NO: 37) and mRunx3 (SEQ ID NO: 51) were joined by bridge PCR to form F2A-mRunx3. The vector was double-digested with restriction enzymes EcoRI&HindIII to obtain a linearized vector pMSCV-EcoRI&HindIII, and the digested vector and the fragments GPC3-SL-mCD3ε and F2A-mRunx3 were circularized by a homologous recombinase to form a plasmid pMSCV-GPC3-SL-mCD3ε-F2A-mRunx3 (FIG. 1A).

2. Construction of T Cells Expressing TCR Fusion Protein

1) 293T cells were inoculated in a culture dish. Using conventional techniques in the field, plasmids pMSCV-GPC3-SL-mCD38; pMSCV-GPC3-SL-mCD3ε-F2A-mCCL21b; pMSCV-GPC3-SL-mCD3ε-F2A-mIL7-P2A-mCCL21b; pMSCV-GPC3-SL-mCD3ε-NFAT-mIL12; or pMSCV-GPC3-SL-mCD3ε-F2A-mRunx3 were transfected into 293T cells. After 48 hours of transfection, the virus supernatant was collected to obtain the retrovirus GPC3-SL-mCD3ε, GPC3-SL-mCD3ε-F2A-mCCL21b, GPC3-SL-mCD3ε-F2A-mIL7-P2A-mCCL21b, GPC3-SL-mCD3ε-NFAT-mIL12, GPC3-SL-mCD3ε-F2A-mRunx3.

2) Mouse T cells were infected with the above retroviruses to obtain GPC3-SL-mCD3ε cells, GPC3-SL-mCD3ε-F2A-mCCL21b cells, GPC3-SL-mCD3ε-F2A-mIL7-P2A-mCCL21b cells, GPC3-SL-mCD3ε-NFAT-mIL12 cells, GPC3-SL-mCD3ε-F2A-mRunx3 cells.

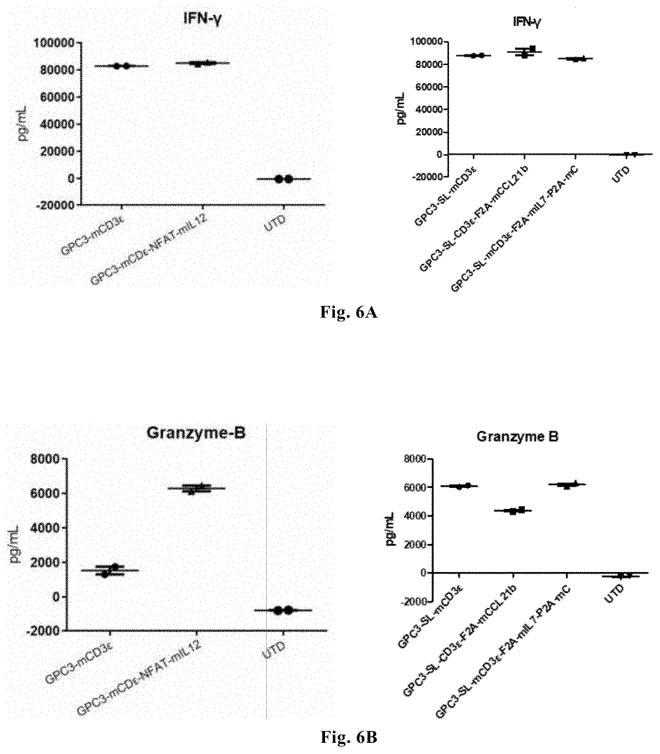

Results of the positive rate test are shown in FIG. 4. The positive rates of GPC3-SL-mCD3ε, GPC3-SL-mCD3ε-F2A-mCCL21b, GPC3-SL-mCD3ε-F2A-mIL7-P2A-mCCL21b is over 70%. The positive rate of GPC3-SL-mCD3ε-NFAT-mIL12 is over 30%. The positive rate of GPC3-SL-mCD3ε-F2A-mRunx3 cells is over 50%.

The detection method for positive rate is: Primary antibody: Anti-GPC3 antibody-Biotin-F(ab)2 (CARSGEN THERAPEUTICS CO., LTD.) at a concentration of 20 ug/ml, incubated on ice for 45 mins, secondary antibody: Streptavidin PE (1:200), incubated on ice for 45 mins.

### Example 5. In Vitro Killing Toxicity Test and In Vitro Cytokine Secretion Test

CytoTox 96 non-radioactive cytotoxicity detection kit (Promega) was used. The specific method can be found in the instructions of CytoTox 96 non-radioactive cytotoxicity detection kit. Using conventional molecular biology techniques, the mouse GPC3 fragment (SEQ ID NO: 55) was transferred into the hepatocarcinoma cell Hepa 1-6 (Cell Collection Center of the Chinese Academy of Sciences (Shanghai)) to construct Hepa 1-6 GPC3 cells expressing the mouse GPC3 protein.

The number of target cells is (Hepa 1-6:10000/well; Hepa 1-6 GPC3:10000/well), at an effector target ratio of 1:1, 1:3, co-cultured with effector cells for 18 h for detection (1640+10% FBS, 200 ul of system), the effector cells are T cells expressing TCR fusion protein, GPC3-SL-mCD3ε cells, GPC3-SL-CD3ε-F2A-mCCL21b cells, GPC3-SL-mCD3ε-F2A-mIL7-P2A-mCCL21b cells, GPC3-SL-mCD3ε-NFAT-mIL12 cells, GPC3-SL-mCD3ε-F2A-mRunx3 cells, and Untransduced (UTD) T cells.

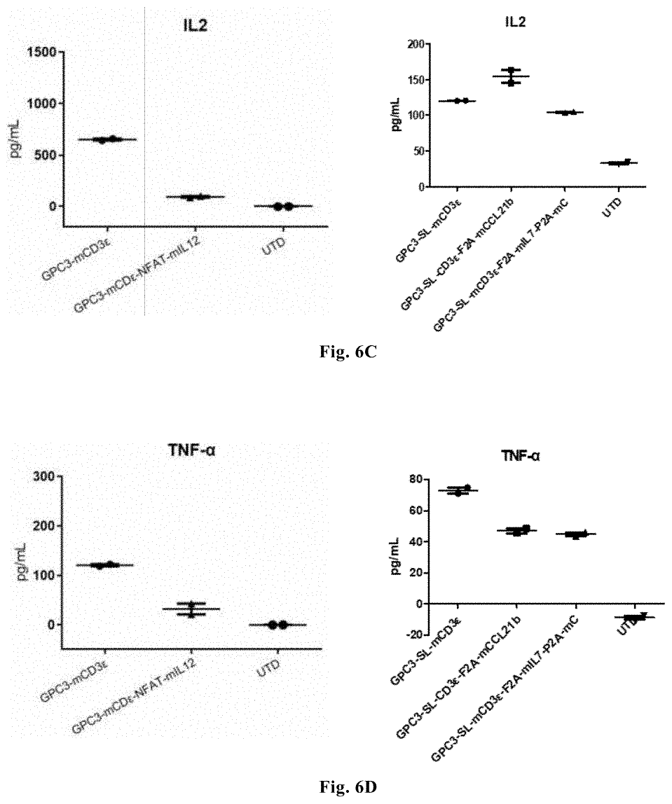

The experimental results are shown in FIG. 5. For Hepa 1-6 cells not expressing GPC3, the above-mentioned T cells expressing the TCR fusion protein have no cytotoxic killing effects under different effector target ratios. For Hepa 1-6 GPC3 cells expressing the target antigen GPC3, all of GPC3-SL-mCD3ε cells, GPC3-SL-CD3ε-F2A-mCCL21b cells, GPC3-SL-mCD3ε-F2A-mIL7-P2A-mCCL21b cells, GPC3-SL-mCD3ε-NFAT-mIL12 cells and GPC3-SL-mCD3ε-F2A-mRunx3 cells can achieve better killing effects, showing very significant specific cytotoxicities, and a gradient-dependency on effector target ratio, that is, the higher effector target ratio, the stronger the cytotoxicity; in which, when the effector target ratio is 1:1, the cytotoxicities to Hepa 1-6 GPC3 were 70.7%, 73.2%, 73.4%, 91.5%, 82.2%, respectively.

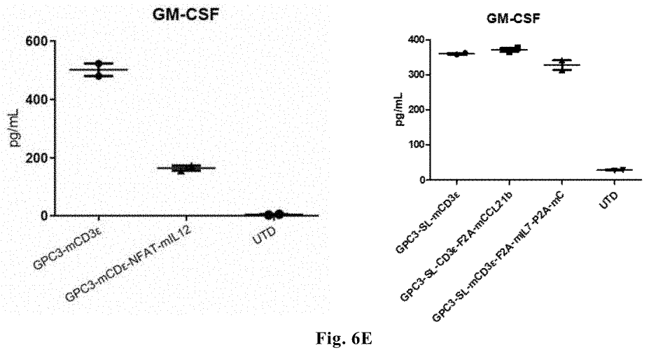

ELISA was used to detect the cytokine secretion of T cells expressing TCR fusion protein after co-incubated with hepatocarcinoma Hepa 1-6 GPC3 at an effector target ratio of 1:1 for 24 hours. The expression of cytokine in the cell culture supernatant was detected. The secretion of IFN-γ, Granzyme-B, IL2, TNF-α and GM-CSF are shown in FIGS. 6A, 6B, 6C, 6D, and 6E, respectively, in which Granzyme-B represents T cell degranulation, and GM-CSF is a cytokine released after T cell activation.

When co-incubated with target cells Hepa 1-6 GPC3 overexpressing GPC3, T cells expressing TCR fusion protein secreted a large amount of IFN-γ and Granzyme-B, among which GPC3-SL-mCD3ε-NFAT-mIL12 secreted a higher amount than that in other groups.

### Example 7. Anti-Tumor Treatment Experiment of Subcutaneous Xenograft Tumor

1. Subcutaneous Xenograft Tumor of Hepa 1-6 GPC3 Liver Cancer Cells in C57BL/6 Mice

1×10⁷ of hepatocarcinoma cells Hepa 1-6 GPC3 were subcutaneously inoculated into the right axilla of female C57BL/6 mice (Shanghai Xipuer-Bikai Experimental Animal Co., Ltd.), and the inoculation day was recorded as D0.

At D7 after subcutaneous inoculation of tumor tissue, the average tumor volume was about 355-373 mm³. T cells expressing TCR fusion protein were injected into the tail vein at a dosage of 1.5×10⁶ cells/animal. In the blank control group, 1.5×10⁶ cells per animal was injected.

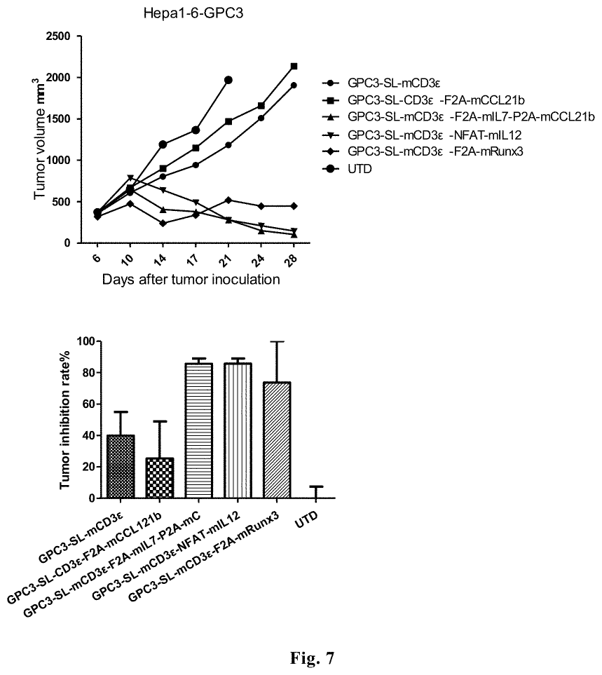

After administration of T cells expressing the TCR fusion protein, the volume of Hepa 1-6 GPC3 xenograft tumor was measured every 3-4 days, and the changes in tumor volume of each group of mice were recorded. The calculation formula for tumor volume is: (length×width²)/2. The results are shown in FIG. 7. 21 days after tumor inoculation, compared with UTD group, the tumor inhibition rates of GPC3-SL-mCD3ε, GPC3-SL-CD3ε-F2A-mCCL21b, GPC3-SL-mCD3ε-F2A-mIL7-P2A-mCCL21b, GPC3-SL-mCD3ε-NFAT-mIL12 and GPC3-SL-mCD3ε-F2A-mRunx3 treatment groups were 39.9%, 25.3%, 85.75%, 85.8% and 73.7%, respectively.

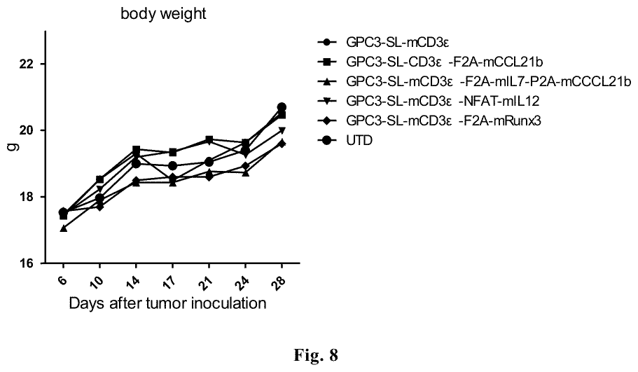

There is no significant changes in the body weight of the mice, as compared with the control group, and the results are shown in FIG. 8.

The following table lists the sequences involved herein:

| SEQ | | |
|---|---|---|
| ID | | |
| NO: | Name | Sequence |
| 1 | claudin18.2- | Qvqlqesgpglikpsqtlsltctvsggsissgynwhwirqppgkglewigyihytgstnynpalrsrvtisvdts |
| | scFv | knqfslklssvtaadtaiyycariyngnsfpywgqgttvtvssggggsggggsggggsdivMtqspdslayslge |
| | | ratinckssqslfnsgnqknyltwyqqkpgqppklliywastresgvpdrfsgsgsgtdftltisslqaedvavy |
| | | ycqnaysfpytfgggtkleikr |
| 2 | long linker | aaievMypppylggggsggggsggggsle |
| 3 | CD3ε | dgneeMggitqtpykvsisgttviltcpqypgseilwqhndkniggdeddknigsdedhlslkefseleqsgyyv |
| | | cyprgskpedanfylylrarvcencMeMdvMsvativivdicitggllllvyywsknrkakakpvtrgagaggrqr |
| | | gqnkerpppvpnpdyepirkgqrdlysglnqrri |
| 4 | claudin18.2- | Caggtgcagctgcaggagagcggccccggcctgatcaagcccagccagaccctgagcctgacctgcaccgtgagc |
| | scFv | Ggcggcagcatcagcagcggctacaactggcactggatccggcagccccccggcaagggcctggagtggatcggc |
| | | Tacatccactacaccggcagcaccaactacaaccccgccctgcggagccgggtgaccatcagcgtggacaccagc |
| | | Aagaaccagttcagcctgaagctgagcagcgtgaccgccgccgacaccgccatctactactgcgcccggatctac |
| | | Aacggcaacagcttcccctactggggccagggcaccaccgtgaccgtgagcagcggtggaggcggttcaggcgga |
| | | Ggtggttctggcggtggcggatcggacatcgtgatgacccagagccccgacagcctggccgtgagcctgggcgag |
| | | Cgggccaccatcaactgcaagagcagccagagcctgttcaacagcggcaaccagaagaactacctgacctggtac |
| | | Cagcagaagcccggccagccccccaagctgctgatctactgggccagcacccgggagagcggcgtgcccgaccgg |
| | | Ttcagcggcagcggcagcggcaccgacttcaccctgaccatcagcagcctgcaggccgaggacgtggccgtgtac |
| | | tactgccagaacgcctacagcttcccctacaccttcggcggcggcaccaagctggagatcaagcgg |
| 5 | long linker | Gcggccgcaattgaagttatgtatcctcctccttacctaggtggcggcggttctggtggcggcggttctggtggc |
| | | ggcggttctctcgag |
| 6 | CD3ε | Gatggtaatgaagaaatgggtggtattacacagacaccatataaagtctccatctctggaaccacagtaatattg |
| | | Acatgccctcagtatcctggatctgaaatactatggcaacacaatgataaaaacataggcggtgatgaggatgat |
| | | Aaaaacataggcagtgatgaggatcacctgtcactgaaggaattncagaattggagcaaagtggttattatgtc |
| | | Tgctaccccagaggaagcaaaccagaagatgcgaacttttatctctacctgagggcaagagtgtgtgagaactgc |
| | | Atggagatggatgtgatgtcggtggccacaattgtcatagtggacatctgcatcactgggggcttgctgctgctg |
| | | Gtttactactggagcaagaatagaaaggccaaggccaagcctgtgacacgaggagcgggtgctggcggcaggcaa |
| | | Aggggacaaaacaaggagaggccaccacctgttcccaacccagactatgagcccatccggaaaggccagcgggac |
| | | ctgtattctggcctgaatcagagacgcatc |
| 7 | claudin18.2- | Caggtgcagctgcaggagagcggccccggcctgatcaagcccagccagaccctgagcctgacctgcaccgtgagc |
| | LL-CD3ε | Ggcggcagcatcagcagcggctacaactggcactggatccggcagccccccggcaagggcctggagtggatcggc |
| | | Tacatccactacaccggcagcaccaactacaaccccgccctgcggagccgggtgaccatcagcgtggacaccagc |
| | | Aagaaccagttcagcctgaagctgagcagcgtgaccgccgccgacaccgccatctactactgcgcccggatctac |
| | | Aacggcaacagcttcccctactggggccagggcaccaccgtgaccgtgagcagcggtggaggcggttcaggcgga |
| | | Ggtggttctggcggtggcggatcggacatcgtgatgacccagagccccgacagcctggccgtgagcctgggcgag |
| | | Cgggccaccatcaactgcaagagcagccagagcctgttcaacagcggcaaccagaagaactacctgacctggtac |
| | | Cagcagaagcccggccagccccccaagctgctgatctactgggccagcacccgggagagcggcgtgcccgaccgg |
| | | Ttcagcggcagcggcagcggcaccgacttcaccctgaccatcagcagcctgcaggccgaggacgtggccgtgtac |
| | | Tactgccagaacgcctacagcttcccctacaccttcggcggcggcaccaagctggagatcaagcgggcggccgca |
| | | Attgaagttatgtatcctcctccttacctaggtggcggcggttctggtggcggcggttctggtggcggcggttct |
| | | Ctcgaggatggtaatgaagaaatgggtggtattacacagacaccatataaagtctccatctctggaaccacagta |
| | | Atattgacatgccctcagtatcctggatctgaaatactatggcaacacaatgataaaaacataggcggtgatgag |
| | | Gatgataaaaacataggcagtgatgaggatcacctgtcactgaaggaanttcagaattggagcaaagtggttat |
| | | Tatgtctgctaccccagaggaagcaaaccagaagatgcgaacttttatctctacctgagggcaagagtgtgtgag |
| | | Aactgcatggagatggatgtgatgtcggtggccacaattgtcatagtggacatctgcatcactgggggcttgctg |
| | | Ctgctggtttactactggagcaagaatagaaaggccaaggccaagcctgtgacacgaggagcgggtgctggcggc |
| | | Aggcaaaggggacaaaacaaggagaggccaccacctgttcccaacccagactatgagcccatccggaaaggccag |
| | | cgggacctgtattctggcctgaatcagagacgcatc |
| 8 | CD3 γ | qsikgnhlykyydyqedgsylltcdaeaknitwfkdglcMigfltedkkkwnlgsnakdprgMyqckgsqnkskpl |
| | | qvyyrMcqncielnaatisgflfaeivsifvlavgvyfiagqdgyrqsrasdkqtllpndqlyqplkdreddqys |
| | | hlqgnqlrrn |
| 9 | CD3 γ | Cagtcaatcaaaggaaaccacttggttaaggtgtatgactatcaagaagatggttcggtacttctgacttgtga |
| | | Tgcagaagccaaaaatatcacatggtttaaagatgggaagatgatcggcttcctaactgaagataaaaaaaaat |
| | | Ggaatctgggaagtaatgccaaggacccacgagggatgtatcagtgtaaaggatcacagaacaagtcaaaacca |
| | | Ctccaagtgtattacagaatgtgtcagaactgcattgaactaaatgcagccaccatatctggctttctctttgc |
| | | Tgaaatcgtcagcattttcgtccttgctgttggggtctacttcattgctggacaggatggagttcgccagtcga |
| | | Gagcttcagacaagcagactctgttgcccaatgaccagctctaccagcccctcaaggatcgagaagatgaccag |
| | | tacagccaccttcaaggaaaccagttgaggaggaat |
| 10 | claudin18.2- | Caggtgcagctgcaggagagcggccccggcctgatcaagcccagccagaccctgagcctgacctgcaccgt |
| | LL-CD3 γ | Gagcggcggcagcatcagcagcggctacaactggcactggatccggcagccccccggcaagggcctggagt |
| | | Ggatcggctacatccactacaccggcagcaccaactacaaccccgccctgcggagccgggtgaccatcagc |
| | | Gtggacaccagcaagaaccagttcagcctgaagctgagcagcgtgaccgccgccgacaccgccatctacta |
| | | Ctgcgcccggatctacaacggcaacagcttcccctactggggccagggcaccaccgtgaccgtgagcagcg |
| | | Gtggaggcggttcaggcggaggtggttctggcggtggcggatcggacatcgtgatgacccagagccccgac |
| | | Agcctggccgtgagcctgggcgagcgggccaccatcaactgcaagagcagccagagcctgttcaacagcgg |
| | | Caaccagaagaactacctgacctggtaccagcagaagcccggccagccccccaagctgctgatctactggg |
| | | Ccagcacccgggagagcggcgtgcccgaccggttcagcggcagcggcagcggcaccgacttcaccctgacc |
| | | Atcagcagcctgcaggccgaggacgtggccgtgtactactgccagaacgcctacagcttcccctacacctt |
| | | Cggcggcggcaccaagctggagatcaagcgggcggccgcaattgaagttatgtatcctcctccttacctag |
| | | Gtggcggcggttctggtggcggcggttctggtggcggcggttctctcgagcagtcaatcaaaggaaaccac |
| | | Ttggttaaggtgtatgactatcaagaagatggttcggtacttctgacttgtgatgcagaagccaaaaatat |
| | | Cacatggtttaaagatgggaagatgatcggatcctaactgaagataaaaaaaaatggaatctgggaagta |
| | | Atgccaaggacccacgagggatgtatcagtgtaaaggatcacagaacaagtcaaaaccactccaagtgtat |
| | | Tacagaatgtgtcagaactgcattgaactaaatgcagccaccatatctggctttctctttgctgaaatcgt |
| | | Cagcattttcgtccttgctgttggggtctacttcattgctggacaggatggagttcgccagtcgagagctt |
| | | Cagacaagcagactctgttgcccaatgaccagctctaccagcccctcaaggatcgagaagatgaccagtac |
| | | agccaccttcaaggaaaccagttgaggaggaat |
| 11 | short linker | aaaggggsggggsggggsle |
| | [symbol] [symbol] | |
| 12 | short linker | gcggccgcaggtggcggcggttctggtggcggcggttctggtggcggcggttctctcgag |
| 13 | claudin18.2- | Caggtgcagctgcaggagagcggccccggcctgatcaagcccagccagaccctgagcctgacctgcaccgt |
| | SL-CD3 ε | Gagcggcggcagcatcagcagcggctacaactggcactggatccggcagccccccggcaagggcctggagt |
| | | Ggatcggctacatccactacaccggcagcaccaactacaaccccgccctgcggagccgggtgaccatcagc |
| | | Gtggacaccagcaagaaccagttcagcctgaagctgagcagcgtgaccgccgccgacaccgccatctacta |
| | | Ctgcgcccggatctacaacggcaacagcttcccctactggggccagggcaccaccgtgaccgtgagcagcg |
| | | Gtggaggcggttcaggcggaggtggnctggcggtggcggatcggacatcgtgatgacccagagccccgac |
| | | Agcctggccgtgagcctgggcgagcgggccaccatcaactgcaagagcagccagagcctgttcaacagcgg |
| | | Caaccagaagaactacctgacctggtaccagcagaagcccggccagccccccaagctgctgatctactggg |
| | | Ccagcacccgggagagcggcgtgcccgaccggttcagcggcagcggcagcggcaccgacttcaccctgacc |
| | | Atcagcagcctgcaggccgaggacgtggccgtgtactactgccagaacgcctacagcttcccctacacctt |
| | | Cggcggcggcaccaagctggagatcaagcgggcggccgcaggtggcggcggttctggtggcggcggttctg |
| | | Gtggcggcggttctctcgaggatggtaatgaagaaatgggtggtattacacagacaccatataaagtctcc |
| | | Atctctggaaccacagtaatattgacatgccctcagtatcctggatctgaaatactatggcaacacaatga |
| | | Taaaaacataggcggtgatgaggatgataaaaacataggcagtgatgaggatcacctgtcactgaaggaat |
| | | Tttcagaattggagcaaagtggttattatgtctgctaccccagaggaagcaaaccagaagatgcgaacttt |
| | | Tatctctacctgagggcaagagtgtgtgagaactgcatggagatggatgtgatgtcggtggccacaattgt |
| | | Catagtggacatctgcatcactgggggcttgctgctgctggtttactactggagcaagaatagaaaggcca |
| | | Aggccaagcctgtgacacgaggagcgggtgctggcggcaggcaaaggggacaaaacaaggagaggccacca |
| | | Cctgttcccaacccagactatgagcccatccggaaaggccagcgggacctgtattctggcctgaatcagag |
| | | acgcatc |
| 14 | claudin18.2- | Caggtgcagctgcaggagagcggccccggcctgatcaagcccagccagaccctgagcctgacctgcaccgtg |
| | SL-CD3 γ | Agcggcggcagcatcagcagcggctacaactggcactggatccggcagccccccggcaagggcctggagtgg |
| | | Atcggctacatccactacaccggcagcaccaactacaaccccgccctgcggagccgggtgaccatcagcgtg |
| | | Gacaccagcaagaaccagttcagcctgaagctgagcagcgtgaccgccgccgacaccgccatctactactgc |
| | | Gcccggatctacaacggcaacagcttcccctactggggccagggcaccaccgtgaccgtgagcagcggtgga |
| | | Ggcggttcaggcggaggtggttctggcggtggcggatcggacatcgtgatgacccagagccccgacagcctg |
| | | Gccgtgagcctgggcgagcgggccaccatcaactgcaagagcagccagagcctgttcaacagcggcaaccag |
| | | Aagaactacctgacctggtaccagcagaagcccggccagccccccaagctgctgatctactgggccagcacc |
| | | Cgggagagcggcgtgcccgaccggttcagcggcagcggcagcggcaccgacttcaccctgaccatcagcagc |
| | | Ctgcaggccgaggacgtggccgtgtactactgccagaacgcctacagcttcccctacaccttcggcggcggc |
| | | Accaagctggagatcaagcgggcggccgcaggtggcggcggttctggtggcggcggttctggtggcggcggt |
| | | Tctctcgagcagtcaatcaaaggaaaccacttggttaaggtgtatgactatcaagaagatggttcggtactt |
| | | Ctgacttgtgatgcagaagccaaaaatatcacatggtttaaagatgggaagatgatcggcttcctaactgaa |
| | | Gataaaaaaaaatggaatctgggaagtaatgccaaggacccacgagggatgtatcagtgtaaaggatcacag |
| | | Aacaagtcaaaaccactccaagtgtattacagaatgtgtcagaactgcattgaactaaatgcagccaccata |
| | | Tctggctttctattgctgaaatcgtcagcattttcgtccttgctgttggggtctacttcattgctggacag |
| | | Gatggagttcgccagtcgagagcttcagacaagcagactctgttgcccaatgaccagctctaccagcccctc |
| | | aaggatcgagaagatgaccagtacagccaccttcaaggaaaccagttgaggaggaat |
| 15 | claudin18.2 | SGYNWH |
| | antibody | |
| | HCDR1 | |
| 16 | claudin18.2 | yihytgstnynpalrs |
| | antibody | |
| | HCDR2 | |
| 17 | claudin18.2 | IYNGNSFPY |
| | antibody | |
| | HCDR3 | |
| 18 | c1audin18.2 | KSSQSLFNSGNQKNYLT |
| | antibody | |
| | LCDR1 | |
| 19 | claudin18.2 | WASTRES |
| | antibody | |
| | LCDR2 | |
| 20 | claudin18.2 | QNAYSFPYT |
| | antibody | |
| | LCDR3 | |
| 21 | claudin18.2 | QVQLQESGPGLIKPSQTLSLTCTVSGGSISSGYNWHWIRQPPGKGLEWIGYIHYTG |
| | antibody VH | STNYNPALRSRVTISV |
| | | DTSKNQFSLKLSSVTAADTAIYYCARIYNGNSFPYWGQGTTVTVSS |
| 22 | claudin18.2 | divMtqspdslavslgeratinckssqslfnsgnqknyltwyqqkpgqppklliywastresgvpdrfsgsg |
| | antibody VL | sgtdftltisslqaedvavyycqnaysfpytfgggtkleikr |
| 23 | amino acid | EVQLVQSGAEVKKPGASVKVSCKASGYTFSDYEMHWVRQAPGQGLEWMGAIHP |
| | sequence of | GSGDTAYNQRFKGRVTITAD |
| | GPC3 anti- | KSTSTAYMELSSLRSEDTAVYYCARFYSYAYWGQGTLVTVSAGGGGSGGGGSG |
| | body | GGGSDIVMTQTPLSLPVTPG |
| | | EPASISCRSSQSLVHSNGNTYLQWYLQKPGQSPQLLIYKVSNRFSGVPDRFSGSGS |
| | | GTDFTLKISRVEAEDVG |
| | | VYYCSQSIYVPYTFGQGTKLEIKR |
| 24 | nucleic acid | Gaggtgcagctggtgcagagcggcgccgaggtgaagaagcccggcgccagcgtgaaggtgagctgcaaggcca |
| | sequence of | Gcggctacaccttcagcgactacgagatgcactgggtgcggcaggcccccggccagggcctggagtggatggg |
| | GPC3 anti- | Cgccatccaccccggcagcggcgacaccgcctacaaccagcggttcaagggccgggtgaccatcaccgccgac |
| | body | Aagagcaccagcaccgcctacatggagctgagcagcctgcggagcgaggacaccgccgtgtactactgcgccc |
| | | Ggttctacagctacgcctactggggccagggcaccctggtgaccgtgagcgccggtggaggcggttcaggcgg |
| | | Aggtggttctggcggtggcggatcggacatcgtgatgacccagacccccctgagcctgcccgtgacccccggc |
| | | Gagcccgccagcatcagctgccggagcagccagagcctggtgcacagcaacggcaacacctacctgcagtggt |
| | | Acctgcagaagcccggccagagcccccagctgctgatctacaaggtgagcaaccggttcagcggcgtgcccga |
| | | Ccggttcagcggcagcggcagcggcaccgacttcaccctgaagatcagccgggtggaggccgaggacgtgggc |
| | | gtgtactactgcagccagagcatctacgtgccctacaccttcggccagggcaccaagctggagatcaaacgt |
| 25 | nucleic acid | Gaggtgcagctggtgcagagcggcgccgaggtgaagaagcccggcgccagcgtgaaggtgagctgcaaggcca |
| | sequence of | Gcggctacaccttcagcgactacgagatgcactgggtgcggcaggcccccggccagggcctggagtggatggg |
| | GPC3-MCD3 ε | Cgccatccaccccggcagcggcgacaccgcctacaaccagcggttcaagggccgggtgaccatcaccgccgac |
| | | Aagagcaccagcaccgcctacatggagctgagcagcctgcggagcgaggacaccgccgtgtactactgcgccc |
| | | Ggttctacagctacgcctactggggccagggcaccctggtgaccgtgagcgccggtggaggcggttcaggcgg |
| | | Aggtggttctggcggtggcggatcggacatcgtgatgacccagacccccctgagcctgcccgtgacccccggc |
| | | Gagcccgccagcatcagctgccggagcagccagagcctggtgcacagcaacggcaacacctacctgcagtggt |
| | | Acctgcagaagcccggccagagcccccagctgctgatctacaaggtgagcaaccggttcagcggcgtgcccga |
| | | Ccggttcagcggcagcggcagcggcaccgacttcaccctgaagatcagccgggtggaggccgaggacgtgggc |
| | | Gtgtactactgcagccagagcatctacgtgccctacaccttcggccagggcaccaagctggagatcaaacgtg |
| | | Cggccgcaggtggcggcggttctggtggcggcggttctggtggcggcggttctctcgagatgcggtggaacac |
| | | Tttctggggcatcctgtgcctcagcctcctagctgttggcacttgccaggacgatgccgagaacattgaatac |
| | | Aaagtctccatctcaggaaccagtgtagagttgacgtgccctctagacagtgacgagaacttaaaatgggaaa |
| | | Aaaatggccaagagctgcctcagaagcatgataagcacctggtgctccaggatttctcggaagtcgaggacag |
| | | Tggctactacgtctgctacacaccagcctcaaataaaaacacgtacttgtacctgaaagctcgagtgtgtgag |
| | | Tactgtgtggaggtggacctgacagcagtagccataatcatcattgttgacatctgtatcactctgggcttgc |
| | | Tgatggtcatttattactggagcaagaataggaaggccaaggccaagcctgtgacccgaggaaccggtgctgg |
| | | Tagcaggcccagagggcaaaacaaggagcggccaccacctgttcccaacccagactatgagcccatccgcaaa |
| | | ggccagcgggacctgtattctggcctgaatcagagagcagtc |
| 26 | GPC3 anti- | DYEMH |
| | body HCDR1 | |
| 27 | GPC3 anti | AIHPGSGDTAYNQRFKG |
| | body HCDR2 | |
| 28 | GPC3 anti- | FYSYAY |
| | body HCDR3 | |
| 29 | GPC3 anti- | RSSQSLVHSNGNTYLQ |
| | body LCDR1 | |
| 30 | GPC3 anti- | KVSNRFS |
| | body LCDR2 | |
| 31 | GPC3 anti- | SQSIYVPYT |
| | body LCDR3 | |
| 32 | GPC3 anti- | EVQLVQSGAEVKKPGASVKVSCKASGYTFSDYEMHWVRQAPGQGLEWMGAIHP |
| | body VH | GSGDTAYNQRFKGRVTITA |
| | | DKSTSTAYMELSSLRSEDTAVYYCARFYSYAYWGQGTLVTVSA |
| 33 | GPC3 anti- | DIVMTQTPLSLPVTPGEPASISCRSSQSLVHSNGNTYLQWYLQKPGQSPQLLIYKV |
| | body VL | SNRFSGVPDRFSGSGS |
| | | GTDFTLKISRVEAEDVGVYYCSQSIYVPYTFGQGTKLEIKR |
| 34 | mCD3 ε | MRWNTFWGILCLSLLAVGTCQDDAENIEYKVSISGTSVELTCPLDSDENLKWEKN |
| | | GQELPQKHDKHLVLQDF |
| | | SEVEDSGYVVCYTPASNKNTYLYLKARVCEYCVEVDLTAVAIIIIVDICITLGLLM |
| | | VIYYWSKNRKAKAKPV |
| | | TRGTGAGSRPRGQNKERPPPVPNPDYEPIRKGQRDLYSGLNQRAV |
| 35 | mCD3 ε | Atgcggtggaacactttctggggcatcctgtgcctcagcctcctagctgttggcacttgccaggacgatgcc |
| | | Gagaacattgaatacaaagtctccatctcaggaaccagtgtagagttgacgtgccctctagacagtgacgag |
| | | Aacttaaaatgggaaaaaaatggccaagagctgcctcagaagcatgataagcacctggtgctccaggatttc |
| | | Tcggaagtcgaggacagtggctactacgtctgctacacaccagcctcaaataaaaacacgtacttgtacctg |
| | | Aaagctcgagtgtgtgagtactgtgtggaggtggacctgacagcagtagccataatcatcattgttgacatc |
| | | Tgtatcactctgggcttgctgatggtcatttattactggagcaagaataggaaggccaaggccaagcctgtg |
| | | Acccgaggaaccggtgctggtagcaggcccagagggcaaaacaaggagcggccaccacctgttcccaaccca |
| | | gactatgagcccatccgcaaaggccagcgggacctgtattctggcctgaatcagagagcagtc |
| 36 | F2A | VKQTLNFDLLKLAGDVESNPGP |
| 37 | F2A | gtgaaacagactttgaattttgaccttctgaagttggcaggagacgttgagtccaaccctgggccc |
| 38 | mCCL21b | MAQMMTLSLLSLVLALCIPWTQGSDGGGQDCCLKYSQKKIPYSIVRGYRKQEPSL |
| | | GCPIPAILFLPRKHSK |
| | | PELCANPEEGWVQNLMRRLDQPPAPGKQSPGCRKNRGTSKSGKKGKGSKGCKRT |
| | | EQTQPSRG |
| 39 | mCCL21b | Atggctcagatgatgactctgagcctccttagcctggtcctggctctctgcatcccctggacccaaggcag |
| | | Tgatggagggggacaggactgctgccttaagtacagccagaagaaaattccctacagtattgtccgaggct |
| | | Ataggaagcaagaaccaagtttaggctgtcccatcccggcaatcctgttcttaccccggaagcactctaag |
| | | Cctgagctatgtgcaaaccctgaggaaggctgggtgcagaacctgatgcgccgcctggaccagcctccagc |
| | | Cccagggaaacaaagccccggctgcaggaagaaccggggaacctctaagtctggaaagaaaggaaagggct |
| | | ccaagggctgcaagagaactgaacagacacagccctcaagagga |
| 40 | MIL7 | MFHVSFRYIFGIPPLILVLLPVTSSECHIKDKEGKAYESVLMISIDELDKMTGTDSN |
| | | CPNNEPNFFRKHVC |
| | | DDTKEAAFLNRAARKLKQFLKMNISEEFNVHLLTVSQGTQTLVNCTSKEEKNVK |
| | | EQKKNDACFLKRLLREI |
| | | KTCWNKILKGSI |
| 41 | MIL7 | Atgttccatgtttatttagatatatctttggaattcctccactgatccttgttctgctgcctgtcacat |
| | | Catctgagtgccacattaaagacaaagaaggtaaagcatatgagagtgtactgatgatcagcatcgatga |
| | | Attggacaaaatgacaggaactgatagtaattgcccgaataatgaaccaaactnntagaaaacatgta |
| | | Tgtgatgatacaaaggaagctgatttctaaatcgtgctgctcgcaagttgaagcaatttcttaaaatga |
| | | Atatcagtgaagaattcaatgtccacttactaacagtatcacaaggcacacaaacactggtgaactgcac |
| | | Aagtaaggaagaaaaaaacgtaaaggaacagaaaaagaatgatgcatgtttcctaaagagactactgaga |
| | | gaaataaaaacttgttggaataaaattttgaagggcagtata |
| 42 | P2A | ATNFSLLKQAGDVEENPGP |
| 43 | P2A | gctactaacttcagcctgctgaagcaggctggagacgtggaggagaaccctggacct |
| 44 | NFAT | KRKICFIQKALRGKFVSYRRR-EENLFHTEGVKRKICFIQKALRGKFVSYRRR-EEN |
| | | LFHTEGV |
| 45 | NFAT | AAGAGGAAAATTTGTTTCATACAGAAGGCGTTAAGAGGAAAATTTGTTTCATA |
| | | CAGAAGGCGTTAAGAG |
| | | GAAAATTTGTTTCATACAGAAGGCGTTAAGAGGAAAATTTGTTTCATACAGAA |
| | | GGCGTTAAGAGGAAAA |
| | | TTTGTTTCATACAGAAGGCGTTAAGAGGAAAATTTGTTTCATACAGAAGGCGT |
| | | T |
| 46 | mIL2 Minimal | NIVTPPYYFSSINSINCLPC-RAAYHPC-SLLTVTSSP |
| | promoter | |
| 47 | mIL2 Minimal | AACATCGTGACACCCCCATATTATTTTTCCAGCATTAACAGTATAAATTGCCTC |
| | promoter | CCATGCTGAAGAGCTGCCT |
| | | ATCACCCTTGCTAATCACTCCTCACAGTGACCTCAAGTCCT |
| 48 | mIL12 | MWELEKDVYVVEVDWTPDAPGETVNLTCDTPEEDDITWTSDQRHGVIGSGKTLT |
| | | ITVKEFLDAGQYTCHKGGET |
| | | LSHSHLLLHKKENGIWSTEILKNFKNKTFLKCEAPNYSGRFTCSWLVQRNMDLKF |
| | | NIKSSSSSPDSRAVTCGMA |
| | | SLSAEKVTLDQRDYEKYSVSCQEDVTCPTAEETLPIELALEARQQNKYENYSTSFF |
| | | IRDIIKPDPPKNLQMKPL |
| | | KNSQVEVSWEYPDSWSTPHSYFSLKFFVRIQRKKEKMKETEEGCNQKGAFLVEK |
| | | TSTEVQCKGGNVCVQAQDRY |
| | | YNSSCSKWACVPCRVRSGGGGSGGGGSGGGGSCQSRYLLFLATLALLNHLSLAR |
| | | VIPVSGPARCLSQSRNLLKT |
| | | TDDMVKTAREKLKHYSCTAEDIDHEDITRDQTSTLKTCLPLELHKNESCLATRETS |
| | | STTRGSCLPPQKTSLMMT |
| | | LCLGSIYEDLKMYQTEFQAINAALQNHNHQQIILDKGMLVAIDELMQSLNHNGET |
| | | LRQKPPVGEADPYRVKMKL |
| | | CILLHAFSTRVVTINRVMGYLSSA |
| 49 | mIL12 | Atgtgggagctggagaaagacgtttatgttgtagaggtggactggactcccgatgcccctggagaaacagtgaacc |
| | | Tcacctgtgacacgcctgaagaagatgacatcacctggacctcagaccagagacatggagtcataggctctggaaa |
| | | Gaccctgaccatcactgtcaaagagtttctagatgctggccagtacacctgccacaaaggaggcgagactctgagc |
| | | Cactcacatctgctgctccacaagaaggaaaatggaatttggtccactgaaattttaaaaaatttcaaaaacaaga |
| | | Ctttcctgaagtgtgaagcaccaaattactccggacggttcacgtgctcatggctggtgcaaagaaacatggactt |
| | | Gaagttcaacatcaagagcagtagcagttcccctgactctcgggcagtgacatgtggaatggcgtctctgtctgca |
| | | Gagaaggtcacactggaccaaagggactatgagaagtattcagtgtcctgccaggaggatgtcacctgcccaactg |
| | | Ccgaggagaccctgcccattgaactggcgttggaagcacggcagcagaataaatatgagaactacagcaccagctt |
| | | Cttcatcagggacatcatcaaaccagacccgcccaagaacttgcagatgaagcctttgaagaactcacaggtggag |
| | | Gtcagctgggagtaccctgactcctggagcactccccattcctacttctccctcaagttctttgttcgaatccagc |
| | | Gcaagaaagaaaagatgaaggagacagaggaggggtgtaaccagaaaggtgcgttcctcgtagagaagacatctac |
| | | Cgaagtccaatgcaaaggcgggaatgtctgcgtgcaagctcaggatcgctattacaattcctcatgcagcaagtgg |
| | | Gcatgtgttccctgcagggtccgatccggtggcggtggctcgggcggtggtgggtcgggtggcggcggatcttgtc |
| | | Aatcacgctacctcctctttttggccacccttgccctcctaaaccacctcagtttggccagggtcattccagtctc |
| | | Tggacctgccaggtgtcttagccagtcccgaaacctgctgaagaccacagatgacatggtgaagacggccagagaa |
| | | Aaactgaaacattattcctgcactgctgaagacatcgatcatgaagacatcacacgggaccaaaccagcacattga |
| | | Agacctgtttaccactggaactacacaagaacgagagttgcctggctactagagagacttcttccacaacaagagg |
| | | Gagctgcctgcccccacagaagacgtctttgatgatgaccctgtgccttggtagcatctatgaggacttgaagatg |
| | | Taccagacagagttccaggccatcaacgcagcacttcagaatcacaaccatcagcagatcattctagacaagggca |
| | | Tgctggtggccatcgatgagctgatgcagtctctgaatcataatggcgagactctgcgccagaaacctcctgtggg |
| | | Agaagcagacccttacagagtgaaaatgaagctctgcatcctgcttcacgccttcagcacccgcgtcgtgaccatc |
| | | aacagggtgatgggctatctgagctccgcc |
| 50 | mRunx3 | MRIPVDPSTSRRFTPPSTAFPCGGGGGGKMGENSGALSAQATAGPGGRTRPEVRS |
| | | MVDVLADHAGELVRTDSPNF |
| | | LCSVLPSHWRCNKTLPVAFKVVALGDVPDGTVVTVMAGNDENYSAELRNASAV |
| | | MKNQVARFNDLRFVGRSGRGKS |
| | | FTLTITVFTNPTQVATYHRAIKVTVDGPREPRRHRQKIEDQTKAFPDRFGDLRMRV |
| | | TPSTPSPRGSLSTTSHFSS |
| | | QAQTPIQGSSDLNPFSDPRQFDRSFPTLQSLTESRFPDPRMHYPGAMSAAFPYSATP |
| | | SGTSLGSLSVAGMPASSR |
| | | FHHTYLPPPYPGAPQSQSGPFQANPAPYHLFYGASSGSYQFSMAAAGGGERSPTR |
| | | MLTSCPSGASVSAGNLMNPS |
| | | LGQADGVEADGSHSNSPTALSTPGRMDEAVWRPY |
| 51 | mRunx3 | Atgcgtattcccgtagacccgagcaccagccgccgcttcactcccccctccacggccttcccctgcggcggcggc |
| | | Ggcggcggcaagatgggcgagaacagcggcgcgctaagcgcgcaggcaaccgcgggccccggcggccgcacccgg |
| | | Cccgaagtgcgctcgatggtggacgtgctggccgaccacgcgggagagctcgtgcgcaccgacagccccaacttc |
| | | Ctctgctccgtgctgccctcgcactggcgctgcaacaagacgctgccggtcgccttcaaggtggtggccctgggg |
| | | Gatgtgccggatggaacggtggtgaccgtgatggccggcaatgatgagaactactccgccgagctgcgcaacgct |
| | | Tccgctgtcatgaagaaccaagtggccaggttcaacgaccttcgattcgtgggccgcagtgggcgagggaagagt |
| | | Ttcacgctcacaatcaccgtgttcaccaaccctacccaagtggctacctaccaccgagccatcaaggtcactgtg |
| | | Gatggaccccgggaaccccgacggcaccggcagaagatagaagaccagaccaaggccttccccgaccgctttgga |
| | | Gacctgcgcatgcgtgtaacaccaagcacacccagcccccgtggctctctcagcaccacgagccacttcagcagc |
| | | Caggcccagaccccaatccaaggctcctcagacctgaaccccttctccgacccccgccagtttgaccgctccttc |
| | | Cctacgctgcagagcctcacagagagccgcttcccggaccccaggatgcactacccgggagccatgtctgccgcc |
| | | Ttcccctacagcgccacaccatcgggcaccagcctgggcagcctgagcgtggcgggcatgccggccagcagccgc |
| | | Ttccaccacacctacctccctccgccctaccccggggccccacagagccagagcgggccctttcaggccaacccc |
| | | Gcgccctaccacctcttttacggcgcctcctccggctcctaccagttctccatggcagccgcgggaggtggtgag |
| | | Cgctcgcccacccgcatgctgacctcctgccccagcggcgcttcggtgtcagcaggcaacctcatgaaccccagc |
| | | Ctgggccaggctgatggcgtggaagccgacggcagccacagcaactcgcccacggccctgagcacgccgggccgc |
| | | atggacgaggccgtgtggcggccctac |
| 52 | PA2 | NKISLFSLHLCVGFLCE |
| 53 | PA2 | aataaaatatctttattttcattacatctgtgtgttggttttttgtgtgag |
| 54 | Claudin18.2 | MAVTACQGLGFVVSLIGIAGIIAATCMDQWSTQDLYNNPVTAVFNYQGLWRSCV |
| | | RESSGFTECRGYFTLLGLPAMLQAVRALMIVGIVLGAIGLLVSIFALKCIRIGSMED |
| | | SAKANMTLTSGIMFIVSGLCAIAGVSVFANMLVTNFWMSTANMYTGMGGMVQT |
| | | VQTRYTFGAALFVGWVAGGLTLIGGVMMCIACRGLAPEETNYKAVSYHASGHS |
| | | VAYKPGGFKASTGFGSNTKNKKIYDGGARTEDEVQSYPSKHDYV |
| 55 | GPC3 | MAGTVRTACLLVAMLLGLGCLGQAQPPPPPDATCHQVRSFFQRLQPGLKWVPET |
| | | PVPGSDLQVCLPKGPTCCSRKMEEKYQLTARLNMEQLLQSASMELKFLIIQNAAV |
| | | FQEAFEIVVRHAKNYTNAMFKNNYPSLTPQAFEFVGEFFTDVSLYILGSDINVDD |
| | | MVNELFDSLFPVIYTQMMNPGLPESVLDINECLRGARRDLKVFGSFPKLIMTQVSK |
| | | SLQVTRIFLQALNLGIEVINTTDHLKFSKDCGRMLTRMWYCSYCQGLMMVKPCG |
| | | GYCNVVMQGCMAGVVEIDKYWREYILSLEELVNGMYRIYDMENVLLGLFSTIHD |
| | | SIQYVQKNGGKLTTTIGKLCAHSQQRQYRSAYYPEDLFIDKKILKVAHVEHEETLS |
| | | SRRRELIQKLKSFINFYSALPGYICSHSPVAENDTLCWNGQELVERYSQKAARNG |
| | | MKNQFNLHELKMKGPEPVVSQIIDKLKHINQLLRTMSVPKGKVLDKSLDEEGLES |
| | | GDCGDDEDECIGSSGDGMVKVKNQLRFLAELAYDLDVDDAPGNKQHGNQKDNE |
| | | ITTSHSVGNMPSPLKILISVAIYVACFFFLVH |
| 56 | claudin 18.2 | QVQLQQSGAELARPGASVKMSCKASGYTFTSYTMHWVKQRPGQGLEWIGYINPS |
| | antibody 2 | SGYTNYNQKFKDKATLTADKSSSTAYMQLSSLTSEDSAVYYCARIYYGNSFAYW |
| | | GQGTTVTVSSGGGGSGGGGSGGGGSDIVMTQSPSSLTVTAGEKVTMSCKSSQSLL |
| | | NSGNQKNYLTWYQQKPGQPPKLLIYWASTRESGVPDRFTGSGSGTDFTLTISSVQ |
| | | AEDLAVYYCQNDYSYPLTFGAGTKLELKR |
| 57 | claudin 18.2 | SYTMH |
| | antibody 2- | |
| | HCDR1 | |
| 58 | claudin 18.2 | YINPSSGYTNYNQKFKD |
| | antibody 2- | |
| | HCDR2 | |
| 59 | claudin 18.2 | IYYGNSFAY |
| | antibody 2- | |
| | HCDR3 | |
| 60 | claudin 18.2 | KSSQSLLNSGNQKNYLT |
| | antibody 2- | |
| | LCDR1 | |
| 61 | claudin 18.2 | WASTRES |
| | antibody 2- | |
| | LCDR2 | |
| 62 | claudin 18.2 | QNDYSYPLT |
| | antibody 2- | |
| | LCDR3 | |
| 63 | claudin 18.2 | QVQLQQSGAELARPGASVKMSCKASGYTFTSYTMHWVKQRPGQGLEWIGYINPS |
| | antibody 2-VH | SGYTNYNQKFKDKATLTADKSSSTAYMQLSSLTSEDSAVYYCARIYYGNSFAYW |
| | | GQGTTVTVSS |
| 64 | claudin 18.2 | DIVMTQSPSSLTVTAGEKVTMSCKSSQSLLNSGNQKNYLTWYQQKPGQPPKLLIY |
| | antibody 2-VL | WASTRESGVPDRFTGSGSGTDFTLTISSVQAEDLAVYYCQNDYSYPLTFGAGTKL |
| | | ELKR |
| 65 | claudin 18.2 | QVQLQQSGPELVKPGALVKISCKASGYTFTSYDINWVKQRPGQGLEWIGWIYPGD |
| | antibody 3 | GSTKYNEKFKGKATLTADKSSSTAYMQLSSLTSENSAVYFCARGGYRYDEAMDY |
| | | WGQGTTVTVSSGGGGSGGGGSGGGGSQIVLTQSPAIMSASPGEKVTMTCSASSSIS |
| | | YMHWYQQKPGTSPKRWIYDTSKLASGVPARFSGSGSGTSYSLTISSMEAEDAATY |
| | | YCHQRSSYPYTEGGGTKLEIKR |
| 66 | claudin 18.2 | SYDIN |
| | antibody 3- | |
| | HCDR1 | |
| 67 | claudin 18.2 | WIYPGDGSTKYNEKFKG |
| | antibody 3- | |
| | HCDR2 | |
| 68 | claudin 18.2 | |
| | antibody 3- | GGYRYDEAMDY |
| | HCDR3 | |
| 69 | claudin 18.2 | SASSSISYMH |
| | antibody 3- | |
| | LCDR1 | |
| 70 | claudin 18.2 | DTSKLAS |
| | antibody 3- | |
| | LCDR2 | |
| 71 | claudin 18.2 | HQRSSYPYT |
| | antibody 3- | |
| | LCDR3 | |
| 72 | claudin 18.2 | QVQLQQSGPELVKPGALVKISCKASGYTFTSYDINWVKQRPGQGLEWIGWIYPGD |
| | antibody 3-VH | GSTKYNEKFKGKATLTADKSSSTAYMQLSSLTSENSAVYFCARGGYRYDEAMDY |
| | | WGQGTTVTVSS |
| 73 | claudin 18.2 | QIVLTQSPAIMSASPGEKVTMTCSASSSISYMHWYQQKPGTSPKRWIYDTSKLASG |
| | antibody 3-VL | VPARFSGSGSGTSYSLTISSMEAEDAATYYCHQRSSYPYTEGGGTKLEIKR |
| 74 | claudin 18.2 | QIQLVQSGPELKKPGETVKISCKASGYTFTNYGMNWVKQAPGKGLKWMGWINT |
| | antibody 4 | NTGEPTYAREFKGRFAFSLETSASTAYLQINNLKNEDTATYFCARFSYGNSFAYW |
| | | GQGTTVTVSSGGGGSGGGGSGGGGSDIVMTQSPSSLSVSAGEKVTMSCKSSQSLL |
| | | NSGNQKNYLAWYQQKPGQPPKWYGASTRESGVPDRFTGSGSGTDFTLTISSVQ |
| | | AEDLAVYYCQNDHSYPLTFGAGTKLELKR |
| 75 | claudin 18.2 | NYGMN |
| | antibody 4- | |
| | HCDR1 | |
| 76 | claudin 18.2 | WINTNTGEPTYAEEFKG |
| | antibody 4- | |
| | HCDR2 | |
| 77 | claudin 18.2 | FSYGNSFAY |
| | antibody 4- | |
| | HCDR3 | |
| 78 | claudin 18.2 | KSSQSLLNSGNQKNYLA |
| | antibody 4- | |
| | LCDR1 | |
| 79 | claudin 18.2 | GASTRES |
| | antibody 4- | |
| | LCDR2 | |
| 80 | claudin 18.2 | QNDHSYPLT |
| | antibody 4- | |
| | LCDR3 | |
| 81 | claudin 18.2 | QIQLVQSGPELKKPGETVKISCKASGYTFTNYGMNWVKQAPGKGLKWMGWINT |
| | antibody 4-VH | NTGEPTYAEEFKGRFAFSLETSASTAYLQINNLKNEDTATYFCARFSYGNSFAYW |
| | | GQGTTVTVSS |
| 82 | claudin 18.2 | DIVMTQSPSSLSVSAGEKVTMSCKSSQSLLNSGNQKNYLAWYQQKPGQPPKLLIY |
| | antibody 4-VL | GASTRESGVPDRFTGSGSGTDFTLTISSVQAEDLAVYYCQNDHSYPLTFGAGTKLE |
| | | LKR |
| 83 | claudin 18.2 | DVQLQESGPDLVKPSQSLSLTCTVTGYSITSGYNWHWIRQFPGNKMEWMGYIHY |
| | antibody 5 | TGSTNYNPSLRSRISITRDTSKNQFFLQLNSVTTDDTATYYCTRIYNGNSFPYWGQ |
| | | GTSVTVSSGGGGSGGGGSGGGGSDIVMTQSPSSLTVTPGEKVTMTCKSSQSLENS |
| | | GNQKNYLTWYQQRPGQPPKMLIYWASTRESGVPDRFTGSGSGTDFTLTISSVQAE |
| | | DLAVFYCQNAYSFPYTEGGGTKLEIKR |
| 84 | claudin 18.2 | SGYNWH |
| | antibody 5- | |
| | HCDR1 | |
| 85 | claudin 18.2 | YIHYTGSTNYNPSLRS |
| | antibody 5- | |
| | HCDR2 | |
| 86 | claudin 18.2 | IYNGNSFPY |
| | antibody 5- | |
| | HCDR3 | |
| 87 | claudin 18.2 | KSSQSLFNSGNQKNYLT |
| | antibody 5- | |
| | LCDR1 | |
| 88 | claudin 18.2 | WASTRES |
| | antibody 5- | |
| | LCDR2 | |
| 89 | claudin 18.2 | QNAYSFPYT |
| | antibody 5- | |
| | LCDR3 | |
| 90 | claudin 18.2 | DVQLQESGPDLVKPSQSLSLTCTVTGYSITSGYNWHWIRQFPGNKMEWMGYIHY |
| | antibody 5-VH | TGSTNYNPSLRSRISITRDTSKNQFFLQLNSVTTDDTATYYCTRIYNGNSFPYWGQ |
| | | GTSVTVSS |
| 91 | claudin 18.2 | DIVMTQSPSSLTVTPGEKVTMTCKSSQSLFNSGNQKNYLTWYQQRPGQPPKMLIY |
| | antibody 5-VL | WASTRESGVPDRFTGSGSGTDFTLTISSVQAEDLAVEYCQNAYSFPYTFGGGTKLE |
| | | IKR |
| 92 | claudin 18.2 | QVQLQQSGAELARPGASVKMSCKASGYTFTSYTMHWVKQRPGQGLEWIGYIDPS |
| | antibody 6 | SGYTNYNQKFKDKATLTADKSSSTAYMQLSSLTSEDSAVYYCARIYYGNSFAYW |
| | | GQGTTVTVSSGGGGSGGGGSGGGGSDIVMTQSPSSLTVTAGEKVTMSCKSSQSLL |
| | | NSGNQKNYLTWYQQKPGQPPKLLIYWASTRESGVPDRFTGSGSGTDFTLTISSVQ |
| | | AEDLAVYYCQNDYSYPLTFGAGTKLELKR |
| 93 | claudin 18.2 | SYTMH |
| | antibody 6- | |
| | HCDR1 | |
| 94 | claudin 18.2 | YIDPSSGYTNYNQKFKD |
| | antibody 6- | |
| | HCDR2 | |
| 95 | claudin 18.2 | IYYGNSFAY |
| | antibody 6- | |
| | HCDR3 | |
| 96 | claudin 18.2 | KSSQSLLNSGNQKNYLT |
| | antibody 6- | |
| | LCDR1 | |
| 97 | claudin 18.2 | WASTRES |
| | antibody 6- | |
| | LCDR2 | |
| 98 | claudin 18.2 | QNDYSYPLT |
| | antibody 6- | |
| | LCDR3 | |
| 99 | claudin 18.2 | QVQLQQSGAELARPGASVKMSCKASGYTFTSYTMHWVKQRPGQGLEWIGYIDPS |
| | antibody 6-VH | SGYTNYNQKFKDKATLTADKSSSTAYMQLSSLTSEDSAVYYCARIYYGNSFAYW |
| | | GQGTTVTVSS |
| 100 | claudin 18.2 | DIVMTQSPSSLTVTAGEKVTMSCKSSQSLLNSGNQKNYLTWYQQKPGQPPKLLIY |
| | antibody 6-VL | WASTRESGVPDRFTGSGSGTDFTLTISSVQAEDLAVYYCQNDYSYPLTFGAGTKL |
| | | ELKR |
| 101 | claudin 18.2 | QVQLQQSGAELARPGASVKMSCKASGYTFTSYTMHWVKQRPGQGLEWIGYINP |
| | antibody 7 | ASGYTNYNQKFKDKATLTADKSSSTAYMQLSSLTSEDSAVYYCARIYYGNSFAY |
| | | WGQGTTVTVSSGGGGSGGGGSGGGGSDIVMTQSPSSLTVTAGEKVTMSCKSSQS |
| | | LLNSGNQKNYLTWYQQKPGQPPKLLIYWASTRESGVPDRFTGSGSGTDFTLTISSV |
| | | QAEDLAVYYCQNDYSYPLTFGAGTKLELKR |
| 102 | claudin 18.2 | SYTMH |
| | antibody 7- | |
| | HCDR1 | |
| 103 | claudin 18.2 | YINPASGYTNYNQKFKD |
| | antibody 7- | |
| | HCDR2 | |
| 104 | claudin 18.2 | IYYGNSFAY |
| | antibody 7- | |
| | HCDR3 | |
| 105 | claudin 18.2 | KSSQSLLNSGNQKNYLT |
| | antibody 7- | |
| | LCDR1 | |
| 106 | claudin 18.2 | WASTRES |
| | antibody 7- | |
| | LCDR2 | |
| 107 | claudin 18.2 | QNDYSYPLT |
| | antibody 7- | |
| | LCDR3 | |
| 108 | claudin 18.2 | QVQLQQSGAELARPGASVKMSCKASGYTFTSYTMHWVKQRPGQGLEWIGYINP |
| | antibody 7-VH | ASGYTNYNQKFKDKATLTADKSSSTAYMQLSSLTSEDSAVYYCARIYYGNSFAY |
| | | WGQGTTVTVSS |
| 109 | claudin 18.2 | DIVMTQSPSSLTVTAGEKVTMSCKSSQSLLNSGNQKNYLTWYQQKPGQPPKLLIY |
| | antibody 7-VL | WASTRESGVPDRFTGSGSGTDFTLTISSVQAEDLAVYYCQNDYSYPLTFGAGTKL |
| | | ELKR |
| 110 | claudin 18.2 | QVQLQQSGAELARPGASVKMSCKASGYTFTSYTMHWVKQRPGQGLEWIGYINP |
| | antibody 8 | ASGYTNYNQKFKDKATLTADKSSSTAYMQLSSLTSEDSAVYYCARIYYGNSFAY |
| | | WGQGTTVTVSSGGGGSGGGGSGGGGSDIVMTQSPSSLTVTAGEKVTMSCKSSQS |
| | | LLNSGNQKNYLTWYQQKPGQPPKLLIYWASTRESGVPDRFTGSGSGTDFTLTISSV |
| | | QAEDLAVYYCQNDYSYPLTFGAGTKLELKR |
| 111 | claudin 18.2 | SYTMH |
| | antibody 8- | |
| | HCDR1 | |
| 112 | claudin 18.2 | YINPASGYTNYNQKFKD |
| | antibody 8- | |
| | HCDR2 | |
| 113 | claudin 18.2 | IYYGNSFAY |
| | antibody 8- | |
| | HCDR3 | |
| 114 | claudin 18.2 | KSSQSLLNSGNQKNYLT |
| | antibody 8- | |
| | LCDR1 | |
| 115 | claudin 18.2 | WASTRES |
| | antibody 8- | |
| | LCDR2 | |
| 116 | claudin 18.2 | QNDYSYPLT |
| | antibody 8- | |
| | LCDR3 | |
| 117 | claudin 18.2 | QVQLQQSGAELARPGASVKMSCKASGYTFTSYTMHWVKQRPGQGLEWIGYINP |
| | antibody 8-VH | ASGYTNYNQKFKDKATLTADKSSSTAYMQLSSLTSEDSAVYYCARIYYGNSFAY |
| | | WGQGTTVTVSS |
| 118 | claudin 18.2 | DIVMTQSPSSLTVTAGEKVTMSCKSSQSLLNSGNQKNYLTWYQQKPGQPPKLLIY |
| | antibody 8-VL | WASTRESGVPDRFTGSGSGTDFTLTISSVQAEDLAVYYCQNDYSYPLTFGAGTKL |
| | | ELKR |
| 119 | claudin 18.2 | QVQLVQSGAEVKKPGASVKVSCKASGYTFTSYTMHWVRQAPGQGLEWMGYINP |
| | antibody 9 | ASGYTNYNQKFKDRVTMTRDTSTSTAYMELSSLRSEDTAVYYCARIYYGNSFAY |
| | | WGQGTLVTVSSGGGGSGGGGSGGGGSDIVMTQSPDSLAVSLGERATINCKSSQSL |
| | | LNSGNQKNYLTWYQQKPGQPPKLLIYWASTRESGVPDRFSGSGSGTDFTLTISSLQ |
| | | AEDVAVYYCQNDYSYPLTFGGGTKVEIKR |
| 120 | claudin 18.2 | SYTMH |
| | antibody 9- | |
| | HCDR1 | |
| 121 | claudin 18.2 | YINPASGYTNYNQKFKD |
| | antibody 9- | |
| | HCDR2 | |
| 122 | claudin 18.2 | IYYGNSFAY |
| | antibody 9- | |
| | HCDR3 | |
| 123 | claudin 18.2 | KSSQSLLNSGNQKNYLT |
| | antibody 9- | |
| | LCDR1 | |
| 124 | claudin 18.2 | WASTRES |
| | antibody 9- | |
| | LCDR2 | |
| 125 | claudin 18.2 | QNDYSYPLT |
| | antibody 9- | |
| | LCDR3 | |
| 126 | claudin 18.2 | QVQLVQSGAEVKKPGASVKVSCKASGYTFTSYTMHWVRQAPGQGLEWMGYINP |
| | antibody 9-VH | ASGYTNYNQKFKDRVTMTRDTSTSTAYMELSSLRSEDTAVYYCARIYYGNSFAY |
| | | WGQGTLVTVSS |
| 127 | claudin 18.2 | DIVMTQSPDSLAVSLGERATINCKSSQSLLNSGNQKNYLTWYQQKPGQPPKLLIY |
| | antibody 9-VL | WASTRESGVPDRFSGSGSGTDFTLTISSLQAEDVAVYYCQNDYSYPLTFGGGTKV |
| | | EIKR |
| 128 | claudin 18.2 | QVQLQESGPGLVKPSQTLSLTCTVSGGSISSGYNWHWIRQPPGKGLEWIGYIHYTG |
| | antibody 10 | STNYNPALRSRVTISVDTSKNQFSLKLSSVTAADTAVYYCARIYNGNSFPYWGQG |
| | | TTVTVSSGGGGSGGGGSGGGGSDIVMTQSPDSLAVSLGERATINCKSSQSLFNSGN |
| | | QKNYLTWYQQKPGQPPKLLIYWASTRESGVPDRFSGSGSGTDFTLTISSLQAEDV |
| | | AVYYCQNAYSFPYTFGGGTKLEIKR |
| 129 | claudin 18.2 | SGYNWH |
| | antibody 10- | |
| | HCDR1 | |
| 130 | claudin 18.2 | YIHYTGSTNYNPALRS |
| | antibody 10- | |
| | HCDR2 | |
| 131 | claudin 18.2 | IYNGNSFPY |
| | antibody 10- | |
| | HCDR3 | |
| 132 | claudin 18.2 | KSSQSLFNSGNQKNYLT |
| | antibody 10- | |
| | LCDR1 | |
| 133 | claudin 18.2 | WASTRES |
| | antibody 10- | |
| | LCDR2 | |
| 134 | claudin 18.2 | QNAYSFPYT |
| | antibody 10- | |
| | LCDR3 | |
| 135 | claudin 18.2 | QVQLQESGPGLVKPSQTLSLTCTVSGGSISSGYNWHWIRQPPGKGLEWIGYIHYTG |
| | antibody 10- | STNYNPALRSRVTISVDTSKNQFSLKLSSVTAADTAVYYCARIYNGNSFPYWGQG |
| | VH | TTVTVSS |
| 136 | claudin 18.2 | DIVMTQSPDSLAVSLGERATINCKSSQSLFNSGNQKNYLTWYQQKPGQPPKLLIY |
| | antibody 10- | WASTRESGVPDRFSGSGSGTDFTLTISSLQAEDVAVYYCQNAYSFPYTFGGGTKL |
| | VL | EIKR |
| 137 | GPC3 [symbol] 2 | QVQLQESGGGLVQPGRSLRLSCAASGFTFSSYAMHWVRQAPGKGLEWVSAISGS |
| | | GGSTYYADSVKGRFTISRDNSKNTLYLQMNSLRAEDTAVYYCAKDRRGSHADAF |
| | | DVWGQGTLVTVSSGGGGSGGGGSGGGGSQSALTQPPSASGSPGQSVTISCTGTSS |
| | | DVGGYNYVSWYQQYPGKAPKLLIYGNSNRPSGVPDRFSGSKSGTSASLAITGLQA |
| | | EDGADYYCQSYDSSLRVVFGGGTKVTVLG |
| 138 | GPC3 antibody | GFTFSSYAMH |
| | 2-HCDR1 | |
| 139 | GPC3 antibody | AISGSGGSTYYADSVKG |
| | 2-HCDR2 | |
| 140 | GPC3 antibody | DRRGSHADAFDV |
| | 2-HCDR3 | |
| 141 | GPC3 antibody | TGTSSDVGGYNYVS |
| | 2-LCDR1 | |
| 142 | GPC3 antibody | GNSNRPS |
| | 2-LCDR2 | |
| 143 | GPC3 antibody | QSYDSSLRVV |
| | 2-LCDR3 | |
| 144 | GPC3 antibody | QVQLQESGGGLVQPGRSLRLSCAASGFTFSSYAMHWVRQAPGKGLEWVSAISGS |
| | 2-VH | GGSTYVADSVKGRFTISRDNSKNTLYLQMNSLRAEDTAVYYCAKDRRGSHADAF |
| | | DVWGQGTLVTVSS |
| 145 | GPC3 antibody | QSALTQPPSASGSPGQSVTISCTGTSSDVGGYNYVSWYQQYPGKAPKLLIYGNSN |
| | 2-VL | RPSGVPDRFSGSKSGTSASLAITGLQAEDGADYYCQSYDSSLRVVFGGGTKVTVL |
| | | G |
| 146 | GPC3 antibody | QVQLQESGGGLVQPGRSLRLSCAASGFTFSTYAMTWVRQAPGKGLEWVSSISSSG |
| | 3 | ESTYYADSVKGRFTISRDNSKNTLYLQMNSLRAEDTAVYYCAKDRRGSHADAFD |
| | | VWGQGTLVTVSSGGGGSGGGGSGGGGSQSALTQPPSASGSPGQSVTISCTGTSSD |
| | | VGGYNYVSWYQQYPGKAPKLLIYGNSNRPSGVPDRFSGSKSGTSASLAITGLQAE |
| | | DGADYYCQSYDSSLRVVFGGGTKVTVLG |
| 147 | GPC3 antibody | GFTFSTYAMT |
| | 3-HCDR1 | |
| 148 | GPC3 antibody | SISSSGESTYYADSVKG |
| | 3-HCDR2 | |
| 149 | GPC3 antibody | DRRGSHADAFDV |
| | 3-HCDR3 | |
| 150 | GPC3 antibody | TGTSSDVGGYNYVS |
| | 3-LCDR1 | |
| 151 | GPC3 antibody | GNSNRPS |
| | 3-LCDR2 | |
| 152 | GPC3 antibody | QSYDSSLRVV |
| | 3-LCDR3 | |
| 153 | GPC3 antibody | QVQLQESGGGLVQPGRSLRLSCAASGFTFSTYAMTWVRQAPGKGLEWVSSISSSG |
| | 3-VH | ESTYVADSVKGRFTISRDNSKNTLYLQMNSLRAEDTAVYYCAKDRRGSHADAFD |
| | | VWGQGTLVTVSS |
| 154 | GPC3 antibody | QSALTQPPSASGSPGQSVTISCTGTSSDVGGYNYVSWYQQYPGKAPKLLIYGNSN |
| | 3-VL | RPSGVPDRFSGSKSGTSASLAITGLQAEDGADYYCQSYDSSLRVVFGGGTKVTVL |
| | | G |
| 155 | GPC3 antibody | QVQLQESGGGLVQPGRSLRLSCAASGFTFSTYAMAWVRQAPGKGLEWVSEISSS |
| | 4 | GSRTYVADSVKGRFTISRDNSKNTLYLQMNSLRAEDTAVYYCAKDRRGSHADAF |
| | | DVWGQGTLVTVSSGGGGSGGGGSGGGGSQSALTQPPSASGSPGQSVTISCTGTSS |
| | | DVGGYNYVSWYQQYPGKAPKLLIYGNSNRPSGVPDRFSGSKSGTSASLAITGLQA |
| | | EDGADYYCQSYDSSLRVVFGGGTKVTVLG |
| 156 | GPC3 antibody | GFTFSTYAMA |
| | 4-HCDR1 | |
| 157 | GPC3 antibody | EISSSGSRTYYADSVKG |
| | 4-HCDR2 | |
| 158 | GPC3 antibody | DRRGSHADAFDV |
| | 4-HCDR3 | |
| 159 | GPC3 antibody | TGTSSDVGGYNYVS |
| | 4-LCDR1 | |
| 160 | GPC3 antibody | GNSNRPS |
| | 4-LCDR2 | |
| 161 | GPC3 antibody | QSYDSSLRVV |
| | 4-LCDR3 | |
| 162 | GPC3 antibody | QVQLQESGGGLVQPGRSLRLSCAASGFTFSTYAMAWVRQAPGKGLEWVSEISSS |
| | 4-VH | GSRTYYADSVKGRFTISRDNSKNTLYLQMNSLRAEDTAVYYCAKDRRGSHADAF |
| | | DVWGQGTLVTVSS |
| 163 | GPC3 antibody | QSALTQPPSASGSPGQSVTISCTGTSSDVGGYNYVSWYQQYPGKAPKLLIYGNSN |
| | 4-VL | RPSGVPDRFSGSKSGTSASLAITGLQAEDGADYYCQSYDSSLRVVFGGGTKVTVL |
| | | G |
| 164 | GPC3 antibody | QVQLQESGGGLVQPGRSLRLSCAASGFTFSTYAMNWVRQAPGKGLEWVSAISMS |
| | 5 | GESTYVADSVKGRFTISRDNSKNTLYLQMNSLRAEDTAVYYCAKDRRGSHADAF |
| | | DVWGQGTLVTVSSGGGGSGGGGSGGGGSQSALTQPPSASGSPGQSVTISCTGTSS |
| | | DVGGYNYVSWYQQYPGKAPKLLIYGNSNRPSGVPDRFSGSKSGTSASLAITGLQA |
| | | EDGADYYCQSYDSSLRVVFGGGTKVTVLG |
| 165 | GPC3 antibody | GFTFSTYAMA |
| | 5-HCDR1 | |
| 166 | GPC3 antibody | AISMSGESTYYADSVKG |
| | 5-HCDR2 | |
| 167 | GPC3 antibody | DRRGSHADAFDV |
| | 5-HCDR3 | |
| 168 | GPC3 antibody | TGTSSDVGGYNYVS |
| | 5-LCDR1 | |
| 169 | GPC3 antibody | GNSNRPS |
| | 5-LCDR2 | |
| 170 | GPC3 antibody | QSYDSSLRVV |
| | 5-LCDR3 | |
| 171 | GPC3 antibody | QVQLQESGGGLVQPGRSLRLSCAASGFTFSTYAMNWVRQAPGKGLEWVSAISMS |
| | 5-VH | GESTYYADSVKGRFTISRDNSKNTLYLQMNSLRAEDTAVYYCAKDRRGSHADAF |
| | | DVWGQGTLVTVSS |
| 172 | GPC3 antibody | QSALTQPPSASGSPGQSVTISCTGTSSDVGGYNYVSWYQQYPGKAPKWYGNSN |
| | 5-VL | RPSGVPDRFSGSKSGTSASLAITGLQAEDGADYYCQSYDSSLRVVFGGGTKVTVL |
| | | G |
| 173 | GPC3 antibody | QVQLQESGGGLVQPGRSLRLSCAASGFTFSSYAMHWVRQAPGKGLEWVSAISSS |
| | 6 | GGSTYVADSVKGRFTISRDNSKNTLYLQMNSLRAEDTAVYYCAKDRRGSHADAF |
| | | DVWGQGTLVTVSSGGGGSGGGGSGGGGSQSALTQPPSASGSPGQSVTISCTGTSS |
| | | DVGHKFPVSWYQQYPGKAPKLLIYKNLLRPSGVPDRFSGSKSGTSASLAITGLQA |
| | | EDGADYYCQSYDSSLRVVFGGGTKVTVLG |
| 174 | GPC3 antibody | GFTFSSYAMH |
| | 6-HCDR1 | |
| 175 | GPC3 antibody | AISSSGGSTYYADSVKG |
| | 6-HCDR2 | |
| 176 | GPC3 antibody | DRRGSHADAFDV |
| | 6-HCDR3 | |
| 177 | GPC3 antibody | TGTSSDVGHKFPVS |
| | 6-LCDR1 | |
| 178 | GPC3 antibody | KNLLRPS |
| | 6-LCDR2 | |
| 179 | GPC3 antibody | QSYDSSLRVV |
| | 6-LCDR3 | |
| 180 | GPC3 antibody | QVQLQESGGGLVQPGRSLRLSCAASGFTFSSYAMHWVRQAPGKGLEWVSAISSS |
| | 6-VH | GGSTYYADSVKGRFTISRDNSKNTLYLQMNSLRAEDTAVYYCAKDRRGSHADAF |
| | | DVWGQGTLVTVSS |
| 181 | GPC3 antibody | QSALTQPPSASGSPGQSVTISCTGTSSDVGHKFPVSWYQQYPGKAPKLLIYKNLLR |
| | 6-VL | PSGVPDRFSGSKSGTSASLAITGLQAEDGADYYCQSYDSSLRVVFGGGTKVTVLG |
| 182 | GPC3 antibody | QVQLQESGGGLVQPGRSLRLSCAASGFTFSSYAMHWVRQAPGKGLEWVSAISSS |
| | 7 | GGSTYVADSVKGRFTISRDNSKNTLYLQMNSLRAEDTAVYYCAKDRRGSHADAF |
| | | DVWGQGTLVTVSSGGGGSGGGGSGGGGSQSALTQPPSASGSPGQSVTISCTGTSS |
| | | DVGLMHNVSWYQQYPGKAPKLLIYKSSSRPSGVPDRFSGSKSGTSASLAITGLQA |
| | | EDGADYYCQSYDSSLRVVFGGGTKVTVLG |
| 183 | GPC3 antibody | GFTFSSYAMH |
| | 7-HCDR1 | |
| 184 | GPC3 antibody | AISSSGGSTYYADSVKG |
| | 7-HCDR2 | |
| 185 | GPC3 antibody | DRRGSHADAFDV |
| | 7-HCDR3 | |
| 186 | GPC3 antibody | TGTSSDVGLMHNVS |
| | 7-LCDR1 | |
| 187 | GPC3 antibody | KSSSRPS |
| | 7-LCDR2 | |
| 188 | GPC3 antibody | QSYDSSLRVV |
| | 7-LCDR3 | |
| 189 | GPC3 antibody | QVQLQESGGGLVQPGRSLRLSCAASGFTFSSYAMHWVRQAPGKGLEWVSAISSS |
| | 7-VH | GGSTYYADSVKGRFTISRDNSKNTLYLQMNSLRAEDTAVYYCAKDRRGSHADAF |
| | | DVWGQGTLVTVSS |
| 190 | GPC3 antibody | QSALTQPPSASGSPGQSVTISCTGTSSDVGLMHNVSWYQQYPGKAPKLLIYKSSSR |
| | 7-VL | PSGVPDRFSGSKSGTSASLAITGLQAEDGADYYCQSYDSSLRVVFGGGTKVTVLG |
| 191 | GPC3 antibody | QVQLQESGGGLVQPGRSLRLSCAASGFTFSSYAMHWVRQAPGKGLEWVSAISSS |
| | 8 | GRSTYYADSVEGRFTISRDNSKNTLYLQMNSLRAEDTAVYYCAKDRRGSHADAL |
| | | NVWGQGTLVTVSSGGGGSGGGGSGGGGSQSALTQPPSASGSPGQSVTISCTGTSS |
| | | DVGGYNYVSWYQQYPGKAPKLLIYGNSNRPSGVPDRFSGSKSGTSASLAITGLQA |
| | | EDGADYYCQSYDSSLRVVFGGGTKVTVLG |
| 192 | GPC3 antibody | GFTFSSYAMH |
| | 8-HCDR1 | |
| 193 | GPC3 antibody | AISSSGRSTYYADSVEG |
| | 8-HCDR2 | |
| 194 | GPC3 antibody | DRRGSHADALNV |
| | 8-HCDR3 | |
| 195 | GPC3 antibody | TGTSSDVGGYNYVS |
| | 8-LCDR1 | |
| 196 | GPC3 antibody | KSSSRPS |
| | 8-LCDR2 | |
| 197 | GPC3 antibody | QSYDSSLRVV |
| | 8-LCDR3 | |
| 198 | GPC3 antibody | QVQLQESGGGLVQPGRSLRLSCAASGFTFSSYAMHWVRQAPGKGLEWVSAISSS |
| | 8-VH | GRSTYVADSVEGRFTISRDNSKNTLYLQMNSLRAEDTAVYYCAKDRRGSHADAL |
| | | NVWGQGTLVTVSS |
| 199 | GPC3 antibody | QSALTQPPSASGSPGQSVTISCTGTSSDVGGYNYVSWYQQYPGKAPKLLIYGNSN |
| | 8-VL | RPSGVPDRFSGSKSGTSASLAITGLQAEDGADYYCQSYDSSLRVVFGGGTKVTVL |
| | | G |
| 200 | GPC3 antibody | QVQLQQSGTELVRPGASVKLSCKALGYTFTDYEMHWVKQTPVHGLEWIGAIHPG |
| | 9 | SGDTAYNQRFKGKATLTADKSSSTAYMEYSSLTSEDSAVYYCTRFYSYAYWGQG |
| | | TLVTVSAGGGGSGGGGSGGGGSDVVMTQTPLSLPVSLGDQASISCRSSQSLVHSN |
| | | GNTYLQWYLQKPGQSPKLLIYKVSNRFSGVPDRFSGRGSGTDFTLKISRVEAEDL |
| | | GVYFCSQSIYVPYTFGGGTKLEIKR |
| 201 | GPC3 antibody | DYEMH |
| | 9-HCDR1 | |
| 202 | GPC3 antibody | AIHPGSGDTAYNQRFKG |
| | 9-HCDR2 | |
| 203 | GPC3 antibody | FYSYAY |
| | 9-HCDR3 | |
| 204 | GPC3 antibody | RSSQSLVHSNGNTYLQ |
| | 9-LCDR1 | |
| 205 | GPC3 antibody | KVSNRFS |
| | 9-LCDR2 | |
| 206 | GPC3 antibody | SQSIYVPYTF |
| | 9-LCDR3 | |
| 207 | GPC3 antibody | QVQLQQSGTELVRPGASVKLSCKALGYTFTDYEMHWVKQTPVHGLEWIGAIHPG |
| | 9-VH | SGDTAYNQRFKGKATLTADKSSSTAYMEYSSLTSEDSAVYYCTRFYSYAYWGQG |
| | | TLVTVSA |
| 208 | GPC3 antibody | DVVMTQTPLSLPVSLGDQASISCRSSQSLVHSNGNTYLQWYLQKPGQSPKLLIYK |
| | 9-VL | VSNRFSGVPDRFSGRGSGTDFTLKISRVEAEDLGVYFCSQSIYVPYTFGGGTKLEIK |
| | | R |

All documents mentioned in the present invention are cited as references in this application, as if each document was individually cited as a reference. In addition, it should be understood that after reading the above teaching content of the present invention, those skilled in the art can make various changes or modifications to the present invention, and these equivalent forms also fall within the scope defined by the appended claims of the present application.

1. A T cell receptor (TCR) fusion protein (TFP), the fusion protein comprising:
(a) a TCR subunit (or a TCR unit); and
(b) an antigen recognition unit that recognizes the antigen;
the antigen is GPC3 or claudin 18.2;
wherein the TCR subunit and the antigen recognition unit are operably connected.

2. The fusion protein of claim 1, wherein the TCR subunit comprises:
(i) at least a part of the extracellular domain of TCR, and
(ii) a TCR intracellular domain comprising a stimulatory domain derived from an intracellular signaling domain of CD3ε, CD3γ, CD3δ, TCRα, or TCRβ.

3. The fusion protein of claim 1, wherein the light chain LCDR1, LCDR2, and LCDR3 of the amino acid sequence of the antigen recognition unit that recognizes GPC3 are independently selected from or have 70-100% sequence identity with the light chain LCDR1, LCDR2, and LCDR3 shown in the following table, and/or the heavy chain HCDR1, HCDR2 and HCDR3 of the amino acid sequence of the antigen recognition unit that recognizes GPC3 are independently selected from or have 70-100% sequence identity with the heavy chain HCDR1, HCDR2 and HCDR3 shown in the following table;

| LCDR1 | LCDR2 | LCDR3 | HCDR1 | HCDR2 | HCDR3 |
|---|---|---|---|---|---|
| TGTSSDVGGYNY | GNSNR | QSYDSSL | GFTFSSYA | AISGSGGSTYYADS | DRRGSHADAF |
| VS | PS | RVV | MH | VKG | DV |
| TGTSSDVGGYNY | GNSNR | QSYDSSL | GFTFSTYA | SISSSGESTYYADSV | DRRGSHADAF |
| VS | PS | RVV | MT | KG | DV |
| TGTSSDVGGYNY | GNSNR | QSYDSSL | GFTFSTYA | EISSSGSRTYYADS | DRRGSHADAF |
| VS | PS | RVV | MA | VKG | DV |
| TGTSSDVGGYNY | GNSNR | QSYDSSL | GFTFSTYA | AISMSGESTYYADS | DRRGSHADAF |
| VS | PS | RVV | MA | VKG | DV |
| TGTSSDVGHKFP | KNLLR | QSYDSSL | GFTFSSYA | AISSSGGSTYYADS | DRRGSHADAF |
| VS | PS | RVV | MH | VKG | DV |
| TGTSSDVGLMHN | KSSSRP | QSYDSSL | GFTFSSYA | AISSSGGSTYYADS | DRRGSHADAF |
| VS | S | RVV | MH | VKG | DV |
| TGTSSDVGGYNY | KSSSRP | QSYDSSL | GFTFSSYA | AISSSGRSTYYADS | DRRGSHADAL |
| VS | S | RVV | MH | VEG | NV |
| RSSQSLVHSNGN | KVSNR | SQSIYVPY | DYEMH | AIHPGSGDTAYNQR | FYSYAY |
| TYLQ | FS | T | | FKG | |
| RSSQSLVHSNGN | KVSNR | SQSIYVPY | DYEMH | AIHPGSGDTAYNQR | FYSYAY |
| TYLQ | FS | TF | | FKG | |

or
the light chain LCDR1, LCDR2, and LCDR3 of the amino acid sequence of the antigen recognition unit that recognizes claudin 18.2 are independently selected from or have 70-100% sequence identity with the light chain LCDR1, LCDR2, and LCDR3 shown in the following table, and/or the heavy chain HCDR1, HCDR2 and HCDR3 of the amino acid sequence of the antigen recognition unit that recognizes claudin 18.2 are independently selected from or have 70-100% sequence identity with the heavy chain HCDR1, HCDR2 and HCDR3 shown in the following table;

| LCDR1 | LCDR2 | LCDR3 | HCDR1 | HCDR2 | HCDR3 |
|---|---|---|---|---|---|
| KSSQSLLNSGNQKNY | WASTR | QNDYSYP | SYTMH | YINPSSGYTNYNQKF | IYYGNSFAY |
| LT | ES | LT | | KD | |
| SASSSISYMH | DTSKL | HQRSSYP | SYDIN | WIYPGDGSTKYNEKF | GGYRYDEAM |
| | AS | YT | | KG | DY |
| KSSQSLLNSGNQKNY | GASTR | QNDHSYP | NYGM | WINTNTGEPTYAEEF | FSYGNSFAY |
| LA | ES | LT | N | KG | |
| KSSQSLFNSGNQKNY | WASTR | QNAYSFP | SGYNW | YIHYTGSTNYNPSLRS | IYNGNSFPY |
| LT | ES | YT | H | | |
| KSSQSLLNSGNQKNY | WASTR | QNDYSYP | SYTMH | YIDPSSGYTNYNQKF | IYYGNSFAY |
| LT | ES | LT | | KD | |
| KSSQSLLNSGNQKNY | WASTR | QNDYSYP | SYTMH | YINPASGYTNYNQKF | IYYGNSFAY |
| LT | ES | LT | | KD | |
| KSSQSLLNSGNQKNY | WASTR | QNDYSYP | SYTMH | YINPASGYTNYNQKF | IYYGNSFAY |
| LT | ES | LT | | KD | |
| KSSQSLLNSGNQKNY | WASTR | QNDYSYP | SYTMH | YINPASGYTNYNQKF | IYYGNSFAY |
| LT | ES | LT | | KD | |
| KSSQSLFNSGNQKNY | WASTR | QNAYSFP | SGYNW | YIHYTGSTNYNPALR | IYNGNSFPY |
| LT | ES | YT | H | S | |
| KSSQSLFNSGNQKNY | WASTR | QNAYSFP | SGYNW | YIHYTGSTNYNPALR | IYNGNSFPY. |
| LT | ES | YT | H | S | |

4. The fusion protein of claim 1, wherein the light chain variable region of the amino acid sequence of the antigen recognition unit that recognizes GPC3 is independently selected from or has 70-100% sequence identity with the light chain variable regions shown in the following table, and/or the heavy chain variable region of the amino acid sequence of the antigen recognition unit that recognizes GPC3 is independently selected from or has 70-100% sequence identity with the heavy chain variable region shown in the following table;

| VH | VL |
|---|---|
| QVQLQESGGGLVQPGRSLRLSCAASGFTFSSYAMHW | QSALTQPPSASGSPGQSVTISCTGTSSDVGGYNY |
| VRQAPGKGLEWVSAISGSGGSTYYADSVKGRFTISRD | VSWYQQYPGKAPKLLIYGNSNRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSTYAMTW | QSALTQPPSASGSPGQSVTISCTGTSSDVGGYNY |
| VRQAPGKGLEWVSSISSSGESTYYADSVKGRFTISRD | VSWYQQYPGKAPKLLIYGNSNRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSTYAMAW | QSALTQPPSASGSPGQSVTISCTGTSSDVGGYNY |
| VRQAPGKGLEWVSEISSSGSRTYYADSVKGRFTISRD | VSWYQQYPGKAPKLLIYGNSNRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSTYAMNW | QSALTQPPSASGSPGQSVTISCTGTSSDVGGYNY |
| VRQAPGKGLEWVSAISMSGESTYYADSVKGRFTISRD | VSWYQQYPGKAPKLLIYGNSNRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSSYAMHW | QSALTQPPSASGSPGQSVTISCTGTSSDVGHKFP |
| VRQAPGKGLEWVSAISSSGGSTYYADSVKGRFTISRD | VSWYQQYPGKAPKLLIYKNLLRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSSYAMHW | QSALTQPPSASGSPGQSVTISCTGTSSDVGLMHN |
| VRQAPGKGLEWVSAISSSGGSTYYADSVKGRFTISRD | VSWYQQYPGKAPKLLIYKSSSRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSSYAMHW | QSALTQPPSASGSPGQSVTISCTGTSSDVGGYNY |
| VRQAPGKGLEWVSAISSSGRSTYYADSVEGRFTISRD | VSWYQQYPGKAPKLLIYGNSNRPSGVPDRFSGS |
| NSKNTLYLQMNSLRAEDTAVYYCAKDRRGSHADAL | KSGTSASLAITGLQAEDGADYYCQSYDSSLRVV |
| NVWGQGTLVTVSS | FGGGTKVTVLG |
| EVQLVQSGAEVKKPGASVKVSCKASGYTFSDYEMH | DIVMTQTPLSLPVTPGEPASISCRSSQSLVHSNG |
| WVRQAPGQGLEWMGAIHPGSGDTAYNQRFKGRVTIT | NTYLQWYLQKPGQSPQLLIYKVSNRFSGVPDRF |
| ADKSTSTAYMELSSLRSEDTAVYYCARFYSYAYWGQ | SGSGSGTDFTLKISRVEAEDVGVYYCSQSIYVPY |
| GTLVTVSA | TFGQGTKLEIKR |
| QVQLQQSGTELVRPGASVKLSCKALGYTFTDYEMHW | DVVMTQTPLSLPVSLGDQASISCRSSQSLVHSN |
| VKQTPVHGLEWIGAIHPGSGDTAYNQRFKGKATLTA | GNTYLQWYLQKPGQSPKLLIYKVSNRFSGVPDR |
| DKSSSTAYMEYSSLTSEDSAVYYCTRFYSYAYWGQG | FSGRGSGTDFTLKISRVEAEDLGVYFCSQSIYVP |
| TLVTVSA | YTFGGGTKLEIKR |

or,
the light chain variable region of the amino acid sequence of the antigen recognition unit that recognizes claudin18.2 is independently selected from or has 70-100% sequence identity with the light chain variable regions shown in the following table, and/or the heavy chain variable region of the amino acid sequence of the antigen recognition unit that recognizes claudin18.2 is independently selected from or has 70-100% sequence identity with the heavy chain variable region shown in the following table;

| VH | VL |
|---|---|
| QVQLQQSGAELARPGASVKMSCKASGYTFTSYT | DIVMTQSPSSLTVTAGEKVTMSCKSSQSLLNS |
| MHWVKQRPGQGLEWIGYINPSSGYTNYNQKFKD | GNQKNYLTWYQQKPGQPPKLLIYWASTRESG |
| KATLTADKSSSTAYMQLSSLTSEDSAVYYCARIY | VPDRFTGSGSGTDFTLTISSVQAEDLAVYYCQ |
| YGNSFAYWGQGTTVTVSS | NDYSYPLTFGAGTKLELKR |
| QVQLQQSGPELVKPGALVKISCKASGYTFTSYDIN | QIVLTQSPAIMSASPGEKVTMTCSASSSISYMH |
| WVKQRPGQGLEWIGWIYPGDGSTKYNEKFKGKA | WYQQKPGTSPKRWIYDTSKLASGVPARFSGS |
| TLTADKSSSTAYMQLSSLTSENSAVYFCARGGYR | GSGTSYSLTISSMEAEDAATYYCHQRSSYPYT |
| YDEAMDYWGQGTTVTVSS | FGGGTKLEIKR |
| QIQLVQSGPELKKPGETVKISCKASGYTFTNYGM | DIVMTQSPSSLSVSAGEKVTMSCKSSQSLLNS |
| NWVKQAPGKGLKWMGWINTNTGEPTYAEEFKG | GNQKNYLAWYQQKPGQPPKLLIYGASTRESG |
| RFAFSLETSASTAYLQINNLKNEDTATYFCARFSY | VPDRFTGSGSGTDFTLTISSVQAEDLAVYYCQ |
| GNSFAYWGQGTTVTVSS | NDHSYPLTFGAGTKLELKR |
| DVQLQESGPDLVKPSQSLSLTCTVTGYSITSGYNW | DIVMTQSPSSLTVTPGEKVTMTCKSSQSLFNS |
| HWIRQFPGNKMEWMGYIHYTGSTNYNPSLRSRISI | GNQKNYLTWYQQRPGQPPKMLIYWASTRES |
| TRDTSKNQFFLQLNSVTTDDTATYYCTRIYNGNSF | GVPDRFTGSGSGTDFTLTISSVQAEDLAVFYC |
| PYWGQGTSVTVSS | QNAYSFPYTFGGGTKLEIKR |
| QVQLQQSGAELARPGASVKMSCKASGYTFTSYT | DIVMTQSPSSLTVTAGEKVTMSCKSSQSLLNS |
| MHWVKQRPGQGLEWIGYIDPSSGYTNYNQKFKD | GNQKNYLTWYQQKPGQPPKLLIYWASTRESG |
| KATLTADKSSSTAYMQLSSLTSEDSAVYYCARIY | VPDRFTGSGSGTDFTLTISSVQAEDLAVYYCQ |
| YGNSFAYWGQGTTVTVSS | NDYSYPLTFGAGTKLELKR |
| QVQLQQSGAELARPGASVKMSCKASGYTFTSYT | DIVMTQSPSSLTVTAGEKVTMSCKSSQSLLNS |
| MHWVKQRPGQGLEWIGYINPASGYTNYNQKFKD | GNQKNYLTWYQQKPGQPPKLLIYWASTRESG |
| KATLTADKSSSTAYMQLSSLTSEDSAVYYCARIY | VPDRFTGSGSGTDFTLTISSVQAEDLAVYYCQ |
| YGNSFAYWGQGTTVTVSS | NDYSYPLTFGAGTKLELKR |
| QVQLQQSGAELARPGASVKMSCKASGYTFTSYT | DIVMTQSPSSLTVTAGEKVTMSCKSSQSLLNS |
| MHWVKQRPGQGLEWIGYINPASGYTNYNQKFKD | GNQKNYLTWYQQKPGQPPKLLIYWASTRESG |
| KATLTADKSSSTAYMQLSSLTSEDSAVYYCARIY | VPDRFTGSGSGTDFTLTISSVQAEDLAVYYCQ |
| YGNSFAYWGQGTTVTVSS | NDYSYPLTFGAGTKLELKR |
| QVQLVQSGAEVKKPGASVKVSCKASGYTFTSYT | DIVMTQSPDSLAVSLGERATINCKSSQSLLNSG |
| MHWVRQAPGQGLEWMGYINPASGYTNYNQKFK | NQKNYLTWYQQKPGQPPKLLIYWASTRESGV |
| DRVTMTRDTSTSTAYMELSSLRSEDTAVYYCARI | PDRFSGSGSGTDFTLTISSLQAEDVAVYYCQN |
| YYGNSFAYWGQGTLVTVSS | DYSYPLTFGGGTKVEIKR |
| QVQLQESGPGLVKPSQTLSLTCTVSGGSISSGYNW | DIVMTQSPDSLAVSLGERATINCKSSQSLFNSG |
| HWIRQPPGKGLEWIGYIHYTGSTNYNPALRSRVTI | NQKNYLTWYQQKPGQPPKLLIYWASTRESGV |
| SVDTSKNQFSLKLSSVTAADTAVYYCARIYNGNS | PDRFSGSGSGTDFTLTISSLQAEDVAVYYCQN |
| FPYWGQGTTVTVSS | AYSFPYTFGGGTKLEIKR |
| QVQLQESGPGLIKPSQTLSLTCTVSGGSISSGYNW | DIVMTQSPDSLAVSLGERATINCKSSQSLFNSG |
| HWIRQPPGKGLEWIGYIHYTGSTNYNPALRSRVTI | NQKNYLTWYQQKPGQPPKLLIYWASTRESGV |
| SVDTSKNQFSLKLSSVTAADTAIYYCARIYNGNSF | PDRFSGSGSGTDFTLTISSLQAEDVAVYYCQN |
| PYWGQGTTVTVSS | AYSFPYTFGGGTKLEIKR. |

5. The fusion protein of claim 1, wherein the light chain LCDR1, LCDR2, and LCDR3 of the amino acid sequence of the antigen recognition unit that recognizes GPC3 are or have 70-100% sequence identity with a combination of the light chain LCDR1, LCDR2, and LCDR3 shown in any row of the following table, and/or the heavy chain HCDR1, HCDR2, and HCDR3 of the amino acid sequence of the antigen recognition unit that recognizes GPC3 are or have 70-100% sequence identity with a combination of the heavy chain HCDR1, HCDR2, and HCDR3 shown in any row of the following table;

| LCDR1 | LCDR2 | LCDR3 | HCDR1 | HCDR2 | HCDR3 |
|---|---|---|---|---|---|
| TGTSSDVGGYNY | GNSN | QSYDSSLR | GFTFSSYA | AISGSGGSTYYADS | DRRGSHAD |
| VS | RPS | VV | MH | VKG | AFDV |
| TGTSSDVGGYNY | GNSN | QSYDSSLR | GFTFSTYA | SISSSGESTYYADS | DRRGSHAD |
| VS | RPS | VV | MT | VKG | AFDV |
| TGTSSDVGGYNY | GNSN | QSYDSSLR | GFTFSTYA | EISSSGSRTYYADS | DRRGSHAD |
| VS | RPS | VV | MA | VKG | AFDV |
| TGTSSDVGGYNY | GNSN | QSYDSSLR | GFTFSTYA | AISMSGESTYYADS | DRRGSHAD |
| VS | RPS | VV | MA | VKG | AFDV |
| TGTSSDVGHKFP | KNLL | QSYDSSLR | GFTFSSYA | AISSSGGSTYYADS | DRRGSHAD |
| VS | RPS | VV | MH | VKG | AFDV |
| TGTSSDVGLMHN | KSSSR | QSYDSSLR | GFTFSSYA | AISSSGGSTYYADS | DRRGSHAD |
| VS | PS | VV | MH | VKG | AFDV |
| TGTSSDVGGYNY | KSSSR | QSYDSSLR | GFTFSSYA | AISSSGRSTYYADS | DRRGSHAD |
| VS | PS | VV | MH | VEG | ALNV |
| RSSQSLVHSNGN | KVSN | SQSIYVPY | DYEMH | AIHPGSGDTAYNQ | FYSYAY |
| TYLQ | RFS | T | | RFKG | |
| RSSQSLVHSNGN | KVSN | SQSIYVPY | DYEMH | AIHPGSGDTAYNQ | FYSYAY; |
| TYLQ | RFS | TF | | RFKG | |

or
the light chain LCDR1, LCDR2, and LCDR3 of the amino acid sequence of the antigen recognition unit that recognizes claudin18.2 are or have 70-100% sequence identity with a combination of the light chain LCDR1, LCDR2, and LCDR3 shown in any row of the following table, and/or the heavy chain HCDR1, HCDR2, and HCDR3 of the amino acid sequence of the antigen recognition unit that recognizes claudin18.2 are or have 70-100% sequence identity with a combination of the heavy chain HCDR1, HCDR2, and HCDR3 shown in any row of the following table;

| LCDR1 | LCDR2 | LCDR3 | HCDR1 | HCDR2 | HCDR3 |
|---|---|---|---|---|---|
| KSSQSLLNSGNQK | WASTRES | QNDYSYP | SYTMH | YINPSSGYTNYNQK | IYYGNSFAY |
| NYLT | | LT | | FKD | |
| SASSSISYMH | DTSKLAS | HQRSSYP | SYDIN | WIYPGDGSTKYNEK | GGYRYDEA |
| | | YT | | FKG | MDY |
| KSSQSLLNSGNQK | GASTRES | QNDHSYP | NYGMN | WINTNTGEPTYAEE | FSYGNSFAY |
| NYLA | | LT | | FKG | |
| KSSQSLFNSGNQK | WASTRES | QNAYSFP | SGYNWH | YIHYTGSTNYNPSL | IYNGNSFPY |
| NYLT | | YT | | RS | |
| KSSQSLLNSGNQK | WASTRES | QNDYSYP | SYTMH | YIDPSSGYTNYNQK | IYYGNSFAY |
| NYLT | | LT | | FKD | |
| KSSQSLLNSGNQK | WASTRES | QNDYSYP | SYTMH | YINPASGYTNYNQK | IYYGNSFAY |
| NYLT | | LT | | FKD | |
| KSSQSLLNSGNQK | WASTRES | QNDYSYP | SYTMH | YINPASGYTNYNQK | IYYGNSFAY |
| NYLT | | LT | | FKD | |
| KSSQSLLNSGNQK | WASTRES | QNDYSYP | SYTMH | YINPASGYTNYNQK | IYYGNSFAY |
| NYLT | | LT | | FKD | |
| KSSQSLFNSGNQK | WASTRES | QNAYSFP | SGYNWH | YIHYTGSTNYNPAL | IYNGNSFPY |
| NYLT | | YT | | RS | |
| KSSQSLFNSGNQK | WASTRES | QNAYSFP | SGYNWH | YIHYTGSTNYNPAL | IYNGNSFPY. |
| NYLT | | YT | | RS | |

6. The fusion protein of claim 1, wherein the light chain variable region and the heavy chain variable region of the amino acid sequence of the antigen recognition unit that recognizes GPC3 are or have 70-100% sequence identity with a combination of the light chain variable region and the heavy chain variable region shown in any row of the following table;

| VH | VL |
|---|---|
| QVQLQESGGGLVQPGRSLRLSCAASGFTFSSYAMHW | QSALTQPPSASGSPGQSVTISCTGTSSDVGGYNY |
| VRQAPGKGLEWVSAISGSGGSTYYADSVKGRFTISR | VSWYQQYPGKAPKLLIYGNSNRPSGVPDRFSGS |
| DNSKNTLYLQMNSLRAEDTAVYYCAKDRRGSHADA | KSGTSASLAITGLQAEDGADYYCQSYDSSLRVV |
| FDVWGQGTLVTVSS | FGGGTKVTVLG |
| QVQLQESGGGLVQPGRSLRLSCAASGFTFSTYAMTW | QSALTQPPSASGSPGQSVTISCTGTSSDVGGYNY |
| VRQAPGKGLEWVSSISSSGESTYYADSVKGRFTISRD | VSWYQQYPGKAPKLLIYGNSNRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSTYAMAW | QSALTQPPSASGSPGQSVTISCTGTSSDVGGYNY |
| VRQAPGKGLEWVSEISSSGSRTYYADSVKGRFTISRD | VSWYQQYPGKAPKLLIYGNSNRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSTYAMNW | QSALTQPPSASGSPGQSVTISCTGTSSDVGGYNY |
| VRQAPGKGLEWVSAISMSGESTYYADSVKGRFTISR | VSWYQQYPGKAPKLLIYGNSNRPSGVPDRFSGS |
| DNSKNTLYLQMNSLRAEDTAVYYCAKDRRGSHADA | KSGTSASLAITGLQAEDGADYYCQSYDSSLRVV |
| FDVWGQGTLVTVSS | FGGGTKVTVLG |
| QVQLQESGGGLVQPGRSLRLSCAASGFTFSSYAMHW | QSALTQPPSASGSPGQSVTISCTGTSSDVGHKFP |
| VRQAPGKGLEWVSAISSSGGSTYYADSVKGRFTISRD | VSWYQQYPGKAPKLLIYKNLLRPSGVPDRFSGS |
| NSKNTLYLQMNSLRAEDTAVYYCAKDRRGSHADAF | KSGTSASLAITGLQAEDGADYYCQSYDSSLRVV |
| DVWGQGTLVTVSS | FGGGTKVTVLG |
| QVQLQESGGGLVQPGRSLRLSCAASGFTFSSYAMHW | QSALTQPPSASGSPGQSVTISCTGTSSDVGLMHN |
| VRQAPGKGLEWVSAISSSGGSTYYADSVKGRFTISRD | VSWYQQYPGKAPKLLIYKSSSRPSGVPDRFSGSK |
| NSKNTLYLQMNSLRAEDTAVYYCAKDRRGSHADAF | SGTSASLAITGLQAEDGADYYCQSYDSSLRVVF |
| DVWGQGTLVTVSS | GGGTKVTVLG |
| QVQLQESGGGLVQPGRSLRLSCAASGFTFSSYAMHW | QSALTQPPSASGSPGQSVTISCTGTSSDVGGYNY |
| VRQAPGKGLEWVSAISSSGRSTYYADSVEGRFTISRD | VSWYQQYPGKAPKLLIYGNSNRPSGVPDRFSGS |
| NSKNTLYLQMNSLRAEDTAVYYCAKDRRGSHADAL | KSGTSASLAITGLQAEDGADYYCQSYDSSLRVV |
| NVWGQGTLVTVSS | FGGGTKVTVLG |
| EVQLVQSGAEVKKPGASVKVSCKASGYTFSDYEMH | DIVMTQTPLSLPVTPGEPASISCRSSQSLVHSNGN |
| WVRQAPGQGLEWMGAIHPGSGDTAYNQRFKGRVTI | TYLQWYLQKPGQSPQLLIYKVSNRFSGVPDRFS |
| TADKSTSTAYMELSSLRSEDTAVYYCARFYSYAYWG | GSGSGTDFTLKISRVEAEDVGVYYCSQSIYVPYT |
| QGTLVTVSA | FGQGTKLEIKR |
| QVQLQQSGTELVRPGASVKLSCKALGYTFTDYEMH | DVVMTQTPLSLPVSLGDQASISCRSSQSLVHSNG |
| WVKQTPVHGLEWIGAIHPGSGDTAYNQRFKGKATLT | NTYLQWYLQKPGQSPKLLIYKVSNRFSGVPDRF |
| ADKSSSTAYMEYSSLTSEDSAVYYCTRFYSYAYWGQ | SGRGSGTDFTLKISRVEAEDLGVYFCSQSIYVPY |
| GTLVTVSA | TFGGGTKLEIKR; |

or,
the light chain variable region and the heavy chain variable region of the amino acid sequence of the antigen recognition unit that recognizes claudin 18.2 are or have 70-100% sequence identity with a combination of the light chain variable region and the heavy chain variable region shown in any row of the following table;

| VH | VL |
|---|---|
| QVQLQQSGAELARPGASVKMSCKASGYTFTSYTMH | DIVMTQSPSSLTVTAGEKVTMSCKSSQSLLNSGNQ |
| WVKQRPGQGLEWIGYINPSSGYTNYNQKFKDKATLT | KNYLTWYQQKPGQPPKLLIYWASTRESGVPDRFT |
| ADKSSSTAYMQLSSLTSEDSAVYYCARIYYGNSFAY | GSGSGTDFTLTISSVQAEDLAVYYCQNDYSYPLTF |
| WGQGTTVTVSS | GAGTKLELKR |
| QVQLQQSGPELVKPGALVKISCKASGYTFTSYDINW | QIVLTQSPAIMSASPGEKVTMTCSASSSISYMHWY |
| VKQRPGQGLEWIGWIYPGDGSTKYNEKFKGKATLTA | QQKPGTSPKRWIYDTSKLASGVPARFSGSGSGTSY |
| DKSSSTAYMQLSSLTSENSAVYFCARGGYRYDEAMD | SLTISSMEAEDAATYYCHQRSSYPYTFGGGTKLEI |
| YWGQGTTVTVSS | KR |
| QIQLVQSGPELKKPGETVKISCKASGYTFTNYGMNW | DIVMTQSPSSLSVSAGEKVTMSCKSSQSLLNSGNQ |
| VKQAPGKGLKWMGWINTNTGEPTYAEEFKGRFAFS | KNYLAWYQQKPGQPPKLLIYGASTRESGVPDRFT |
| LETSASTAYLQINNLKNEDTATYFCARFSYGNSFAY | GSGSGTDFTLTISSVQAEDLAVYYCQNDHSYPLTF |
| WGQGTTVTVSS | GAGTKLELKR |
| DVQLQESGPDLVKPSQSLSLTCTVTGYSITSGYNWH | DIVMTQSPSSLTVTPGEKVTMTCKSSQSLFNSGNQ |
| WIRQFPGNKMEWMGYIHYTGSTNYNPSLRSRISITRD | KNYLTWYQQRPGQPPKMLIYWASTRESGVPDRFT |
| TSKNQFFLQLNSVTTDDTATYYCTRIYNGNSFPYWG | GSGSGTDFTLTISSVQAEDLAVFYCQNAYSFPYTF |
| QGTSVTVSS | GGGTKLEIKR |
| QVQLQQSGAELARPGASVKMSCKASGYTFTSYTMH | DIVMTQSPSSLTVTAGEKVTMSCKSSQSLLNSGNQ |
| WVKQRPGQGLEWIGYIDPSSGYTNYNQKFKDKATLT | KNYLTWYQQKPGQPPKLLIYWASTRESGVPDRFT |
| ADKSSSTAYMQLSSLTSEDSAVYYCARIYYGNSFAY | GSGSGTDFTLTISSVQAEDLAVYYCQNDYSYPLTF |
| WGQGTTVTVSS | GAGTKLELKR |
| QVQLQQSGAELARPGASVKMSCKASGYTFTSYTMH | DIVMTQSPSSLTVTAGEKVTMSCKSSQSLLNSGNQ |
| WVKQRPGQGLEWIGYINPASGYTNYNQKFKDKATL | KNYLTWYQQKPGQPPKLLIYWASTRESGVPDRFT |
| TADKSSSTAYMQLSSLTSEDSAVYYCARIYYGNSFA | GSGSGTDFTLTISSVQAEDLAVYYCQNDYSYPLTF |
| YWGQGTTVTVSS | GAGTKLELKR |
| QVQLQQSGAELARPGASVKMSCKASGYTFTSYTMH | DIVMTQSPSSLTVTAGEKVTMSCKSSQSLLNSGNQ |
| WVKQRPGQGLEWIGYINPASGYTNYNQKFKDKATL | KNYLTWYQQKPGQPPKLLIYWASTRESGVPDRFT |
| TADKSSSTAYMQLSSLTSEDSAVYYCARIYYGNSFA | GSGSGTDFTLTISSVQAEDLAVYYCQNDYSYPLTF |
| YWGQGTTVTVSS | GAGTKLELKR |
| QVQLVQSGAEVKKPGASVKVSCKASGYTFTSYTMH | DIVMTQSPDSLAVSLGERATINCKSSQSLLNSGNQ |
| WVRQAPGQGLEWMGYINPASGYTNYNQKFKDRVT | KNYLTWYQQKPGQPPKLLIYWASTRESGVPDRFS |
| MTRDTSTSTAYMELSSLRSEDTAVYYCARIYYGNSF | GSGSGTDFTLTISSLQAEDVAVYYCQNDYSYPLTF |
| AYWGQGTLVTVSS | GGGTKVEIKR |
| QVQLQESGPGLVKPSQTLSLTCTVSGGSISSGYNWH | DIVMTQSPDSLAVSLGERATINCKSSQSLFNSGNQ |
| WIRQPPGKGLEWIGYIHYTGSTNYNPALRSRVTISVD | KNYLTWYQQKPGQPPKLLIYWASTRESGVPDRFS |
| TSKNQFSLKLSSVTAADTAVYYCARIYNGNSFPYWG | GSGSGTDFTLTISSLQAEDVAVYYCQNAYSFPYTF |
| QGTTVTVSS | GGGTKLEIKR |
| QVQLQESGPGLIKPSQTLSLTCTVSGGSISSGYNWHW | DIVMTQSPDSLAVSLGERATINCKSSQSLFNSGNQ |
| IRQPPGKGLEWIGYIHYTGSTNYNPALRSRVTISVDTS | KNYLTWYQQKPGQPPKLLIYWASTRESGVPDRFS |
| KNQFSLKLSSVTAADTAIYYCARIYNGNSFPYWGQG | GSGSGTDFTLTISSLQAEDVAVYYCQNAYSFPYTF |
| TTVTVSS | GGGTKLEIKR. |

7. The fusion protein of claim 1, wherein the amino acid sequence of the antigen recognition unit that recognizes GPC3 is selected from or has 70-100% sequence identity with the sequence shown in the following table;
QVQLQESGGGLVQPGRSLRLSCAASGFTFSSYAMHWVRQAPGKGLEWVSA
ISGSGGSTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSTYAMTWVRQAPGKGLEWVSS
ISSSGESTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSTYAMAWVRQAPGKGLEWVSE
ISSSGSRTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSTYAMNWVRQAPGKGLEWVSA
ISMSGESTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
QVQLQESGGGLVQPGRSLRLSCAASGFTFSSYAMHWVRQAPGKGLEWVSA
ISSSGGSTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGHKFPVSWYQQYPGKAPKLLIYKNLLRPSGVPDRF
SGSKSGTSASLAITGLQAEDGADYYCQSYDSSLRVVFGGGTKVTVLG
QVQLQESGGGLVQPGRSLRLSCAASGFTFSSYAMHWVRQAPGKGLEWVSA
ISSSGGSTYYADSVKGRFTISRDNSKNTLYLQMNSLRAEDTAVYYCAKDR
RGSHADAFDVWGQGTLVTVSSGGGGSGGGGSGGGGSQSALTQPPSASGSP
GQSVTISCTGTSSDVGLMHNVSWYQQYPGKAPKLLIYKSSSRPSGVPDRF
SGSKSGTSASLAITGLQAEDGADYYCQSYDSSLRVVFGGGTKVTVLG
QVQLQESGGGLVQPGRSLRLSCAASGFTFSSYAMHWVRQAPGKGLEWVSA
ISSSGRSTYYADSVEGRFTISRDNSKNTLYLQMNSLRAEDTAVYYCAKDR
RGSHADALNVWGQGTLVTVSSGGGGSGGGGSGGGGSQSALTQPPSASGSP
GQSVTISCTGTSSDVGGYNYVSWYQQYPGKAPKLLIYGNSNRPSGVPDRF
SGSKSGTSASLAITGLQAEDGADYYCQSYDSSLRVVFGGGTKVTVLG
EVQLVQSGAEVKKPGASVKVSCKASGYTFSDYEMHWVRQAPGQGLEWMGA
IHPGSGDTAYNQRFKGRVTITADKSTSTAYMELSSLRSEDTAVYYCARFY
SYAYWGQGTLVTVSAGGGGSGGGGSGGGGSDIVMTQTPLSLPVTPGEPAS
ISCRSSQSLVHSNGNTYLQWYLQKPGQSPQLLIYKVSNRFSGVPDRFSGS
GSGTDFTLKISRVEAEDVGVYYCSQSIYVPYTFGQGTKLEIKR
QVQLQQSGTELVRPGASVKLSCKALGYTFTDYEMHWVKQTPVHGLEWIGA
IHPGSGDTAYNQRFKGKATLTADKSSSTAYMEYSSLTSEDSAVYYCTRFY
SYAYWGQGTLVTVSAGGGGSGGGGSGGGGSDVVMTQTPLSLPVSLGDQAS
ISCRSSQSLVHSNGNTYLQWYLQKPGQSPKLLIYKVSNRFSGVPDRFSGR
GSGTDFTLKISRVEAEDLGVYFCSQSIYVPYTFGGGTKLEIKR
or,
the amino acid sequence of the antigen recognition unit that recognizes claudin18.2 is selected from or has 70-100% sequence identity with the sequence shown in the following table;
QVQLQQSGAELARPGASVKMSCKASGYTFTSYTMHWVKQRPGQGLEWIGY
INPSSGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLQQSGPELVKPGALVKISCKASGYTFTSYDINWVKQRPGQGLEWIGW
IYPGDGSTKYNEKFKGKATLTADKSSSTAYMQLSSLTSENSAVYFCARGG
YRYDEAMDYWGQGTTVTVSSGGGGSGGGGSGGGGSQIVLTQSPAIMSASP
GEKVTMTCSASSSISYMHWYQQKPGTSPKRWIYDTSKLASGVPARFSGSG
SGTSYSLTISSMEAEDAATYYCHQRSSYPYTFGGGTKLEIKR
QIQLVQSGPELKKPGETVKISCKASGYTFTNYGMNWVKQAPGKGLKWMGW
INTNTGEPTYAEEFKGRFAFSLETSASTAYLQINNLKNEDTATYFCARFS
YGNSFAYWGQGTTVTVSSGGGGSGGGGSGGGGSDIVMTQSPSSLSVSAGE
KVTMSCKSSQSLLNSGNQKNYLAWYQQKPGQPPKLLIYGASTRESGVPDR
FTGSGSGTDFTLTISSVQAEDLAVYYCQNDHSYPLTFGAGTKLELKR
DVQLQESGPDLVKPSQSLSLTCTVTGYSITSGYNWHWIRQFPGNKMEWMG
YIHYTGSTNYNPSLRSRISITRDTSKNQFFLQLNSVTTDDTATYYCTRIY
NGNSFPYWGQGTSVTVSSGGGGSGGGGSGGGGSDIVMTQSPSSLTVTPGE
KVTMTCKSSQSLFNSGNQKNYLTWYQQRPGQPPKMLIYWASTRESGVPDR
FTGSGSGTDFTLTISSVQAEDLAVFYCQNAYSFPYTFGGGTKLEIKR
QVQLQQSGAELARPGASVKMSCKASGYTFTSYTMHWVKQRPGQGLEWIGY
IDPSSGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLQQSGAELARPGASVKMSCKASGYTFTSYTMHWVKQRPGQGLEWIGY
INPASGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLQQSGAELARPGASVKMSCKASGYTFTSYTMHWVKQRPGQGLEWIGY
INPASGYTNYNQKFKDKATLTADKSSSTAYMQLSSLTSEDSAVYYCARIY
YGNSFAYWGQGTTVTVSSGGGGSGGGGSGGGGSDIVMTQSPSSLTVTAGE
KVTMSCKSSQSLLNSGNQKNYLTWYQQKPGQPPKLLIYWASTRESGVPDR
FTGSGSGTDFTLTISSVQAEDLAVYYCQNDYSYPLTFGAGTKLELKR
QVQLVQSGAEVKKPGASVKVSCKASGYTFTSYTMHWVRQAPGQGLEWMGY
INPASGYTNYNQKFKDRVTMTRDTSTSTAYMELSSLRSEDTAVYYCARIY
YGNSFAYWGQGTLVTVSSGGGGSGGGGSGGGGSDIVMTQSPDSLAVSLGE
RATINCKSSQSLLNSGNQKNYLTWYQQKPGQPPKLLIYWASTRESGVPDR
FSGSGSGTDFTLTISSLQAEDVAVYYCQNDYSYPLTFGGGTKVEIKR
QVQLQESGPGLVKPSQTLSLTCTVSGGSISSGYNWHWIRQPPGKGLEWIG
YIHYTGSTNYNPALRSRVTISVDTSKNQFSLKLSSVTAADTAVYYCARIY
NGNSFPYWGQGTTVTVSSGGGGSGGGGSGGGGSDIVMTQSPDSLAVSLGE
RATINCKSSQSLFNSGNQKNYLTWYQQKPGQPPKLLIYWASTRESGVPDR
FSGSGSGTDFTLTISSLQAEDVAVYYCQNAYSFPYTFGGGTKLEIKR
QVQLQESGPGLIKPSQTLSLTCTVSGGSISSGYNWHWIRQPPGKGLEWIG
YIHYTGSTNYNPALRSRVTISVDTSKNQFSLKLSSVTAADTAIYYCARIY
NGNSFPYWGQGTTVTVSSGGGGSGGGGSGGGGSDIVMTQSPDSLAVSLGE
RATINCKSSQSLFNSGNQKNYLTWYQQKPGQPPKLLIYWASTRESGVPDR
FSGSGSGTDFTLTISSLQAEDVAVYYCQNAYSFPYTFGGGTKLEIKR.

8. A combination of the fusion protein of any one of claims 1-7 and other factors, wherein the other factors includes cytokines, transcription factors, chemokines, and/or combinations thereof, and the expression cassette is constitutively or inducibly expressed; preferably, the promoter of the expression cassette is an immune cell inducible promoter; preferably, the immune cell inducible promoter is NFAT6 promoter; and preferably, the NFAT6 promoter is reversely regulated.

9. A nucleic acid molecule expressing the fusion protein of any one of claims 1-8 or the combination of claim 2.

10. A vector comprising the nucleic acid molecule of claim 9.

11. A cell comprising the vector of claim 10 or having the nucleic acid molecule of claim 9 integrated into its genome.

12. A protein complex comprising:
i) the fusion protein TFP molecule of any one of claims 1-7; and
ii) at least one endogenous TCR subunit or endogenous TCR complex.

13. A method for preparing cells, comprising transducing T cells with the vector of claim 10 or the nucleic acid molecule of claim 9.

14. A method for producing an RNA-engineered cell population, comprising introducing in vitro transcribed RNA or synthetic RNA into the cell,
(i) wherein the RNA includes the nucleic acid of claim 9.

15. A method for providing anti-tumor immunities in a mammal, comprising administering to the mammal an effective amount of the fusion protein of any one of claims 1-7 and the combination of claim 8, the nucleic acid molecule of claim 9, the vector of claim 10, or the cell of claim 11.

16. A method for treating a mammal suffering from a disease related to the expression of GPC3 or claudin 18.2, comprising administering to the mammal an effective amount of the fusion protein of any one of claims 1-7, the combination of claim 8, the nucleic acid molecule of claim 9, the vector of claim 10, or the cell of claim 11.

17. The fusion protein of any one of claims 1-7, the combination of claim 8, the nucleic acid molecule of claim 9, the vector of claim 10, or the cell of claim 11 as a medicament.
